(19) United States
(12) Patent Application Publication
(10) Pub. No.: US 2026/0232847 A1
(43) Pub. Date: Aug. 13, 2026

(54) Nanoparticles For Imaging And Treatment Of Arthritis And Inflammatory Joint Diseases

(71) Applicant: Cornell University, Ithaca, NY (US)

(72) Inventors: Ulrich B. Wiesner, Ithaca, NY (US); Lawrence Bonassar, Ithaca, NY (US); Aiyana Fortin, Ithaca, NY (US)

(21) Appl. No.: 19/154,961

(22) PCT Filed: Feb. 9, 2024
(86) PCT No.: PCT/US2024/015240

§ 371 Date: Aug. 8, 2025

Related U.S. Application Data
(60) Provisional application No. 63/444,372, filed on Feb. 9, 2023.
(60) Provisional application No. 63/550,455, filed on Feb. 6, 2024.

(51) Int. Cl. A61K 49/00; A61B 5/00; A61K 9/51; A61K 47/69

(52) U.S. Cl. CPC A61K 49/0093; A61B 5/0071; A61K 9/5115; A61K 9/5146; A61K 47/6925

(57) ABSTRACT

Therapeutic methods, imaging methods, and inorganic nanotherapeutic imaging agents and inorganic nanoimaging agents. Inorganic nanotherapeutic imaging agents and inorganic nanoimaging agents are used in therapeutic methods and imaging methods. The inorganic nanotherapeutic imaging agents and inorganic nanoimaging agents are, for example, silica or aluminosilicate nanoparticles functionalized with fluorescent group(s), targeting groups, and therapeutic groups. Therapeutic methods can be used to treat and imaging methods can be used to investigate or diagnose inflammatory joint diseases, such as, for example, rheumatoid arthritis, osteoarthritis, or both, in a subject.

### CROSS REFERENCE TO RELATED APPLICATIONS

This application claims the benefit of U.S. Provisional Application No. 63/444,372, filed Feb. 9, 2023 and U.S. Provisional Application No. 63/550,455, filed Feb. 6, 2024; the contents of the above-identified applications are hereby fully incorporated herein by reference in their entirety.

### SEQUENCE LISTING

The instant application contains a Sequence Listing, which is submitted in .xml format and is hereby incorporated by reference in its entirety. Said .xml file is named “018617_01747_ST26.xml”, was created on Feb. 9, 2024, and is 40,754 bytes in size.

### BACKGROUND OF THE DISCLOSURE

Development of non-surgical interventions for treatment of osteoarthritis (OA) remains a persistent challenge despite decades of efforts. There are no clinically available disease-modifying OA drugs (DMOADs) that prevent cartilage loss—the hallmark of this irreversible disease. Pain-relieving treatments such as corticosteroid injections offer short term mobility-enhancing effects but may have injurious side effects. Alternatively, intraarticular hyaluronic acid (HA) viscosupplementation is clinically available, but the efficacy and mechanism of action of HAs are debated. Even disease-modifying anti-rheumatic drugs (DMARDs) that exhibit therapeutic amelioration of rheumatoid arthritis (RA) of the knee, such as anakinra (Kineret) and adalimumab (Humira) have modest therapeutic effect as treatments for knee OA. Unfortunately, many promising OA therapeutic candidates have shown efficacy in small animal models (e.g., mice, rats, and rabbits) that failed to translate to larger animal models and humans in pre-clinical and clinical trials. Challenges in translation arise from two key reasons: 1) the therapeutic target in RA is the highly vascularized and accessible synovium and 2) cartilage thickness varies by an order of magnitude from rodents to large animals and humans. Thus, a platform that overcomes both targeting and transport challenges to achieve efficacious drug delivery is needed in knee OA.

OA is unique from other forms of arthritis due to its chondrocyte-mediated pathogenesis. The initiation of inflammatory processes, either following traumatic joint injury or due to aging-related joint changes, creates a positive feedback loop wherein chondrocytes perpetuate inflammatory signaling and secrete enzymes that degrade the surrounding cartilage matrix. Over decades, inflammation and enzymatic degradation lead to bone-on-bone articulation, loss of mobility, and the severe joint pain commonly associated with late-stage OA. Thus, injected therapeutics must reach chondrocytes and disrupt this inflammatory feedback loop to be effective in treating OA. However, cartilage has low permeability due to tissue avascularity and dense, negatively charged extracellular matrix (ECM), which is size- and charge-restrictive to solute penetration. While many therapeutics (e.g., growth factors, receptor antagonists, and antibodies) would penetrate the articular surface of cartilage given ample diffusion time, tissue exposure time is limited by the rapid clearance rate of synovial fluid. Given the chondrocyte-mediated pathogenesis of osteoarthritis, poor penetration of injected solutes in cartilage directly inhibits therapeutic potential because injected solutes do not reach chondrocytes before being cleared from the joint.

Synovial fluid is a viscous, protein rich solution that lubricates the articulating surfaces of joint tissues and provides convective transport of nutrients, cytokines, and metabolic wastes. Small molecules (i.e, under 10 kDa) are cleared via synovial vasculature while large molecules (i.e., over 40 kDa) are cleared via synovial lymphatics. These clearance mechanisms result in joint residence times on the order of hours for injected solutes, with modest increase in joint half-life (10-fold) observed over 5 orders of magnitude increase in hydrodynamic radius. Synovial fluid clearance rates are conserved across species and joint size, yielding similar synovial clearance times of injected solutes. In contrast, cartilage thickness increases with joint volume. For example, the thickness of mouse and rat cartilage is 50-100 μm, rabbit cartilage is 350-700 μm, and human cartilage thickness is 1.5-2 mm. This 10-fold difference in thickness could account for a 100-fold difference in tissue penetration of injected solutes due to diffusion scaling with the square of distance. While therapeutics with modest cartilage diffusivity may be effective in small animals with thin cartilage, high cartilage diffusivity is required to travel 10× the distance in the same time, i.e., for translation to large animals and humans. Since this high diffusivity in cartilage is often not inherent to the therapeutic solute, engineered drug delivery vehicles offer tunable transport kinetics. For example, engineered positively charged peptide sequences (CPC+14) enhanced cartilage partitioning by mimicking Avidin's native properties. Engineered vehicles that effectively deliver potent DMARDs to chondrocytes would change the landscape of drug delivery in the knee.

Several groups have functionalized delivery vehicles further to interact with the cartilage ECM or chondrocytes, either by charge or reversible binding interactions. Several groups have observed higher tissue localization mediated by reversible interactions of collagen-II-binding or chondrocyte-targeting peptides. Nano- and microparticle systems have increased joint half-lives of conjugated therapeutics from hours to days, thus extending joint retention by 2-4 weeks. However, this broad approach is insufficient to achieve clinically feasible joint retention times (e.g., 3-6 months) or therapeutic results in large animals and humans. Engineered particles with targeting and therapeutic functionalities must also exhibit high cartilage diffusivity to leverage the principles of depot formation and further extend total joint retention time. The most promising nanomaterial delivery vehicles thus far have shown high cartilage penetration. For example, the half-life of an established DMARD, anakinra (Kineret), in the knee is 1 day. When conjugated to block co-polymer nanoparticles (dₕ~270 nm), joint half-life of anakinra was extended to 3 days. In another system, conjugation of anakinra to cationic peptide carrier (CPC)+14, extended anti-inflammatory effects from 2 days to 16 days in vitro. Thus, vehicles that increase joint half-life and tissue access have the potential to translate the therapeutic potency of receptor antagonists and cytokine blockers to applications in OA.

To support translation to knee OA, it is necessary to consider solute distribution, transport, and clearance in intact joints in vivo. Many studies have found that arthritic pathology can affect clearance of injected solutes due to tissue degradation, intraarticular pressure, increased vascularization and lymphatic vessel function, and synovial thickening. Surgical models of arthritis are employed to quickly induce arthritis-like pathology in small animals. The rat anterior cruciate ligament transection model (ACLT) causes mild OA-pathology on the order of 1-3 months and can be performed in both ipsilateral and contralateral knees. Many groups have utilized this rodent model in conjunction with longitudinal fluorescence imaging to characterize whole joint clearance pharmacokinetics of injected solutes.

The field of injectable OA therapeutics has an ongoing and unmet need for an engineered drug delivery vehicle with high cartilage diffusivity and extended residence time, creating high potential for translation to large animal models and humans.

### SUMMARY OF THE DISCLOSURE

The present disclosure provides, inter alia, treatment methods and imaging methods. Also provided are inorganic nanotherapeutic agents and inorganic nanoimaging agents and uses thereof.

In an aspect, the present disclosure provides methods. In various examples, a method is a treatment method. In various examples, a method is an imaging method. In various examples, a method comprises both treatment and imaging methods. In various examples, a method is a theranostic method or the like.

In various examples, a method uses inorganic nanotherapeutic agents and/or inorganic nanoimaging agents. In various examples, a method comprises administration of inorganic nanotherapeutic agent(s) and/or inorganic nanoimaging agent(s) to a subject.

In various examples, a therapeutic method is used to treat an inflammatory joint disease (such as, for example, arthritis (e.g., rheumatoid arthritis and osteoarthritis, and the like, and both) and the like), a cancer (which may be related to a joint, musculoskeletal tissue, or the like), and the like, and any conditions, disorders, or side effects, or the like related to same, or the like. In various examples, an inorganic nanotherapeutic agent used in a therapeutic method comprises targeting group(s) and therapeutic group(s).

In various example, an imaging method is used to image one or more joints(s) (or any portion thereof) (such as, for example, one or more cell(s) associated with the bone(s) (e.g., embedded cell(s) or the like) or the like), one or more tissue(s) (or any portion thereof) associated with the joint(s), or any combination thereof, for example, in a sample or a portion thereof or an individual or a portion thereof. In various examples, an inorganic nanoimaging agent used in an imaging method comprises fluorescent group(s). In various examples, the targeting group(s) and therapeutic group(s). In various examples, the imaging is fluorescence imaging (such as, for example, optical super-resolution imaging).

In an aspect, the present disclosure provides compositions. In various examples, a composition comprises inorganic nanotherapeutic agent(s) and/or inorganic nanoimaging agent(s). In various examples, a composition is a pharmaceutical composition.

In an aspect, the present disclosure provides kits. In various examples, a kit comprises inorganic nanotherapeutic agent(s) and/or inorganic nanoimaging agent(s) and/or composition(s) and, optionally, instructions for use of the inorganic nanotherapeutic agent(s) and/or the inorganic nanoimaging agent(s) and/or the composition(s) for carrying out a method of the present disclosure.

### BRIEF DESCRIPTION OF THE FIGURES

For a fuller understanding of the nature and objects of the disclosure, reference should be made to the following detailed description taken in conjunction with the accompanying figures.

FIG. 1A shows a molecular rendering of PEGylated C′ Dot: fluorescent poly(ethylene) glycol coated core-shell silica nanoparticle (dh~6 nm (nm=nanometer(s))). FIG. 1A(i) shows 1-2 Cyanine 5 fluorophores covalently encapsulated by silica core-shell structure, which was coated with a dense layer of covalently attached PEG (6-9 glycol repeats) chains, as shown in FIG. 1A(ii).

FIG. 1B shows a schematic of patellofemoral groove cartilage dissected from neonatal bovine knee joints for use in (i) diffusion experiments and (ii) chondrocyte uptake and retention experiments.

FIG. 1C shows a schematic of healthy and surgically injured (sham or anterior cruciate ligament transection, ACL-T) rat knees receiving a single injection of C′ Dots. Injected knees were imaged longitudinally and analyzed to characterize joint pharmacokinetics of C′ Dots in vivo.

FIG. 2A shows a schematic of patellofemoral groove cartilage explant dissection, preparation, and imaging for diffusion studies. Confocal fluorescence was recorded in the center of the bisected cartilage explant.

FIG. 2B shows representative merged reflective and fluorescence confocal micrographs of healthy cartilage explants after incubation with C′ Dots for 0.5 h (h=hour(s)), 1 h, or 2 h illustrating diffusion of C′ Dots into the explant from the articular surface.

FIG. 2C shows normalized C′ Dot fluorescence profiles (perpendicular to the articular surface) from representative confocal micrographs shown in FIG. 2B.

FIG. 2D shows transport analysis (Fickian 1D) of a single fluorescence profile from tissue depth of 100-400 μm (μm=micrometer(s)).

FIG. 2E shows the calculated mean diffusivities at incubation times from 0.5 to 2 hours, shared letters denote no significant difference between groups (α=0.05).

FIG. 3 shows a schematic of image analysis, wherein C′ Dot fluorescence was isolated and segmented into 6 μm rectangles parallel to the articular surface. The average pixel intensity for each segment was used to construct a fluorescence profile (y) through the depth (x) of each sample.

FIG. 4A (left) shows merged confocal fluorescence micrographs showing internalized C′ Dots (z-stack spans 10 μm; green—Calcein, AM, red—Cyanine 5). Orthogonal views highlight intracellular localization of C′ Dots. Arrows indicate C′ Dot fluorescence. FIG. 4A (right) shows C′ Dots retained intracellularly for 5 days after removal from the media.

FIG. 4B shows merged confocal micrographs showing internalized C′ Dots at 0 d (i.e. 24 hours loading) and 5 d with co-staining of (left) endosomes and (right) lysosomes. Arrows indicate colocalized C′ Dots & stain. Notably, C′ Dots are present in endosomes, lysosomes, and unlabeled vesicular structures.

FIG. 4C (i) shows representative C′ Dot signal in cells and identified subcellular vesicles. Notably, C′ Dots are highly heterogeneous within cells. FIG. 4C (ii) shows integrated pixel intensity of C′ Dot fluorescence within ECM and live cells (mean number of cells per explant: 0 d=750 cells. 1 d=1100 cells, and 5 d=770 cells). FIG. 4C (iii) shows area-normalized C′ Dot intensity of within ECM and cells trended towards decreasing. FIG. 4C (iv) shows integrated pixel intensity of C′ Dot fluorescence within vesicular structures (d=1.26 μm) in live cells (left axis) and number of vesicles per cell (right axis). Shared letters denote no significant difference between groups (α=0.05).

FIG. 5A shows representative IVIS fluorescence overlay from 0-8 weeks post-injection in unilaterally injected healthy rat. Fluorescence signal (epi-fluorescence) in the left knee was still notable at 8 weeks.

FIG. 5B shows dissected healthy knees imaged 14 weeks post-injection. C′ Dot fluorescence was concentrated in PL, ACL, and PCL. S, and M, and more diffusely present in the MFC, LFC, and TP. C) Integrated intra-tissue C′ Dot signal from B), illustrating ~20% localized to menisci, ~17% in combined articular cartilage (MFC+LFC+TP) and ~47% in synovium after 14 weeks. Abbreviations: PL: patellar ligament, ACL: anterior cruciate ligament. PCL: posterior cruciate ligament, S: synovium, M: meniscus. TP: tibial plateau, MFC: medial femoral condyle, LFC: lateral femoral condyle.

FIG. 5C shows integrated intra-tissue C′ Dot signal from FIG. 4B, illustrating ~20% localized to menisci, ~17% in combined articular cartilage (MFC+LFC+TP) and ~47% in synovium after 14 weeks.

FIG. 6A shows normalized background subtracted fluorescence quantification from manually aligned ellipsoidal ROIs in IVIS fluorescence images of representative rats that received intraarticular injections of C′ Dots or Cy5 dye (Groups: Cy5: Naïve, C′ Dots: Naïve, ACLT, Sham). One-component (Cy5) and two-component (C′ Dots) exponential decay model fits are shown in black.

FIG. 6B shows normalized fit coefficients A₁, A₂, and c for model fits illustrated in FIG. 5A for all animals.

FIG. 6C shows time constants τ₁ and τ₂ for model fits illustrated in FIG. 6A for all animals. Observed τ of Cy5 dye was lower than τ₁ for C′ Dots (p<0.001) but no differences were observed in C′ Dot τ₁ or τ₂ for unoperated or operated conditions.

FIGS. 7A and 7B show the fluorescence correlation spectroscopy and gel permeation chromatography curves for representative C′ Dot batches.

FIGS. 8A and 8B show explant preparation for diffusion studies, confocal imaging workflow, and graphic pseudocode for fluorescence profile analysis in custom MATLAB code.

FIGS. 9A and 9B show animal groups for pharmacokinetic study, experimental timeline, and imaging timepoints.

FIGS. 10A to 10D show IVIS Fluorescence imaging of all dissected joints (n=4) at 14 weeks post-injection and estimated signal quantification by tissue type.

FIGS. 11A and 11B show representative histological sections from a rat that received bilateral C′ Dot injection for joint pharmacokinetic study one week following ACL-transection surgery (FIG. 10B) and sham surgery in the contralateral joint (FIG. 10A). This surgery causes destabilization of the joint which ultimately leads to cartilage degradation. Cartilage degradation was observed in the medial tibial compartment, highlighted in the expanded histological images (lower figures).

FIG. 12A shows a representative confocal merged reflectance and fluorescence images of healthy cartilage after incubation with C′ dots from 0-2 hours.

FIG. 12B shows representative normalized average fluorescence from FIG. 12A. Arrows highlight C′ Dot loss from the surface zone at short incubation times.

FIG. 12C shows representative 1D Fickian diffusion fit.

FIG. 12D shows mean effective diffusivities for each incubation time. Shared letters denote no statistical difference between groups (α=0.05).

FIG. 12E shows a calculated average effective diffusivity from 0-2 hours. n=3-6, shared letters denote no significant difference (α=0.01).

FIG. 13A shows representative confocal merged reflectance and fluorescence images of healthy cartilage after 2 h incubation with 0 peptide C′ dots, 2 Col-II C′ dots, 3 Col-II C′ dots, and 14 Col-II C′ dots.

FIG. 13B shows representative normalized average fluorescence from FIG. 13A.

FIG. 13C shows representative 1D Fickian diffusion fit for 14 peptide C′ dots, fit from 100-400 μm; this region exhibits minimal variation in effective diffusivity with depth.

FIG. 13D shows mean effective diffusivities for each C′ Dot formulation. Shared letters denote no statistical difference between groups (α=0.05).

FIG. 13E shows calculated average effective diffusivity of C′ dots, 2 Col-II C′ dots, 3 Col-II C′ dots, and 14 Col-II C′ dots. n=3-6, shared letters denote no significant difference (α=0.01).

### DETAILED DESCRIPTION OF THE DISCLOSURE

Although claimed subject matter will be described in terms of certain examples, other examples, including examples that do not provide all of the benefits and features set forth herein, are also within the scope of this disclosure. Various structural, logical, and process step changes may be made without departing from the scope of the disclosure.

As used herein, unless otherwise stated, “about,” “approximately,” “substantially.” or the like, when used in connection with a measurable variable such as, for example, a parameter, an amount, a temporal duration, or the like, are meant to encompass variations of, for example, a specified value including, for example, those within experimental error (which can be determined by for example, a given data set, an art accepted standard, and/or with a given confidence interval (e.g. 90%, 95%, or more confidence interval from the mean), such as, for example, variations of +/−10% or less, +1-5% or less, +/−1% or less, and +/−0.1% or less of and from the specified value) or to encompass alternatives to the members of the list that would be recognized by one of ordinary skill in the art as alternatives, where the members and the alternatives may define a genus or sub-genus, insofar such variations are appropriate to perform in the context of the disclosure. As used herein, unless otherwise stated, the terms “about,” “approximate,” “at or about,” and “substantially” can mean that the amount or value in question can be the exact value or a value that provides equivalent results or effects as recited in the sample claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error, and the like, and other factors known to those of skill in the art such that, for example, equivalent results, effects, or the like are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value) of a range. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also, unless otherwise stated, include individual values (e.g., about 1%, about 2%, about 3%, and about 4%) and the sub-ranges (e.g., about 0.5% to about 1.1%; about 0.5% to about 2.4%; about 0.5% to about 3.2%, and about 0.5% to about 4.4%, and other possible sub-ranges) within the indicated range. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about, it will be understood that the particular value forms a further disclosure. For example, if the value “about 10” is disclosed, then “10” is also disclosed.

As used herein, unless otherwise stated, the term “group” or “moiety” refers to a chemical entity that is monovalent (i.e., has one terminus that can be covalently bonded to other chemical species), divalent, or polyvalent (i.e., has two or more termini that can be covalently bonded to other chemical species). The term “group” also includes radicals (e.g., monovalent and multivalent, such as, for example, divalent radicals, trivalent radicals, and the like). Illustrative examples of groups or moieties include:

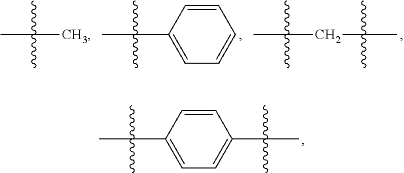

and the like.

The present disclosure provides, inter alia, imaging and/or treatment methods. The present disclosure also provides inorganic nanoimaging agents and compositions thereof and uses thereof.

In an aspect, the present disclosure provides methods. In various examples, a method is a treatment method. In various examples, a method is an imaging method. In various examples, a method comprises both treatment and imaging methods. In various examples, a method is a theranostic method or the like. Non-limiting examples of methods are described herein.

In various examples, a method is a treatment method. In various examples, inorganic nanotherapeutic agents are used to treat a current or potential disease, disease state, condition, disorder, side effect, or any combination thereof, in a subject. In various examples, a treatment method is a theranostic method or the like. Non-limiting examples of current or potential diseases, disease states, conditions, disorders, and side effects include inflammatory joint diseases (such as, for example, arthritis (e.g., rheumatoid arthritis and osteoarthritis, and the like, and both) and the like), cancers (which may be related to a joint, musculoskeletal tissue, or the like), and the like, and any conditions, disorders, or side effects, or the like related to same, or the like.

In various examples, a method of treating a subject diagnosed with or suspected of having or developing inflammatory joint diseases (such as, for example, arthritis (e.g., rheumatoid arthritis and osteoarthritis, and the like, and both) and the like) comprises: administering one or more inorganic nanotherapeutic agent(s) (and optionally, one or more inorganic nanoimaging agent(s)) to the subject, where one or more symptom(s) of the individual's inflammatory joint disease (such as, for example, arthritis (e.g., rheumatoid arthritis and osteoarthritis, and the like, and both) and the like) is treated. In various examples, a treatment method further comprises an imaging method and/or diagnostic method, which may be an imaging method and/or diagnostic method of the present disclosure. In various examples, a treatment method further comprises an intravital imaging method. Non-limiting examples of imaging methods and diagnostic methods are provided herein.

A treatment method can used to treat various current or potential disease, disease state, condition, disorder, side effect, or any combination thereof, in a subject. In various examples, a method can be used to treat an inflammatory joint disease (such as, for example, arthritis (e.g., rheumatoid arthritis and osteoarthritis, and the like, or both) and the like), cancer (such as, for example, a cancer associated with a joint, a musculoskeletal tissue, or the like) or the like.

In various examples, a subject is treated prophylactically. In various examples a subject has suffered an injury (e.g., the injured subject is predisposed to developing post-traumatic osteoarthritis (e.g., a subtype of osteoarthritis) (e.g., joint deterioration consistent with osteoarthritis), but may or may not have diagnosable tissue changes until years after the injury). In various examples, a subject (who may not have diagnosable tissue changes) is suspected of developing rheumatoid arthritis and/or osteoarthritis (such as, for example, post-traumatic osteoarthritis or the like).

A subject (e.g., a subject in need of treatment or the like) (also referred to, in the alternative, as an individual), may be a human or other animal (which may be a non-human mammal). In various examples, a subject is in need of treatment for (e.g., diagnosed with or suspected of having) a current or potential disease, disease state, condition, disorder, side effect, or any combination thereof). In various examples, a subject is in need of treatment for (e.g., diagnosed with or suspected of having) an inflammatory joint disease (such as, for example, arthritis (e.g., rheumatoid arthritis and osteoarthritis, and the like, or both) and the like), cancer (such as, for example, a cancer associated with a joint, a musculoskeletal tissue, or the like) or the like. Non-limiting examples of non-human animals (which may be mammals) include cows, pigs, mice, rats, rabbits, cats, dogs, and other agricultural animals, pets (such as, for example, dogs, cats, and the like), service animals, and the like.

“Treating” or “treatment” of a current or potential disease, a disease state, a condition, a disorder, a side effect, or any combination thereof (such as, for example, an inflammatory joint disease (such as, for example, arthritis (e.g., rheumatoid arthritis and osteoarthritis, and the like, and both) and the like), cancer (such as, for example, a cancer associated with a joint, a musculoskeletal tissue, or the like) or the like) in a subject, in various non-limiting examples, to at least partially, substantially, or completely ameliorating (e.g., arresting, reversing, alleviating, or the like) the current or potential disease, a disease state, a condition, a disorder, a side effect, or any combination thereof (such as, for example, an inflammatory joint disease (such as, for example, arthritis (e.g., rheumatoid arthritis and osteoarthritis, and the like or both) and the like), cancer (such as, for example, a cancer associated with a joint, a musculoskeletal tissue, or the like) or the like) or reducing the manifestation, extent or severity of one or more clinical symptom(s) of the current or potential disease, a disease state, a condition, a disorder, a side effect, or any combination thereof (such as, for example, an inflammatory joint disease (such as, for example, arthritis (e.g., rheumatoid arthritis and osteoarthritis, and the like, or both) and the like), cancer (such as, for example, a cancer associated with a joint, a musculoskeletal tissue, or the like) or the like).

In various other examples, “treating” or “treatment” refers to ameliorating one or more physical parameter(s), which, independently, may or may not be discernible by the subject. In yet other examples, “treating” or “treatment” refers to modulating the current or potential disease, a disease state, a condition, a disorder, a side effect, or any combination thereof (such as, for example, an inflammatory joint disease (such as, for example, arthritis (e.g., rheumatoid arthritis and osteoarthritis, and the like, or both) and the like), cancer (such as, for example, a cancer associated with a joint, a musculoskeletal tissue, or the like) or the like), either physically, (e.g., stabilization of one or more discemible symptom(s), or the like), physiologically, (e.g., stabilization of one or more physical parameter, or the like), or both. In yet other examples, treating” or “treatment” relates to slowing the progression of the current or potential disease, a disease state, a condition, a disorder, a side effect, or any combination thereof (such as, for example, an inflammatory joint disease (such as, for example, arthritis (e.g., rheumatoid arthritis and osteoarthritis, and the like, or both), cancer (such as, for example, a cancer associated with a joint, a musculoskeletal tissue, or the like) or the like). In various examples, treating comprises administration of an effective amount of an inorganic nanotherapeutic agent or agents or composition(s) comprising an inorganic nanotherapeutic agent or agents.

As used herein, unless otherwise indicated, the term “effective amount” means that amount of inorganic nanotherapeutic agent(s) and/or composition(s) that will elicit the biological or medical response of a subject (or a tissue, system, or the like, thereof) that is being sought, for instance, by a researcher, clinician, or the like. An effective amount may be a therapeutically effective amount. The term “therapeutically effective amount” includes any amount which, as compared to a corresponding subject who has not received such amount, results in improved treatment, healing, prevention, or amelioration of the current or potential disease, a disease state, a condition, a disorder, a side effect, or any combination thereof (such as, for example, an inflammatory joint disease (such as, for example, arthritis (e.g., rheumatoid arthritis and osteoarthritis, and the like, or both), cancer (such as, for example, a cancer associated with a joint, a musculoskeletal tissue, or the like) or the like) or a decrease in the rate of advancement of the current or potential disease, a disease state, a condition, a disorder, a side effect, or any combination thereof (such as, for example, an inflammatory joint disease (such as, for example, arthritis (e.g., rheumatoid arthritis and osteoarthritis, and the like, or both), cancer (such as, for example, a cancer associated with a joint, a musculoskeletal tissue, or the like) or the like). The term also includes within its scope amounts effective to enhance normal physiological function.

A physician or veterinarian having ordinary skill in the art can readily determine and prescribe the effective amount of the compound(s) and/or composition(s) required. The selected dosage level can depend upon a variety of factors including, but not limited to, the activity of the particular composition employed, the time of administration, the rate of excretion or metabolism of the particular composition being employed, the rate and extent of absorption, the duration of the treatment, other drugs, compounds and/or materials used in combination with the particular composition employed, the age, sex, weight, condition, general health and prior medical history of the patient being treated, and like factors well known in the medical arts. For example, the physician or veterinarian could start doses of the composition employed at levels lower than that required in order to achieve the desired therapeutic effect and gradually increase the dosage until the desired effect is achieved.

In the case where the current or potential disease is cancer, in various examples, the method further comprises one or more chemotherapy treatment(s), one or more radiation treatment(s), one or more photodynamic therapy treatment(s), one or more surgical intervention(s), or the like, or any combination thereof. These individual treatment(s) (e.g., the one or more additional step(s)) may be carried out before and/or after and/or during obtaining the image(s).

In various examples, a method is an imaging method. In various examples, inorganic nanoimaging agents are used to image a subject. In various examples, an imaging method is a theranostic method or the like. In various examples, an imaging method is a fluorescence imaging method. In various examples, an imaging method is an intravital fluorescence imaging method.

In various example, a method is a method of imaging of one or more joints(s) (or any portion thereof) (such as, for example, one or more cell(s) associated with the bone(s) (e.g., embedded cell(s) or the like) or the like), one or more tissue(s) (or any portion thereof) associated with the joint(s), or any combination thereof, for example, in a sample or a portion thereof or an individual or a portion thereof, using one or more inorganic nanoimaging agent(s). In various examples, a method for imaging one or more joints(s) or any portion thereof, one or more musculoskeletal tissue(s) or any portion thereof, or any combination thereof in a sample or a subject comprises: contacting the sample or the subject with one or more inorganic nanoimaging agent(s) (and optionally, one or more inorganic nanotherapeutic agent(s)); and imaging the sample or the subject. In various examples a method further comprises targeting, diagnosing, treating, preventing, or any combination thereof, a current or potential disease, disease state, condition, disorder, side effect, or any combination thereof, in the individual. Non-limiting examples of treatment and diagnostic methods are provided herein.

In various examples, a sample is obtained from an individual. In various examples, the sample is a biopsy obtained from an individual.

In various examples, an imaging method images one or more joints(s) (or any portion thereof) (such as, for example, one or more cell(s) associated with the bone(s) (e.g., embedded cell(s) or the like) or the like), one or more tissue(s) (or any portion thereof) associated with the joint(s) (such as, for example, musculoskeletal tissue(s) or the like), or any combination thereof. Non-limiting examples of musculoskeletal tissue(s) include cartilage, synovial membrane, ligaments, tendons, tendon sheaths, bursas, bone, synovial fluid, and meniscus, and the like, and any combination thereof. In various examples, musculoskeletal tissue(s) independently comprise cells and/or cell structures associated with cartilage, synovial membrane, ligaments, tendons, tendon sheaths, bursas, bone, synovial fluid, or meniscus, or the like, or any combination thereof. In various examples, the cells are abnormal cells (such as, for example, cancerous cells, or the like).

In various examples, imaging (such as, for example, fluorescence imaging) comprises: directing excitation electromagnetic radiation into the sample or the subject, thereby exciting at least one of the one or more fluorescent groups, if present or one or more radioisotope(s), if present, or a combination thereof resulting in emission or excitation and emission of electromagnetic radiation from the fluorescent group(s), if present, or the radioisotope(s), if present, or the combination thereof: detecting at least a portion of the emitted electromagnetic radiation, the detected electromagnetic radiation having been emitted by the fluorescent group(s), if present, or the radioisotope(s), if present, or the combination thereof; and processing signals corresponding to the detected electromagnetic radiation to provide one or more image(s) of the joint(s) or the portion thereof, the musculoskeletal tissue(s) or the portion thereof, or the combination thereof in the sample or the subject. In various examples, the electromagnetic radiation comprises one or more wavelength(s) from about 400 to about 1700 nm, including all 0.1 nm values and ranges therebetween. In various examples, the image(s) is/are fluorescence images. In various examples, the electromagnetic radiation is directed into a region of the subject or the sample, where the region is within the sample or the subject and the region comprises the one or more joint(s) or the portion thereof, one or more musculoskeletal tissue(s) or the portion thereof, or the combination thereof.

In various examples, electromagnetic radiation is directed into a region, where the region is within the sample or the subject and the region comprises the one or more joint(s) or the portion thereof, one or more musculoskeletal tissue(s) or the portion thereof, or the combination thereof. In various examples, a region comprises one or more joint(s) (or any portion thereof) (such as, for example, one or more cell(s) associated with the joint(s), extracellular matrix or the like), one or more musculoskeletal tissue(s) (or any portion thereof), or any combination thereof.

In various examples, the images are fluorescence images or the like. In various examples, a method comprises an intravital fluorescence imaging method, bioluminescence imaging, or the like.

In various examples, imaging comprises multiphoton imaging. In various examples, the multiphoton imaging comprises two-photon imaging, or three-photon imaging, or the like.

In various examples, the imaging is super-resolution imaging (such as, for example, optical super-resolution imaging). In various examples, at least a portion or all of the optical super-resolution image exhibits sub-diffraction limit resolution. In various examples, the image(s) resulting from the imaging are fluorescence image(s) or a sequence of fluorescence image(s) which can be processed to obtain an optical super-resolution image of the sample or portion thereof or the individual or a portion thereof.

In various examples, at least a portion or all the inorganic nanoimaging agent(s) is/are aluminosilicate nanoparticle(s), the aluminosilicate nanoparticle(s) independently comprising at least one organic dye molecule covalently bonded to the aluminosilicate network of the individual aluminosilicate nanoparticles and the imaging is optical super-resolution imaging.

In various examples, the imaging is an optical super-resolution microcopy method. Non-limiting examples of optical super-resolution microcopy methods include ground state depletion (GSD) microscopy, stochastic optical reconstruction microscopy (STORM), direct stochastic optical reconstruction microscopy (dSTORM), stimulated emission and depletion (STED), and photoactivated localization microscopy (PALM), and the like, and any combination thereof.

In various examples, a method further comprises an incubation time prior to imaging. In various examples, a therapeutic method further comprises an incubation time after administration and prior to imaging. In various examples, an imaging method further comprises an incubation time after contacting (e.g., administration) and prior to imaging. In various examples, a method further comprises an incubation time of 5 minutes to 24 hours, including all 0.1 minute values and ranges thereof prior to imaging.

In various examples, a method further comprises administration of one or more fluorescent nanoparticle(s) (which may be inorganic nanoimaging agent(s)) or the like and imaging those fluorescent nanoparticle(s) (which may be inorganic nanoimaging agent(s)). In various examples, the one or more fluorescent nanoparticle(s) (which may be inorganic nanoimaging agent(s)) have a distinct fluorescence emission spectrum (e.g., one or more distinct and observable fluorescence emission wavelength(s) or the like) from the initially administered inorganic nanoimaging agent(s) and/or inorganic nanoimaging agent(s).

In various examples, a method is a diagnosis method. In various examples, a therapeutic method or an imaging method further comprises a diagnosis method. In various examples, a diagnosis method comprises diagnosing (e.g., by a medical or veterinary professional or the like) or investigating a current or potential disease, a disease state, a condition, a disorder, a side effect, or any combination thereof (such as, for example, an inflammatory joint disease (such as, for example, arthritis (e.g., rheumatoid arthritis and osteoarthritis, and the like, or both), cancer (such as, for example, a cancer associated with a joint, a musculoskeletal tissue, or the like) or the like), which may be associated with one or more joint(s) or any portion thereof, one or more musculoskeletal tissue(s) or any portion thereof, or the combination thereof using the images of the sample or the subject.

In various examples, a method is a method of determining a presence or an absence of an analyte or a concentration of an analyte proximate to or within one or more joints(s) (or any portion thereof) (such as, for example, one or more cell(s) associated with the bone(s) (e.g., embedded cell(s) or the like) or the like), one or more tissue(s) (or any portion thereof) associated with the joint(s), for example, in a sample or in individual in a sample or a portion thereof or an individual or a portion thereof using one or more inorganic nanoimaging agent(s).

A method may comprise manipulation of one or more joint(s) or any portion thereof.

In various examples, one or more joints(s) (or any portion thereof) (such as, for example, one or more tissue(s) associated with the joints(s) (e.g., cartilage, synovial membrane, ligaments, tendons, tendon sheaths, bursas, bone, synovial fluid, and/or meniscus or the like) and/or cells (and/or cell structures) associated with tissues such as cartilage, synovial membrane, ligaments, tendons, tendon sheaths, bursas, bone, synovial fluid, and/or meniscus), one or more musculoskeletal tissue(s) (or any portion thereof), or any combination thereof is manipulated prior to and/or during and/or after contacting, administration, imaging, diagnosing, treating, or any combination thereof. Suitable methods of manipulation are known in the art.

Various inorganic nanotherapeutic imaging agents and inorganic nanoimaging agents can be used. In various examples, all of the inorganic nanotherapeutic imaging agent(s) and/or inorganic nanoimaging agents are substantially the same or the same. In various examples, two or more of the inorganic nanotherapeutic imaging agent(s) and/or inorganic nanoimaging agents are different (e.g., compositionally and/or structurally different). Non-limiting examples of suitable inorganic nanoimaging agents and inorganic nanoimaging agents are disclosed herein.

In various examples, inorganic nanotherapeutic agent(s) or inorganic nanoimaging agent(s) is/are chosen from nanoparticles, nanorings, nanocages, and the like, and any combination thereof. In various examples, inorganic nanotherapeutic agent(s) or inorganic nanoimaging agent(s) is/are Cornell Dots (such as, for example, Cornell Prime Dots (C′ Dots, aC′ Dots, or the like) or the like). In various examples, an inorganic nanotherapeutic agent or an inorganic nanoimaging agent further comprises a plurality of polyethylene glycol (PEG) groups disposed on (e.g., covalently bonded or non-covalently bonded) at least a portion of a surface or all of the surfaces (which may be pore surface(s), exterior (non-pore) surface(s), or any combination thereof) of the inorganic nanoimaging agent (e.g., nanoparticle, nanoring, nanocage, or the like) (which may be referred to as PEGylated inorganic nanoimaging agent).

In various examples, an inorganic nanotherapeutic agent comprises one or more targeting group(s) and one or more therapeutic groups(s), where each group is independently disposed (e.g., covalently bonded or non-covalently bonded) to a surface of the inorganic nanotherapeutic agent. In various examples, an inorganic nanotherapeutic agent further comprises one or more fluorescent group(s), one or more sulfur atom group(s), one or more radioisotope group(s), one or more heavy atom group(s), or any combination thereof, where each group is disposed (e.g., covalently bonded or non-covalently bonded) to a surface of the inorganic nanotherapeutic agent.

In various examples, an inorganic nanoimaging agent comprises one or more fluorescent group(s) and one or more targeting group(s), where each group is independently disposed on (e.g., covalently bonded or non-covalently bonded to) a surface of the inorganic nanotherapeutic agent or disposed in (e.g., covalently bound to) the matrix (such as, for example, the silica matrix, the aluminosilicate matrix, or the like) of the inorganic nanoimaging agent. In various examples, an inorganic nanotherapeutic agent further comprises one or more sulfur atom group(s), one or more radioisotope group(s), one or more heavy atom group(s), one or more therapeutic group(s), or any combination thereof, where each group is disposed (e.g., covalently bonded or non-covalently bonded) to a surface of the inorganic nanotherapeutic agent.

Non-limiting examples of silica-poly (ethylene glycol)(PEG) core shell particles include:

| Core | |
|---|---|
| particles | Description |
| C Dots | Fluorescent core-shell silica nanoparticles (C Dots) covalently incorporate |
| | an organic fluorescent dye into the center of the particle, increasing its |
| | brightness and photostability for imaging and diagnostic probe |
| | applications. The sol-gel synthesis of C′Dots in organic solvents can create |
| | particles ranging in size from 5-30 nm. |
| C′Dots | Fluorescent core-shell C′Dots differ from the previous generation of C |
| | Dots due to their “one-pot” synthesis in water and brush-like poly(ethylene |
| | glycol) PEG coating which minimizes protein corona formation around the |
| | particles and increases bioinertness and stability of the particles. C′Dot |
| | synthesis additionally facilitates the introduction of multiple different |
| | surface functional groups via post-PEGylation surface modification by |
| | insertion (PPSMI). |
| aC′Dots | Fluorescent core-shell aC′Dots are an aluminum-containing modification of |
| | C′Dots. The core of aC′Dots contains silica and aluminum at variable |
| | ratios, including a 92:8 ratio, such that aluminum is fourfold coordinated in |
| | aC′Dots which enhances aluminosilicate matrix rigidity and thus, dye |
| | brightness & stability. aC′Dots also uniquely display stochastic dye |
| | blinking (e.g., in the near-infrared, NIR), which enables STORM based |
| | optical super-resolution microscopy (SRM) without specific imaging |
| | buffers and second lasers. |
| pC′Dots | Fluorescent core-shell pC′Dots are a phophonate-containing modification |
| | of C′Dots. The core of the pC′Dots contains silica and phosphonate, the |
| | ratio of which can be optimized for increased enhancement of dye |
| | brightness. This ratio can also be used to tune final particle size, e.g., from |
| | 3.5-7 nm, and the addition of phosphonate enhances the intrinsic metal |
| | chelating abilities of C′Dots. Prior to PEGylation, additional silica shells |
| | can be grown to increase particle core size. This synthesis also enables |
| | functionalization via PPSMI. |

Non-limiting examples of nanoparticles that can be used as inorganic nanotherapeutic agents or inorganic nanoimaging agents and methods of making and using same are described in International Application No. PCT/US2016/030752 (Title: Ultrasmall Nanoparticles and Methods of Making and Using Same; filed May 4, 2016), International Application No. PCT/US2018/026980 (Title: Sulfur- or Heavy Atom-Containing Nanoparticles, Methods of Making Same, and Uses Thereof, filed Apr. 10, 2018), International Application No. PCT/US2019/026411 (Title: Inorganic Nanocages, and Methods of Making and Using Same; filed Apr. 8, 2019), International Application No. PCT/US2020/028372 (Title: Functionalized Silica Nanorings, Methods of Making Same, and Uses Thereof: filed Apr. 15, 2020), and U.S. patent application Ser. No. 17/501,854 (Title: Super Resolution Optical Microscopy Using Aluminosilicate Nanoparticles; filed Oct. 14, 2021) and are each incorporated herein for its disclosure of nanoparticles, nanorings, nanocages, and the like and methods of making same and imaging methods and treatment methods.

Inorganic nanotherapeutic imaging agents and inorganic nanoimaging agents have various sizes. In various examples, inorganic nanotherapeutic agent(s) and/or inorganic nanoimaging agent(s) comprise a longest linear dimension (which may be a diameter, such as, for example, a hydrodynamic diameter, a TEM diameter, or the like) of about 2 nanometers (nm) to about 1 micrometer (micron), including all 0.1 nm values and ranges therebetween (e.g., about 2 nm to about 100 nm). In various examples, inorganic nanotherapeutic agent(s) and/or inorganic nanoimaging agent(s) comprise a longest linear dimension (which may be a diameter, such as, for example, a hydrodynamic diameter, a TEM diameter, or the like) or the like) of about 2 nanometers (nm) to about 10 nm, including all 0.1 nm values and ranges therebetween.

The size of an inorganic nanotherapeutic imaging agent or agents or an inorganic nanoimaging agent or agents can be determined by methods known in the art. In various examples, the size (which may be an average size) of an inorganic nanotherapeutic imaging agent or agents or an inorganic nanoimaging agent or agents is determined by dynamic light scattering, transmission electron microscopy, or the like, or a combination thereof.

An inorganic nanotherapeutic agent or an inorganic nanoimaging agent can comprise various fluorescent groups. In the case where the inorganic nanotherapeutic agent or an inorganic nanoimaging agent comprises a plurality of fluorescent groups, all the fluorescent groups may be the same or two or more of the fluorescent groups may be different (e.g., structurally different).

In various examples, a fluorescent group comprises or is a small molecule fluorescent groups (such as, for example, a dye group(s) or the like). In various examples, a fluorescent group comprises one or more dye groups(s). In various examples, a dye group comprises or is an organic dye group or a combination of organic dye group(s). In various examples, a fluorescent group comprises or is one or more fluorescent dye groups(s). In various examples, at least a portion or all of the fluorescent group(s) (such as, for example, dye group(s) (e.g., organic dye groups (which may be independently formed from an organic dye or the like) is/are fluorescent dye groups(s), fluorescent peptide groups(s), fluorescent protein groups(s), or the like, or any combination thereof. In various examples, at least a portion or all of fluorescent dye group(s) is/are near infrared (NIR) dye groups or the like. In various examples, the organic dye group(s) is/are chosen from cyanine dyes, rhodamine dyes (e.g., carborhodamine dyes and the like), coumarin dyes, boron-dipyrromethene (BODIPY) dyes, xanthene dyes, eosin dyes, carbopyronine dyes, methylene blue, fluorescein, Acridine Orange, structural derivatives thereof, or any group derived therefrom, or any combination thereof. In various examples, a fluorescent group comprises a fluorophore or comprises a fluorophore group.

In various examples, a fluorescent group comprises a fluorophore or comprises a fluorophore group. In various examples, a fluorophore is or a fluorophore group is formed from a fluorescent aromatic or heteroaromatic compound or compounds, such as, for example, a pyrene, an anthracene, a naphthalene, an acridine, a stilbene, an indole or benzindole, an oxazole, benzoxazole, a thiazole, a benzothiazole, a 4-amino-7-nitrobenz-2-oxa-1,3-diazole (NBD), a cyanine, a carbocyanine, a carbostyryl, a porphyrin, a salicylate, an anthranilate, an azulene, a perylene, a pyridine, a quinoline, a coumarin (including hydroxycoumarins and aminocoumarins and fluorinated derivatives thereof), and like or any combination thereof. Non-limiting examples of compounds are disclosed in U.S. Pat. Nos. 5,830,912; 4,774,339; 5,187,288; 5,248,782; 5,274,113; 5,433,896; 4,810,636; and 4,812,409, the disclosure of which with regard to fluorophores and fluorescent aromatic or heteroaromatic compounds is incorporated herein by reference.

An inorganic nanotherapeutic agent or an inorganic nanoimaging agent can comprise various radioisotope groups. In the case where the inorganic nanotherapeutic agent or an inorganic nanoimaging agent comprises a plurality of radioisotope groups, all the radioisotope groups may be the same or two or more of the radioisotope groups may be different (e.g., structurally different). Non-limiting examples of radioisotopes include ¹²⁴I, ⁸⁹Zr, ¹¹C, ¹⁵O, ¹⁸F, ⁶⁴Cu, and ⁶⁸Ga, and the like, and any combination thereof.

An inorganic nanotherapeutic agent or an inorganic nanoimaging agent can comprise various heavy atom groups. In various examples, incorporation of a heavy atom in an inorganic nanotherapeutic agent or an inorganic nanoimaging agent leads to spin-orbit coupling with fluorophore group(s) (such as, for example, dyes or the like) of the inorganic nanotherapeutic agent or the inorganic nanoimaging agent, in turn leading to inter-system crossing and formation of triplet-state excitation states of the inorganic nanotherapeutic agent or the inorganic nanoimaging agent. In the case where the inorganic nanotherapeutic agent or an inorganic nanoimaging agent comprises a plurality of heavy atom groups, all the heavy atom groups may be the same or two or more of the heavy atom groups may be different (e.g., structurally different).

In various examples, the heavy atom group(s) are neutral or charged heavy atoms/heavy atom groups. In various examples, the heavy atom group(s) is/are chosen from iodine atom, bromine atom, metal ions (such as, for example, Au ions, Ag ions, Pb ions, Ti ions, Bi ions. Pt ions, In ions, Sn ions, Sb ions or Pd ions, and the like), and structural derivatives thereof, and any group derived therefrom, or any combination thereof.

An inorganic nanotherapeutic agent or an inorganic nanoimaging agent can comprise various targeting groups. In the case where the inorganic nanotherapeutic agent or an inorganic nanoimaging agent comprises a plurality of targeting groups, all the targeting groups may be the same or two or more of the targeting groups may be different (e.g., structurally different)

Non-limiting examples of targeting group(s) include tissue targeting groups, tissue-resident cell targeting groups, groups with affinity to specific tissue types (e.g., electrostatic interaction or the like) (such as, for example, cationic peptides and the like), and any combination thereof. Non-limiting examples of targeting groups include integrins, transmembrane proteins (such as, for example, ion channels, connexins, pannexins, purinergic channels, and the like), intracellular signaling proteins, and the like, and any combination thereof.

Non-limiting examples of targeting moieties for cartilage extracellular matrix include targeting moieties formed from:

| | Molecule | |
|---|---|---|
| Target | Category | Examples |
| Collagen-II | Peptides | WYRGRL (SEQ ID NO: 1), DPHFHL (SEQ ID NO: 2), |
| | | RVMLVR (SEQ ID NO: 3), |
| | | GDYVIDWNFIEW (SEQ ID NO: 11), TVGSFFVEWMMH |
| | | (SEQ ID NO: 12), DIGGWFVEWSLA (SEQ ID NO: 13), |
| | | DWGYFSWAYDSA (SEQ ID NO: 14), DWYTVSWLTDSN |
| | | (SEQ ID NO: 15), DAYWHPVWVHDP (SEQ ID NO: 16), |
| | | DLQYWYPIWDTH (SEQ ID NO: 17), HVYQKPSYWWYP |
| | | (SEQ ID NO: 18), TWHFVDFSADTH (SEQ ID NO: 19), |
| | | DYFTLDFTFDSW (SEQ ID NO: 20), NQVYFHYFDLDF (SEQ |
| | | ID NO: 21), SPWWLWKAHNEA (SEQ ID NO: 22), |
| | | EVENHYIQYSTE (SEQ ID NO: 23), LPGMELFWNVAN (SEQ |
| | | ID NO: 24), DTFVFGSSKWRA (SEQ ID NO: 25), |
| | | SNNMRAPVNEIY (SEQ ID NO: 26) |
| | Antibodies | mAbCII, anti-CII antibody, anti-CII (e.g. LSBio: LS-C825911- |
| | | GOS10-100) and anti-CCP antibodies (e.g. labcorp: 164914) |
| | Ab- | Avimer M26 |
| | mimetic | |
| | Antibody | e.g. anti-ROS-modified CII scFv |
| | fragments | |
| COMP | Antibodies | e.g. Proteintech: 28369-1-AP |
| Hyaluronan | Peptides | e.g. Abcam: ab199378 |
| | Antibodies | e.g. Invitrogen: PA1-85561 |
| Aggrecan | Cationic | (RRAAAA)3RR (SEQ ID NO: 27), |
| | peptides | RRRR(AARRR)3R (SEQ ID NO: 28), |
| | | (ARRRAARA)4 (SEQ ID NO: 29), |
| | | (RRRRR)4 (SEQ ID NO: 30), |
| | | (AK)7ANANAN (SEQ ID NO: 31), |
| | | (AK) 7AFAFAF (SEQ ID NO: 32) |
| | Cysteine | GSISIGIKCSPSIDLCEGQCRIRKYFTGYCSGDTCHCSG |
| | | (SEQ ID NO: 33), |
| | dense | GSSCARPRENCNRMNILCCRGECVCPTFGDCFCYGD (SEQ |
| | | ID NO: 34), |
| | peptides | GSGVPINVKCRGSRDCLDPCKKAGMRFGKCINSKCHCTP |
| | | (SEQ ID NO: 35), |
| | | GSGVPINVRCRGSRDCLDPCRRAGMRFGRCINSRCHCTP |
| | | (SEQ ID NO: 36), |
| | | GSGVPINVRSRGSRDSLDPSRRAGMRFGRSINSRSHSTP |
| | | (SEQ ID NO: 37), |
| | | GSVRIPVSCRHSGQCLRPCRDAGMRFGRCMNGRCDCTPR |
| | | (SEQ ID NO: 38), |
| | | GSQVQTNVRCQGGSCASVCRREIGVAAGRCINGRCVCYR |
| | | N (SEQ ID NO: 39) |
| | Polymers/ | Poly-beta-amino-esters, Poly(amidoamine)(PAMAM) dendrimer |
| | Dendrimers | |
| Bone | Peptide | pVTK peptide |

Non-limiting examples of targeting moieties for tissue-resident cells include targeting moieties formed from:

| | Molecule | |
|---|---|---|
| Target cell | Category | Examples |
| Chondrocytes | Peptides | DWRVIIPPRPSA (SEQ ID NO: 40), p5RHH, |
| | | RLDPTSYLRTFW (SEQ ID NO: 41), |
| | | HDSQLEALIKFM (SEQ ID NO: 42), |
| | | vasoactive intestinal peptide |
| | Antibodies | Anti-CD44 |
| OA Chondrocytes | Antibodies | IL-1Ra |
| Activated Macrophages | Other | folic acid-modified hyaluronic acid, |
| | | anti-CD16/32 antibody, |
| | | dextran sulfate |
| Fibroblast-like | Peptides | SFHQFARATLAS (HAP-1) (SEQ ID NO: 43), |
| Synoviocytes | | tuftsin peptide, RGD, folic acid, αvβ3- |
| | | targeted, macrophage-derived microvesicle |
| | | proteins, red blood cell membrane proteins |
| Synovial micro vascular | Peptides | CKSTHDRLC (SEQ ID NO: 44), |
| endothelium (MVE) | | CKPFDRALC (SEQ ID NO: 45) |

An inorganic nanotherapeutic agent or inorganic nanoimaging agent may target a specific structure in a sample or an individual. Without intending to be bound by any particular theory, it is considered that a targeting group or targeting groups direct(s) an inorganic nanoimaging agent to a specific target in a sample or in an individual. In various examples, an inorganic nanoimaging agent targets one or more tissue(s) in one or more articulating joint(s) (such as, for example, articular cartilage, bone, meniscus, tendon, ligament, or synovium, or the like, or any combination thereof). In various examples, an inorganic nanoimaging agent targets specific cells (or a portion thereof). In various examples, an inorganic nanoimaging agent targets tendon cells, ligament cells, meniscus cells, synovial cells, macrophages, osteocytes (or a portion thereof), bone marrow cells (such as, for example, stem cells (e.g., hematopoietic stem cells, mesenchymal stem cells, and the like), blood cells (e.g., red blood cells, leukocytes, white blood cells, platelets, and the like), lymphocytes, fibroblasts, bone-lining cells, osteoblasts, osteoclasts, chondrocytes, nerve cells, blood vessel cells (e.g., sinusoids, endothelial cells, smooth muscle cells, or the like), or the like, or any combination thereof, or a portion thereof.

An inorganic nanotherapeutic agent or an inorganic nanoimaging agent can comprise various therapeutic groups. An inorganic nanotherapeutic agent or an inorganic nanoimaging agent can comprise various therapeutic groups. In the case where the inorganic nanotherapeutic agent or an inorganic nanoimaging agent comprises a plurality of therapeutic groups, all the targeting groups may be the same or two or more of the therapeutic groups may be different (e.g., structurally different).

The therapeutic group of an inorganic nanotherapeutic agent or an inorganic nanoimaging agent is not particularly limited. It is considered that any therapeutic agent (e.g., drug, biologic agent (such as, for example, protein, peptide, or the like), or the like) that is used for treatment of a disease, a disease state, a condition, a disorder, a side effect, or any combination thereof (such as, for example, an inflammatory joint disease (such as, for example, arthritis (e.g., rheumatoid arthritis and osteoarthritis, and the like, or both), cancer (such as, for example, a cancer associated with a joint, a musculoskeletal tissue, or the like) or the like) can be delivered (e.g., specifically delivered) to an individual as a therapeutic group of an inorganic nanotherapeutic agent or an inorganic nanoimaging agent.

Non-limiting examples of therapeutic groups include anti-inflammatory therapeutic groups, catabolic groups, anti-catabolic therapeutic groups, anabolic therapeutic groups, pro-anabolic groups, bioenergetic groups, viscosupplement groups, corticosteroid groups, steroid groups, antioxidant groups, gene therapy groups, nucleotide groups (such as, for example, RNA groups (e.g., mRNA groups, siRNA groups, and the like) and the like), and anu combination thereof.

Non-limiting examples of therapeutic moieties (e.g., functional groups) include therapeutic moieties formed from;

| Therapeutic Category | Therapeutic Target | Examples |
|---|---|---|
| Anti-inflammatory | AAV9 | Follistatin |
| | Il-1B | HDV-NFκB-Il-Ira, Canakinumab, |
| | | Anakinra, Diacerin, Capparis |
| | | spinosa L., Euptalin, Genistein, |
| | | Quercetin, glucosamine, chondroitin, |
| | | palmitoylethanolamide, curcumin |
| | Il-1, 6, 8 | Anakinra, AMG 108, ABT 981, |
| | | Diacerin, Resveratrol |
| | TNF-a | Infliximab, adalimumab, etanercept |
| | PGE₂ | Moraxanthin, Morin |
| | NGF | Monoclonal antibodies against NGF |
| | | (e.g. Tanezumab, Fluranumab, |
| | | Fasinumab) |
| | COX Enzymes | Ibuprofen, Naproxen, Celecoxib |
| | | Natural compounds: Boswellia |
| | | serrata, Arnica montana, Apis |
| | | mellifera, Psoralea |
| | | corylifolia, Rhizome coptidis, Betulae |
| | | cortex, Harpagophytum |
| | | procumbens, Phellodendron |
| | | amurense, Symphytum officinalis, |
| | | epigallocatechin gallate, and Withania |
| | | somnifera, |
| | | apigenin, cirsimarin, cirsimaritin, |
| | | hispidulin, jaceosidin, wogonin, and |
| | | luteolin, Seomae mugwort |
| | Multiple targets | Dexamethasone, ROS-scavening |
| | | materials (e.g. vitamin E), miRNA- |
| | | 122, forskolin, periostin-regulation via |
| | | matrikine Link N (e.g. |
| | | DHLSDNYTLDHDRAIH), |
| | | piperlongumine, osteoactivin (e.g. |
| | | recombinant or related-peptide), A2- |
| | | macroglobulin, deferoxamine, irisin, |
| | | metformin |
| Anti-catabolic | Proteases and pro- | Inhibitors of MMP-1,3,7,9,13, |
| | protein convertases | aggrecanases (ADAMTS-4, |
| | | ADAMTS-5), cathepsins, PACE4, |
| | | PHLPP, SHP2, and others |
| | MMP-1,3,7,9,13, | RNA interference technology (e.g. |
| | ADAMTS-4, | siRNA) for Mmp13, Adamts5, HIF-2a, |
| | ADAMTS-5, | Epas1 |
| | cathepsins, PACE4 | Antibodies: 2D3, 2D5 and 2D11, 2B9 |
| | | (https://doi.org/10.1042/BJ20150758), |
| | | PG-11800, ALS 1-0635, CL82198, |
| | | GLPG1972, M6495, MIV-711 |
| | Hyaluronidase | Heparin, Heparin sulfate, Kallikrein, |
| | | salicylates, indomethacin, disodium |
| | | cromoglycate, tranilast, flavonoids, |
| | | tannins, hydrangenols, curcumins, |
| | | glycyrrhizin, cinnamic acid, inter- |
| | | alpha-inhibitor |
| | Glucocorticoid | Salts of dexamethasone, triamcinolone |
| | receptors | and prednisone |
| Anabolic | AV | RHEB gene (NM_005614.3) |
| | PRG4 | HDV-EF1-PRG4 |
| | TGF-B1, 2, 3 | Peptides: HSNGLPL |
| | | Cleavable conjugates: LAP-MMP- |
| | | IFN-beta, LAP-TIMP-3 fusion |
| | | proteins, LAP- (VIP, αMSH or |
| | | γ3MSH), LAP-( TGF-β, |
| | | erythropoietin, IL-1ra, IL-10, IL-4, |
| | | BMP-7, IGF1 and IL-17) |
| | | Losartan |
| | | Antibodies and antibody fragments |
| | Growth factors | IGF-1, IGF-2, FGF-2, BMP-2, BMP-4, |
| | | BMP-7, PDGF |
| | Heparin Sulfate | Peptides: |
| | | KRKKKGKGLGKKRDPSLRKYK |
| | | (SEQ ID NO: 46), LRSRTKIIRIRH |
| | | (SEQ ID NO: 47), MPRRRRIRRRQK |
| | | (SEQ ID NO: 48) |
| | | Heparin mimetics: (e.g. modified |
| | | polysaccharides, synthetically sulfated |
| | | oligosaccharides, oligosaccharide- |
| | | aglycone conjugates and non- |
| | | carbohydrate-based sulfated mimetics) |
| | Osteoblasts and | Bisphosphonates: Alendronate, |
| | chondrocytes | Zoledronate, Zoledronic acid, |
| | | Clodronate |
| | | Antiosteoporotic: Strontium ranelate, |
| | | Others: Kartogenin, KA34, LNA043, |
| | | Senolytics (e.g. Navitoclax), |
| | | senomorphics (e.g. UBX0101) |
| | Multiple targets | Metformin |
| Bioenergetic (e.g. | mtDNA | SSRNA, siRNA, mRNA for activation |
| mitochondria (MT)) | | of AMPK/SIRT1/3/PGC-1α, |
| | | AMPK/SIRT3/SOD2, and |
| | | AMPK/SIRT3/Parkin/PINK1 |
| | | pathways; |
| | | upregulate: OPA1, Mfn1, and Mfn2 |
| | | downregulate: Drp1 and Dnm2 |
| | Therapeutic molecules | Melatonin, Resveratrol, |
| | (antioxidants, | Dihydromyricetin, Apple procyanidins, |
| | apoptotic inhibitors, | Zinc, SIRT3 activator, Quercetin, |
| | enhance MT | Puerarin, LRWXG, Ginsenoside Rg1, |
| | dynamics) | Chondroitin sulfate, taurine, Diallyl |
| | | Disulfide |
| | SS peptides | D-Arg-Dmt-Lys-Phe-NH₂ (i.e. SS-31 |
| | | or MTP-131 or elamipretide), |
| | | SS-01 (H-Tyr-D-Arg-Phe-Lys-NH₂ |
| | | (SEQ ID NO: 49)), |
| | | SS-02 (H-Dmt-D-Arg-Phe-Lys-NH₂), |
| | | SS-19, SS-20 (H-Phe-D-Arg-Phe-Lys- |
| | | NH₂ (SEQ ID NO: 50)) |
| Viscosupplements | HA/PA and HA/PM | Hyaluronan and Hyaluronic Acid of |
| | | varying molecular weights |
| | Proteoglycan-4 | Natural and synthetic PRG4 and |
| | | lubricin analogues |

In various examples, a therapeutic group is formed from a drug (such as, for example, a small molecule drug, or the like) typically used to treat a current or potential disease, disease state, condition, disorder, side effect, or any combination thereof (such as, for example an inflammatory joint disease (such as, for example, arthritis (e.g., rheumatoid arthritis and osteoarthritis, and the like, or both), cancer (such as, for example, a cancer associated with a joint, a musculoskeletal tissue, or the like) or the like). In various examples, a therapeutic group is formed from a disease-modifying anti-rheumatic drug (such as, for example, DMARD or the like).

In various examples, an inorganic nanotherapeutic agent or agents or an inorganic nanoimaging agent or agents is present in a composition. Non-limiting examples of compositions are provided herein.

An inorganic nanotherapeutic agent or agents or inorganic nanoimaging agent and/or agents or composition or composition(s) can be administered by various routes.

In various examples, inorganic nanotherapeutic agent(s) and/or inorganic nanoimaging agent(s) or composition(s) is/are administered proximate to joint(s) (or any portion thereof) (such as, for example, one or more cell(s) associated with the joint(s) (e.g., extracellular matrix or the like) or the like), musculoskeletal tissue(s) (or any portion thereof), or any combination thereof of an individual. In various examples, administration comprises a single dose or multiple doses. In various examples, the administration (which may be a single dose or multiple doses) is subcutaneous administration, intravenous administration, intraarticular administration, infrapatellar fat pad administration, intramarrow administration, or the like.

In various examples, an administration administers about 1 microliter to about 10 milliliters, including all 0.1 microliter values and ranges therebetween. In various examples, an administration is subcutaneous administration and about 1 microliter to about 50 microliters, including all 0.1 microliter values and ranges therebetween, is administered. In various examples, an administration is intraarticular administration and about 1 microliter to about 10 milliliters, including all 0.1 microliter values and ranges therebetween, is administered.

In various examples, administration of the inorganic nanotherapeutic agent(s) and/or inorganic nanoimaging agent(s) results in delivery of a targeting group or targeting groups (or an unconjugated structural analog of the therapeutic group(s)) to the individual.

In various examples, inorganic nanotherapeutic agent(s) and/or inorganic nanoimaging agent(s) comprise a longest linear dimension (which may be a diameter, such as, for example, a hydrodynamic diameter, a TEM diameter, or the like) of about 2 nanometers (nm) to about 1 micrometer (micron), including all 0.1 nm values and ranges therebetween (e.g., about 2 nm to about 100 nm), and the administration is intravenous. In various examples, inorganic nanotherapeutic agent(s) and/or inorganic nanoimaging agent(s) comprise a longest linear dimension (which may be a diameter, such as, for example, a hydrodynamic diameter, a TEM diameter, or the like) of about 2 nanometers (nm) to about 10 nm, and the administration is subcutaneous.

In various examples, the inorganic nanoimaging agent(s) (e.g., the engineered nanoparticle(s) or the like) comprise a longest linear dimension (such as, for example, a diameter or the like) (which may be an average longest linear dimension (such as, for example, an average diameter or the like) of about 2 nanometers (nm) to about 1 micrometer (micron), including all 0.1 nm values and ranges therebetween, and the administration is intravenous. In various examples, the inorganic nanoimaging agent(s) (e.g., the engineered nanoparticle(s) or the like) comprise a longest linear dimension (such as, for example, a diameter or the like) (which may be an average longest linear dimension (such as, for example, an average diameter or the like) of about 2 nanometers (nm) to about 10 nm, including all 0.1 nm values and ranges therebetween, and the administration is subcutaneous.

The size of inorganic nanotherapeutic imaging agent(s) and/or inorganic nanoimaging agent(s) can be determined by methods known in the art. In various examples, the size (which may be an average size) of an inorganic nanotherapeutic imaging agent or agents or an inorganic nanoimaging agent or agents is determined by dynamic light scattering, transmission electron spectroscopy (TEM), or the like, or a combination thereof.

An inorganic nanotherapeutic or an inorganic nanoimaging agent may exhibit desirable residence time in a subject. In various examples, at least a portion of the inorganic nanotherapeutic agent(s) and/or an inorganic nanoimaging agent(s) reside(s) in into one or more joints(s) (or any portion thereof) (such as, for example, one or more cell(s) associated with the bone(s) (e.g., embedded cell(s) or the like) or the like), one or more musculoskeletal tissue(s) or any portion thereof, or any combination thereof, or any combination thereof in the subject or in a sample for at least 14 days, at least 21 days, at least 28 days, or at least 3 months. In various examples, at least a portion of the inorganic nanotherapeutic agent(s) and/or an inorganic nanoimaging agent(s) reside(s) in one or more joints(s) (or any portion thereof), one or more musculoskeletal tissue(s) (or any portion thereof), or any combination thereof in the subject for at least 2 times longer, at least 5 times greater, at least 10 times greater than a corresponding therapeutic agent corresponding to the therapeutic group(s) (e.g., the therapeutic agent is an unconjugated structural analog of the therapeutic group).

An inorganic nanotherapeutic agent or an inorganic nanoimaging agent may exhibit desirable penetration into (and, in various examples, desirable retention in) into one or more joints(s) (or any portion thereof) (such as, for example, one or more cell(s) associated with the bone(s) (e.g., embedded cell(s) or the like) or the like), one or more musculoskeletal tissue(s) (or any portion thereof), or any combination thereof of a sample or an individual. In various examples, at least a portion of the inorganic nanotherapeutic agent(s) and/or the inorganic nanoimaging agent(s) penetrates(s) (e.g., prior to any substantial clearing of the inorganic nanotherapeutic agent(s) and/or or an inorganic nanoimaging agent(s)) at least 25% or more, at least 30% or more, at least 40% or more, at least 50% or more, at least 75% or more, or completely through a depth of the joints(s) (or any portion thereof) (such as, for example, one or more cell(s) associated with the bone(s) (e.g., embedded cell(s) or the like) or the like), musculoskeletal tissue(s) (or any portion thereof), or any combination thereof.

In various examples, at least a portion of the inorganic nanotherapeutic agent(s) reside(s) in one or more joint(s) chosen from ankle joints, knee joints, hip joints, finger joints, wrists, elbows, and shoulder joints, and the like, and any combination thereof and/or musculoskeletal tissue(s) chosen from cartilage, synovial membrane, ligaments, tendons, tendon sheaths, bursas, bone, synovial fluid, and meniscus, and the like, any combination thereof. In various examples, the musculoskeletal tissue(s) independently comprise cells and/or cell structures associated with cartilage, synovial membrane, ligaments, tendons, tendon sheaths, bursas, bone, synovial fluid, or meniscus, or the like, or any combination thereof and at least a portion of the inorganic nanotherapeutic agent(s) reside(s) in the cells and/or the cell structures. Without intending to be bound by any particular theory it is considered that the residence time of inorganic nanotherapeutic agent can provide desirable treatment of a subject.

An inorganic nanotherapeutic agent and/or inorganic nanoimaging agent may be taken up by cells of a subject. In various examples, at least a portion of the inorganic nanotherapeutic agent(s) and/or inorganic nanoimaging agent(s) is taken up by and/or reside in chondrocytes, meniscus cells, ligament cells, tendon cells, lymphocytes, bone cells, or synovial cells, or any combination thereof of an individual. Without intending to be bound by any particular theory it is considered that the uptake of inorganic nanotherapeutic agent by certain cells can provide desirable treatment of a subject.

In an aspect, the present disclosure provides inorganic nanotherapeutic agents and inorganic nanoimaging agents. In various examples, an inorganic nanotherapeutic agent or a inorganic nanoimaging agent is configured or suitable for use in a method of the present disclosure. Non-limiting examples of compositions are described herein.

In various examples, the inorganic nanoimaging agent(s) is/are chosen from nanoparticles, nanorings, nanocages, and the like, and any combination thereof. In various examples, an inorganic nanoimaging agent further comprises a plurality of polyethylene glycol (PEG) groups disposed on at least a portion of a surface or all of the surfaces of the inorganic nanoimaging agent nanoparticle.

In various examples, an inorganic nanotherapeutic agent is a nanoparticle, a nanoring, a nanocage or the like comprising a silica core or matrix, a plurality of PEG groups and therapeutic group(s) chosen from IL-1 Ra (receptor agonist), TNF-a (alpha), or the like, or any combination thereof (which may independently be disposed on a surface of the inorganic nanotherapeutic agent or a PEG group comprises the therapeutic group). In various examples, the inorganic nanotherapeutic agent comprises a size (e.g., a longest linear dimension (which may be a diameter, such as, for example, a hydrodynamic diameter, a TEM diameter, or the like) of about 2 nanometers (nm) to about 10 nm, including all 0.1 nm values and ranges therebetween. In various examples, the inorganic nanotherapeutic agent further comprises one or more targeting group(s) (such as, for example, Type II collagen targeting peptides (e.g., WYRGL (SEQ ID NO. 1) or the like) or the like), one or more fluorophore group(s), one or more sulfur atom group(s), one or more radioisotope group(s), one or more heavy atom group(s), or the like, or any combination thereof, or any combination thereof.

In various examples, an inorganic nanoimaging agent is a nanoparticle, a nanoring, a nanocage or the like comprising a silica core or matrix, a plurality of PEG groups, fluorophore group(s) (such as, for example, Cy5 groups or the like), one or more targeting group(s) (such as, for example, Type IL collagen targeting peptides (e.g., WYRGL (SEQ ID NO. 1) or the like) or the like) and the like (which may independently be disposed on a surface of the inorganic nanoimaging agent or a PEG group comprises the targeting group). In various examples, the inorganic nanoimaging agent further comprises therapeutic group(s) chosen from IL-1 Ra (receptor agonist), TNF-a (alpha), or the like, or any combination thereof (which may independently be disposed on a surface of the inorganic nanoimaging agent or a PEG group comprises the therapeutic group). In various examples, the inorganic nanoimaging agent comprises a size (e.g., a longest linear dimension (which may be a diameter, such as, for example, a hydrodynamic diameter, a TEM diameter, or the like) of about 2 nanometers (nm) to about 10 nm, including all 0.1 nm values and ranges therebetween. In various examples, the inorganic nanoimaging agent further comprises one or more sulfur atom group(s), one or more radioisotope group(s), one or more heavy atom group(s), or the like, or any combination thereof, or any combination thereof.

In an aspect, the present disclosure provides compositions. In various examples, a composition comprises one or more inorganic nanotherapeutic agent(s) and/or one or more inorganic nanoimaging agent(s) of the present disclosure. Non-limiting examples of compositions are described herein.

In various examples, a composition is suitable for administration to an individual. In various examples, a composition is suitable for subcutaneous administration, intravenous administration, intraarticular administration, infrapatellar fat pad administration, intramarrow administration, or the like. In various examples, a composition is suitable for injection into a joint.

In various examples, a composition comprises inorganic nanotherapeutic agent(s) and/or inorganic nanoimaging agent(s) (e.g., engineered nanoparticle(s) or the like) at a concentration of 10 nanomolar to 100 micromolar, including all 0.1 micromolar values and ranges therebetween (e.g., 1 micromolar to 5 micromolar or 5 micromolar to 100 micromolar).

In various examples, the inorganic nanoimaging agent(s) (e.g., the engineered nanoparticle(s) or the like) comprise a longest linear dimension (such as, for example, a diameter or the like) (which may be an average longest linear dimension (such as, for example, an average diameter or the like) of about 2 nanometers (nm) to about 1 micrometer (micron), including all 0.1 nm values and ranges therebetween, and the administration is intravenous or intraarticular. In various examples, the inorganic nanoimaging agent(s) (e.g., the engineered nanoparticle(s) or the like) comprise a longest linear dimension (such as, for example, a diameter or the like) (which may be an average longest linear dimension (such as, for example, an average diameter or the like) of about 2 nanometers (nm) to about 10 nm, including all 0.1 nm values and ranges therebetween, and the administration is subcutaneous or intravenous.

Inorganic nanotherapeutic imaging agents and inorganic nanoimaging agents can have various sizes. In various examples, about 70% or more, about 80% or more, about 90% or more, about 95% or more, about 98% or more, about 99% or more, about 99.5% or more, about 99.9% or more, or about 100% of the inorganic nanotherapeutic agent(s) and/or inorganic nanoimaging agent(s) in a composition comprise a longest linear dimension (which may be a diameter, such as, for example, a hydrodynamic diameter, a TEM diameter, or the like) of about 2 nanometers (nm) to about 1 micrometer (micron), including all 0.1 nm values and ranges therebetween (e.g., about 2 nm to about 100 nm). In various examples, about 70% or more, about 80% or more, about 90% or more, about 95% or more, about 98% or more, about 99% or more, about 99.5% or more, about 99.9% or more, or about 100% of the inorganic nanotherapeutic agent(s) and/or inorganic nanoimaging agent(s) comprise a longest linear dimension (which may be a diameter, such as, for example, a hydrodynamic diameter, a TEM diameter, or the like) of about 2 nanometers (nm) to about 10 nm, including all 0.1 nm values and ranges therebetween.

The size and/or size distribution of inorganic nanotherapeutic imaging agent(s) and/or inorganic nanoimaging agent(s) can be determined by methods known in the art. In various examples, the size (which may be an average size) of an inorganic nanotherapeutic imaging agent or agents or an inorganic nanoimaging agent or agents is determined by dynamic light scattering, transmission electron spectroscopy, or the like, or a combination thereof.

In various examples, a composition further comprises one or more additional component(s). Non-limiting examples of materials which can be used as additional component(s) in a composition include sugars, such as, for example, lactose, glucose, sucrose, and the like: starches, such as, for example, corn starch, potato starch, and the like; cellulose, and its derivatives, such as, for example, sodium carboxymethyl cellulose, ethyl cellulose, cellulose acetate, and the like; powdered tragacanth: malt: gelatin: talc: excipients, such as, for example, cocoa butter, suppository waxes, and the like; oils, such as, for example, peanut oil, cottonseed oil, safflower oil, sesame oil, olive oil, corn oil, soybean oil, and the like; glycols, such as, for example, propylene glycol and the like; polyols, such as, for example, glycerin, sorbitol, mannitol, polyethylene glycol, and the like; esters, such as, for example, ethyl oleate, ethyl laurate, and the like: agar: buffering agents, such as, for example, magnesium hydroxide, aluminum hydroxide, and the like; alginic acid; pyrogen-free water; isotonic saline; Ringer's solution; ethyl alcohol; phosphate buffer solutions; and other non-toxic compatible substances employed in pharmaceutical formulations, and the like, and any combination thereof. (See, e.g., REMINGTON'S PHARM. SCI., 15th Ed. (Mack Publ. Co., Easton (1975)). In various examples, an additional component is a pharmaceutically acceptable carrier. Non-limiting examples of pharmaceutically acceptable carriers are found in: Remington: The Science and Practice of Pharmacy (2012) 22nd Edition, Philadelphia, PA. Lippincott Williams & Wilkins.

In an aspect, the present disclosure provides kits. In various examples, a kit comprises one or more inorganic nanotherapeutic agent(s), one or more inorganic nanoimaging agent(s) of the present disclosure or a combination thereof and/or one or more composition(s) of the present disclosure. Non-limiting examples of kits described herein.

In various examples, a kit comprises one or more inorganic nanotherapeutic agent(s) and/or one or more inorganic nanoimaging agent(s) and/or one or more composition(s) comprising the inorganic nanotherapeutic agent(s) and/or one inorganic nanoimaging agent(s), and instructions for use of the inorganic nanotherapeutic agent(s) and/or one the inorganic nanoimaging agent(s) and/or the composition(s) for carrying out (or in) a method of the present disclosure. In various examples, a kit comprises a closed or sealed package containing inorganic nanotherapeutic agent(s), inorganic nanoimaging agent(s) or a combination thereof and/or composition(s). In various examples, the package comprises one or more closed or sealed vials, bottles, blister (bubble) packs, or any other suitable packaging for the sale, distribution, or use of the inorganic nanotherapeutic agent(s), inorganic nanoimaging agent(s) or a combination thereof and/or composition(s). The printed material may include printed information. The printed information may be provided on a label, on a paper insert, printed on a packaging material, or the like. The printed information may include information that identifies the inorganic nanotherapeutic agent(s), inorganic nanoimaging agent(s) or a combination thereof and/or composition(s) in the package, the amounts and types of other active and/or inactive ingredients in the composition(s), and instructions for administration and/or use of the inorganic nanotherapeutic agent(s), inorganic nanoimaging agent(s) or a combination thereof and/or composition(s). The instructions may include information, such as, for example, the number of doses to take over a given period of time, and/or information directed to a pharmacist and/or another health care provider, such as, for example, a physician or the like, or a patient. The printed material may include an indication or indications that the inorganic nanotherapeutic agent(s), inorganic nanoimaging agent(s) or a combination thereof and/or composition(s) and/or any other agent provided therein is for treatment of a subject. In various examples, the kit includes a label describing the contents of the kit and providing indications and/or instructions regarding use of the contents of the kit to treat a subject.

The following Statements describe various examples of inorganic nanotherapeutic imaging agents, inorganic nanoimaging agents, compositions, and methods of the present disclosure that are not intended to be limiting in any manner:

Statement A1. An inorganic nanoimaging agent (e.g., an engineered nanoparticle or the like) comprising one or more dye group(s), one or more sulfur atom group(s), one or more radioisotope group(s), one or more heavy atom group(s), or the like, or any combination thereof, or any combination thereof, where the dye group(s), the sulfur atom group(s), the radioisotope group(s), the heavy atom group(s), targeting group(s), or the like, if present, are independently disposed on a surface of an inorganic nanoimaging agent (e.g., covalently or non-covalently bonded to a surface of an inorganic nanoimaging agent or the like) covalently or non-covalently bonded (e.g., chelated or the like) to the matrix (e.g., silica matrix, aluminosilicate matrix, or the like) of an inorganic nanoimaging agent (e.g., as described herein or the like).
Statement A2. An inorganic nanoimaging agent (e.g., an engineered nanoparticle or the like) (e.g., for imaging, diagnosing, and/or treating joints(s) (or any portion thereof) (such as, for example, one or more tissue(s) associated with the joints(s) (e.g., cartilage, synovial membrane, ligaments, tendons, bursas, synovial fluid, and/or meniscus or the like) and/or cells (and/or cell structures) associated with tissues such as cartilage, synovial membrane, ligaments, tendons, bursas, synovial fluid, and/or meniscus), one or more musculoskeletal tissue(s) (or any portion thereof), or any combination thereof in a sample or a subject, the inorganic nanoimaging agent (e.g., the engineered nanoparticle or the like) comprising a silica-poly(ethylene glycol)(PEG) core particle conjugated to one or more of: a fluorescent moiety (e.g., for imaging joints(s) (or any portion thereof) (such as, for example, one or more tissue(s) associated with the joints(s) (e.g., cartilage, synovial membrane, ligaments, tendons, bursas, synovial fluid, and/or meniscus or the like) and/or cells (and/or cell structures) associated with tissues such as cartilage, synovial membrane, ligaments, tendons, bursas, synovial fluid, and/or meniscus), one or more musculoskeletal tissue(s) (or any portion thereof), or any combination thereof); a targeting moiety (e.g., for targeting to joints(s) (or any portion thereof) (such as, for example, one or more tissue(s) associated with the joints(s) (e.g., cartilage, synovial membrane, ligaments, tendons, bursas, synovial fluid, and/or meniscus or the like) and/or cells (and/or cell structures) associated with tissues such as cartilage, synovial membrane, ligaments, tendons, bursas, synovial fluid, and/or meniscus), one or more musculoskeletal tissue(s) (or any portion thereof), or any combination thereof); and/or a therapeutic moiety (e.g., for treating joints(s) (or any portion thereof) (such as, for example, one or more tissue(s) associated with the joints(s) (e.g., cartilage, synovial membrane, ligaments, tendons, bursas, synovial fluid, and/or meniscus or the like) and/or cells (and/or cell structures) associated with tissues such as cartilage, synovial membrane, ligaments, tendons, bursas, synovial fluid, and/or meniscus), one or more musculoskeletal tissue(s) (or any portion thereof), or any combination thereof).
Statement A3. An inorganic nanoimaging agent (e.g., an engineered nanoparticle or the like) according to any one of the preceding Statements, where the engineered nanoparticle(s) comprise a longest linear dimension (such as, for example, a diameter or the like) of about 2 nanometers (nm) to about 1 micrometer (micron), including all 0.1 nm values and ranges therebetween (about 2 nm to about 10 nm).
Statement B1. A composition (e.g., for imaging, diagnosing, and/or treating joints(s) (or any portion thereof) (such as, for example, one or more tissue(s) associated with the joints(s) (e.g., cartilage, synovial membrane, ligaments, tendons, bursas, synovial fluid, and/or meniscus or the like) and/or cells (and/or cell structures) associated with tissues such as cartilage, synovial membrane, ligaments, tendons, bursas, synovial fluid, and/or meniscus), one or more musculoskeletal tissue(s) (or any portion thereof), or any combination thereof in a sample or a subject: a plurality of one or more inorganic nanoimaging agent (e.g., an engineered nanoparticle or the like) as described in Statement A1 and/or Statement A2.
Statement C1. A method for (e.g., for imaging, diagnosing, and/or treating joints(s) (or any portion thereof) (such as, for example, one or more tissue(s) associated with the joints(s) (e.g., cartilage, synovial membrane, ligaments, tendons, bursas, synovial fluid, and/or meniscus or the like) and/or cells (and/or cell structures) associated with tissues such as cartilage, synovial membrane, ligaments, tendons, bursas, synovial fluid, and/or meniscus), one or more musculoskeletal tissue(s) (or any portion thereof), or any combination thereof in a sample or a subject: contacting the sample or the subject with a composition of Statement B1 (or a composition otherwise described herein).
Statement D1. A method for (e.g., for imaging, diagnosing, and/or treating joints(s) (or any portion thereof) (such as, for example, one or more tissue(s) associated with the joints(s) (e.g., cartilage, synovial membrane, ligaments, tendons, bursas, synovial fluid, and/or meniscus or the like) and/or cells (and/or cell structures) associated with tissues such as cartilage, synovial membrane, ligaments, tendons, bursas, synovial fluid, and/or meniscus), one or more musculoskeletal tissue(s) (or any portion thereof), or any combination thereof in a sample or a subject: contacting the sample or the subject with a composition of Statement B1 (or a composition otherwise described herein); directing excitation electromagnetic radiation into the sample or the subject, thereby exciting at least one of the one or more dye molecule(s), if present, one or more radioisotope(s), if present, or one or more iodide(s), if present, or the combination thereof resulting in emission (or excitation and emission) of electromagnetic radiation from the dye molecule(s), if present, the radioisotope(s), if present, or the iodide(s), if present, or the combination thereof; detecting at least a portion of the emitted electromagnetic radiation, the detected electromagnetic radiation having been emitted by the dye molecule(s), if present, the radioisotope(s), if present, or the iodide(s), if present, or the combination thereof; and processing signals corresponding to the detected electromagnetic radiation to provide one or more image(s), diagnoses, and/or to monitor treatment of joint(s) (or the portion thereof), the musculoskeletal tissue(s) (or the portion thereof), or the combination thereof in the sample or the subject.
Statement 2. A method according to any one of the preceding Statements, where the electromagnetic radiation is directed into the sample or subject.
Statement 3. A method according to any of the preceding Statements, where the electromagnetic radiation is directed into a region, where the region is within the sample or subject.
Statement 4. A method according to any one of the preceding Statements, where the electromagnetic radiation comprises one or more wavelengths from about 400 to about 1700 nm, including all nm values and ranges therebetween (e.g., about 400 to about 1200 nm).
Statement 5. A method according to any one of the preceding Statements, where the irradiation is carried out using one or more laser(s) (e.g., a single laser or the like) or the like. A method according to any one of the preceding Statements, where the electromagnetic radiation is provided by tunable wavelength laser or the like.
Statement 6. A method according to any one of the preceding Statements, where the directing, detecting, and processing is fluorescence imaging, bioluminescence imaging, or the like, or any combination thereof.
Statement 7. A method according to any one of the preceding Statements, where at least a portion or all of the inorganic nanoimaging agent(s) (e.g., the engineered nanoparticle(s) or the like) is/are aluminosilicate nanoparticles or the like) (e.g., aluminosilicate nanoparticle(s) comprising at least one organic dye molecule covalently bonded to the aluminosilicate network of the individual aluminosilicate nanoparticles) and the imaging is super-resolution imaging (e.g., optical imaging or the like with resolution below Abbe's diffraction limit).
Statement 8. A method according to Statement 15, where the image(s) are fluorescence image(s) (which independently may be a super-resolution image) or a sequence of fluorescence image(s) which can be processed to obtain an optical super-resolution image (e.g., an optical image or the like with resolution below Abbe's diffraction limit) of the sample or portion thereof or the individual or a portion thereof.
Statement 9. A method according to any one of the preceding Statements, where the method is an optical super-resolution microcopy method including, but not limited to, ground state depletion (GSD) microscopy, stochastic optical reconstruction microscopy (STORM), direct stochastic optical reconstruction microscopy (dSTORM), stimulated emission and depletion (STED), or photoactivated localization microscopy (PALM), or the like, or any combination thereof.
Statement 10. A method according to any one of the preceding Statements, the method further comprising targeting, diagnosing, treating, preventing, or any combination thereof, a current or potential disease, disease state, condition, disorder, side effect, or any combination thereof, in a subject. In some cases, the sample is a biopsy sample or a resected tissue sample.
Statement 11. A method according to Statement 10, where the current or potential disease is cancer, and the method further comprises one or more chemotherapy treatment(s), one or more radiation treatment(s), one or more photodynamic therapy treatment(s), one or more surgical intervention(s) (e.g., surgical procedure(s), or the like), or the like, or any combination thereof.
Statement 12. A method of treating a subject diagnosed with or suspected of having or with increased risk due to injury of having an inflammatory joint disease (such as, for example, arthritis (e.g., rheumatoid arthritis and/or osteoarthritis, or the like, or both) or the like) comprising: administering one or more inorganic nanotherapeutic agent(s) to the subject, where one or more symptoms of the individual's rheumatoid arthritis and/or osteoarthritis is treated.
Statement 13. A method according to Statement 12, where the inorganic nanotherapeutic agent(s) independently comprise one or more targeting group(s) and one or more therapeutic group(s), where the one or more targeting group(s) and the one or more therapeutic group(s) are independently disposed on a surface of the inorganic nanotherapeutic agent.
Statement 14. A method according to Statement 12 or 13, where the targeting group(s) is/are independently for each inorganic nanotherapeutic agent chosen from tissue targeting groups, tissue-resident cell targeting groups, and the like, and any combination thereof.
Statement 15. A method according to any one of Statements 12 to 14, where the therapeutic group(s) is/are independently for each inorganic nanotherapeutic agent chosen from anti-inflammatory therapeutic groups, catabolic groups, anti-catabolic therapeutic groups, anabolic therapeutic groups, pro-anabolic groups, bioenergetic groups, viscosupplement groups, corticosteroid groups, steroid groups, antioxidant groups, gene therapy groups, nucleotide groups (such as, for example, RNA groups (e.g., mRNA groups, siRNA groups, and the like), and the like) and any combination thereof.
Statement 16. A method according to any one of Statements 12 to 15, where the inorganic nanotherapeutic agent(s) independently further comprise one or more fluorescent group(s), one or more sulfur atom group(s), one or more radioisotope group(s), one or more heavy atom group(s), or the like, or any combination thereof, where the fluorescent group(s), the sulfur atom group(s), the radioisotope group(s), or the heavy atom group(s), or the like are independently disposed on a surface of the inorganic nanoimaging agent.
Statement 17. A method according to any one of Statements 12 to 16, where the inorganic nanotherapeutic agent(s) independently comprise(s) a silica matrix, an aluminosilicate matrix, or the like.
Statement 18. A method according to any one of Statements 12 to 17, where inorganic nanotherapeutic agent(s) comprise a silica-poly(ethylene glycol)(PEG) core-shell particle, or the like.
Statement 19. A method according to any one of Statements 12 to 18, where the inorganic nanotherapeutic agent(s) are independently chosen from nanoparticles, nanorings, and nanocages, and the like, and any combination thereof.
Statement 20. A method according to any one of Statements 12 to 19, where at least a portion or all the inorganic nanoimaging agent(s) target(s) chondrocytes, tendon cells, ligament cells, meniscus cells, synovial cells, bone marrow cells, blood cells, lymphocytes, macrophages, fibroblasts, bone-lining cells, osteoblasts, osteoclasts, osteocytes, nerve cells, blood vessel cells, or the like, or any combination thereof, or a portion thereof.
Statement 21. A method according to any one of Statements 12 to 20, where at least a portion of the inorganic nanotherapeutic agent(s) reside(s) in one or more joints(s) or any portion thereof, one or more musculoskeletal tissue(s) or any portion thereof, or any combination thereof in the subject for at least 14 days, at least 21 days, at least 28 days, or at least 3 months.
Statement 22. A method according to any one of Statements 12 to 21, where the joint(s) are chosen from ankle joints, knee joints, hipjoints, finger joints, wrists, elbows, temporomandibular, facet, and shoulder joints, and the like, and any combination thereof and/or the musculoskeletal tissue(s) are chosen from cartilage, synovial membrane, ligaments, tendons, tendon sheaths, bursas, bone, synovial fluid, meniscus, and meniscal enthesis, and the like, and any combination thereof.
Statement 23. A method according to any one of Statements 12 to 22, where the musculoskeletal tissue(s) independently comprise cells and/or cell structures associated with cartilage, synovial membrane, ligaments, tendons, tendon sheaths, bursas, bone, synovial fluid, or meniscus, or the like, or any combination thereof and at least a portion of the inorganic nanotherapeutic agent(s) reside(s) in the cells and/or the cell structures.
Statement 24. A method according to any one of Statements 12 to 23, where at least a portion of the inorganic nanotherapeutic agent(s) are taken up by in the chondrocytes, meniscus cells, ligament cells, tendon cells, lymphocytes, bone cells, or synovial cells, or the like, or any combination thereof of the individual.
Statement 25. A method according to any one of Statements 12 to 24, where the inorganic nanotherapeutic agent(s) are present in composition and the composition is administered to the subject.
Statement 26. A method according to any one of Statements 12 to 25, where the inorganic nanoimaging agent(s) are present in a composition at concentration of about 1 nanomolar to about 100 micromolar.
Statement 27. A method according to any one of Statements 12 to 26, where the inorganic nanoimaging agent(s) is/are administered proximate to the joint(s) or any portion thereof, the musculoskeletal tissue(s), or any combination thereof of the subject.
Statement 28. A method according to any one of Statements 12 to 27, where the administration is subcutaneous administration, intravenous administration, intraarticular administration, infrapatellar fat pad administration, or intramarrow administration, or the like, or any combination thereof.
Statement 29. A method according to any one of Statements 12 to 28, where the administration comprises a single dose or multiple doses of inorganic nanotherapeutic agent(s).
Statement 30. A method according to any one of Statements 12 to 29, where the administration is intraarticular administration and about 10 microliters to about 10 milliliters is administered.
Statement 31. A method according to any one of Statements 12 to 30, where the inorganic nanotherapeutic agent(s) comprise a longest linear dimension of about 2 nanometers (nm) to about 1 micrometer and, optionally, the administration is intraarticular.
Statement 32. A method according to any one of Statements 12 to 31, where the inorganic nanoimaging agent(s) comprise a longest linear dimension of about 2 nanometers (nm) to about 10 nm, and, optionally, the administration is subcutaneous.
Statement 33. A method according to any one of Statements 12 to 32, where the inorganic nanotherapeutic agent(s) do not comprise one or more fluorescent group(s), one or more sulfur atom group(s), one or more radioisotope group(s), one or more heavy atom group(s), or the like, or any combination thereof.
Statement 34. A method according to any one of Statements 12 to 33, where the method does not comprise administration of any core-shell nanoparticles (other than inorganic nanotherapeutic agent(s) and/or inorganic nanoimaging agent(s)) comprising one or more fluorescent group(s), one or more sulfur atom group(s), one or more radioisotope group(s), one or more heavy atom group(s), or the like, or any combination thereof.
Statement 35. A method according to any one of Statements 12 to 34, where at least a portion of the inorganic therapeutic agents comprise one or more fluorescent group(s), one or more sulfur atom group(s), one or more radioisotope group(s), one or more heavy atom group(s), or the like, or any combination thereof, the method further comprises imaging one or more joints(s) or any portion thereof, one or more musculoskeletal tissue(s), or the like, or any portion thereof, or any combination thereof the individual.
Statement 36. A method according to any one of Statements 12 to 35, where the fluorescent group(s) is/are independently chosen from fluorescent organic dye group(s), or fluorescent protein group(s), fluorescent peptide groups, fluorescent small molecule groups, and the like, and any combination thereof.
Statement 37. A method according to Statements 35 or 36, where the musculoskeletal tissue(s) are chosen from cartilage, synovial membrane, ligaments, tendons, tendon sheaths, bursas, bone, synovial fluid, and meniscus, and the like, and any combination thereof.
Statement 38. A method according to any one of Statements 35 to 37, where the musculoskeletal tissue(s) independently comprise cells and/or cell structures associated with cartilage, synovial membrane, ligaments, tendons, tendon sheaths, bursas, bone, synovial fluid, or meniscus, or the like, or any combination thereof.
Statement 39. A method according to any one of Statements 35 to 38, where the imaging comprises: directing excitation electromagnetic radiation into the sample or the subject, thereby exciting at least one of the one or more fluorescent groups, if present, or one or more radioisotope(s), if present, or a combination thereof resulting in emission or excitation and emission of electromagnetic radiation from the fluorescent group(s), if present, or the radioisotope(s), if present, or the combination thereof: detecting at least a portion of the emitted electromagnetic radiation, the detected electromagnetic radiation having been emitted by the fluorescent group(s), if present, or the radioisotope(s), if present, or the combination thereof; and processing signals corresponding to the detected electromagnetic radiation to provide one or more image(s) of the joint(s) or the portion thereof, the musculoskeletal tissue(s), or the like, or the portion thereof, or the combination thereof in the sample or the subject.
Statement 40. A method according to Statement 39, where the electromagnetic radiation comprises one or more wavelength(s) from about 400 to about 1700 nm.
Statement 41. A method according to Statement 39 or 40, where the image(s) is/are fluorescence images.
Statement 42. A method according to any one of Statements 35 to 41, where the electromagnetic radiation is directed into a region, where the region is within the sample or the subject and the region comprises the one or more joint(s) or the portion thereof, one or more musculoskeletal tissue(s), or the like, or the portion thereof, or the combination thereof.
Statement 43. A method according to any one of Statements 35 to 42, where the imaging comprises multiphoton imaging.
Statement 44. A method according to any one of Statements 35 to 43, where the imaging is super-resolution imaging.
Statement 45. A method according to any one of Statements 35 to 44, where at least a portion or all the inorganic nanoimaging agent(s) is/are aluminosilicate nanoparticle(s), the aluminosilicate nanoparticle(s) independently comprising at least one organic dye molecule covalently bonded to the aluminosilicate network of the individual aluminosilicate nanoparticles and the imaging is optical super-resolution imaging.
Statement 46. A method according to any one of Statements 35 to 45, where the image(s) resulting from the imaging are fluorescence image(s) or a sequence of fluorescence image(s) which can be processed to obtain an optical super-resolution image of the sample or portion thereof or the individual or a portion thereof.
Statement 47. A method according to any one of Statements 35 to 46, where the imaging is an optical super-resolution microcopy method.
Statement 48. A method according to Statement 47, where the optical super-resolution microcopy method is ground state depletion (GSD) microscopy, stochastic optical reconstruction microscopy (STORM), direct stochastic optical reconstruction microscopy (dSTORM), stimulated emission and depletion (STED), or photoactivated localization microscopy (PALM), or the like, or any combination thereof.
Statement 49. A method according to any one of Statements 35 to 48, further comprising an incubation time prior to the imaging.
Statement 50. A method according to any one of Statements 12 to 49, further comprising an intravital fluorescence imaging method.
Statement 51. A method according to any one of Statements 35 to 50, further comprising determining the treatment of the individual using the imaging.
Statement 52. A method according to any one of Statements 35 to 51, further comprising diagnosing the individual using the imaging.
Statement 53. An inorganic nanotherapeutic agent comprising one or more targeting group(s) is/are chosen Type II collagen targeting peptides (e.g., WYRGL (SEQ ID NO. 1) or the like) or the like), or
Statement 54. An inorganic nanotherapeutic agent according to Statement 53, where the targeting group(s) is/are independently for each inorganic nanotherapeutic agent chosen from IL-1 Ra (receptor agonist), TNF-a (alpha), or the like, or any combination thereof (which may independently be disposed on a surface of the inorganic nanotherapeutic agent or a PEG group comprises the therapeutic group), and any combination thereof.
Statement 55. An inorganic nanotherapeutic agent according to Statement 53 or 54, where the therapeutic group(s) is/are independently for each inorganic nanotherapeutic agent chosen IL-1 Ra (receptor agonist), TNF-a (alpha), or the like, or any combination thereof (which may independently be disposed on a surface of the inorganic nanotherapeutic agent or a PEG group comprises the therapeutic group), and any combination thereof.
Statement 56. An inorganic nanotherapeutic agent according to any one of Statements 53 to 55, where the inorganic nanotherapeutic agent(s) independently further comprise one or more fluorescent group(s), one or more sulfur atom group(s), one or more radioisotope group(s), one or more heavy atom group(s), or any combination thereof, where the fluorescent group(s), the sulfur atom group(s), the radioisotope group(s), the heavy atom group(s), or the targeting group(s), if present, are independently disposed on a surface of the inorganic nanoimaging agent.
Statement 57. An inorganic nanotherapeutic agent according to any one of Statements 53 to 56, where the fluorescent group(s) is/are independently chosen from organic dye group(s) (such as, for example, Cyanine-5 (Cy5) groups and the like) and any combination thereof.
Statement 58. An inorganic nanotherapeutic agent according to any one of Statements 53 to 57, where the radioisotope groups(s) is/are independently chosen from ¹²⁴I, ⁸⁹Zr, ¹¹C, ¹⁵O, ¹⁸F, ⁶⁴Cu, or ⁶⁸Ga, and the like, and any combination thereof.
Statement 59. An inorganic nanotherapeutic agent according to any one of Statements 53 to 58, where the heavy atom(s) is/are independently chosen from iodine atom, bromine atom. Au ions, Ag ions, Pb ions, Ti ions, Bi ions, Pt ions, In ions, Sn ions, Sb ions or Pd ions, and the like, and structural derivatives thereof, and any group derived therefrom, or any combination thereof.
Statement 60. An inorganic nanotherapeutic agent according to any one of Statements 53 to 59, where the inorganic nanotherapeutic agent comprises a silica matrix, an aluminosilicate matrix, or the like.
Statement 61. An inorganic nanotherapeutic agent according to any one of Statements 53 to 60, where inorganic nanotherapeutic agent is a silica-poly(ethylene glycol)(PEG) core particle, or the like.
Statement 62. An inorganic nanotherapeutic agent according to any one of Statements 53 to 61, where the inorganic nanotherapeutic agent(s) are independently chosen from nanoparticles, nanorings, and nanocages, and the like.
Statement 63. An inorganic nanotherapeutic agent according to any one of Statements 53 to 62, where inorganic nanotherapeutic agent is configured for use in a method of claim 1.
Statement 64. An inorganic nanotherapeutic agent according to any one of Statements 53 to 63, where the inorganic nanotherapeutic agent comprises a longest linear dimension of about 2 nanometers (nm) to about 1 micrometer (micron).
Statement 65. An inorganic nanotherapeutic agent according to any one of Statements 53 to 65, where the inorganic nanotherapeutic agent comprises a longest linear dimension of about 2 nm to about 10 nm.
Statement 66. A composition comprising one or more inorganic nanotherapeutic agent(s) independently according to any one of Statements 53 to 65.
Statement 67. A composition according to Statement 66, where the composition comprises inorganic nanotherapeutic agent(s) at a concentration of 10 nanomolar to 100 micromolar.
Statement 68. A composition according to Statement 66 or 67, where the composition is suitable for subcutaneous administration, intravenous administration, intraarticular administration, infrapatellar fat pad administration, or intramarrow administration.
Statement 69. A composition according to any one of Statements 66 to 68, where the composition is suitable for intraarticular administration.
Statement 70. A composition according to any one of Statements 66 to 69, where the inorganic nanoimaging agent(s) comprise a longest linear dimension of about 2 nanometers (nm) to about 100 nanometers (nm) and, optionally, the administration is intravenous.
Statement 71. A composition according to any one of Statements 66 to 70, where the inorganic nanoimaging agent(s) comprise a longest linear dimension of about 2 nanometers (nm) to about 10 nm, and, optionally, the administration is subcutaneous.
Statement 72. A method for imaging one or more joints(s) or any portion thereof, one or more musculoskeletal tissue(s), or the like, or any portion thereof, or any combination thereof in a sample or a subject: contacting the sample or the subject with one or more inorganic nanoimaging agent(s); and imaging the sample or the subject.
Statement 73. A method according to Statement 72, where the musculoskeletal tissue(s) are chosen from cartilage, synovial membrane, ligaments, tendons, bursas, synovial fluid, and meniscus, and the like, and any combination thereof.
Statement 74. A method according to Statement 72 or 73, where the musculoskeletal tissue(s) independently comprise cells and/or cell structures associated with cartilage, synovial membrane, ligaments, tendons, bursas, synovial fluid, or meniscus, or the like, or any combination thereof.
Statement 75. A method according to any one of Statements 72 to 74, where the inorganic nanoimaging agent(s) independently comprise one or more fluorescent group(s), one or more sulfur atom group(s), one or more radioisotope group(s), one or more heavy atom group(s), therapeutic group(s), or the like, or any combination thereof, where the fluorescent group(s), the sulfur atom group(s), the radioisotope group(s), the heavy atom group(s), targeting group(s), or therapeutic group(s), if present, or the like, if present are independently disposed on a surface of the inorganic nanoimaging agent.
Statement 76. A method according to any one of Statements 72 to 75, where the inorganic nanoimaging agent(s) independently comprise: fluorescent group(s); and targeting group(s); and, optionally, therapeutic group(s).
Statement 77. A method according to any one of Statements 72 to 76, where at least a portion or all the inorganic nanoimaging agent(s) target(s) osteocytes or a portion thereof, bone marrow cells, blood cells, lymphocytes, fibroblasts, bone-lining cells, osteoblasts, osteoclasts, chondrocytes, nerve cells, or blood vessel cells, or the like, or any combination thereof, or a portion thereof.
Statement 78. A method according to any one of Statements 72 to 77, where the inorganic nanoimaging agent(s) is/are administered proximate to the joint(s) or any portion thereof, the musculoskeletal tissue(s) or any portion thereof, or the like, or any combination thereof of a subject.
Statement 79. A method according to any one of Statements 72 to 78, where the one or more joints(s) or any portion thereof, the one or more musculoskeletal tissue(s) or any portion thereof), or any combination thereof is manipulated prior to and/or during the imaging.
Statement 80. A method according to any one of Statements 72 to 79, where the imaging comprises: directing excitation electromagnetic radiation into the sample or the subject, thereby exciting at least one of the one or more fluorescent groups, if present or one or more radioisotope(s), if present, or a combination thereof resulting in emission or excitation and emission of electromagnetic radiation from the fluorescent group(s), if present, or the radioisotope(s), if present, or the combination thereof; detecting at least a portion of the emitted electromagnetic radiation, the detected electromagnetic radiation having been emitted by the fluorescent group(s), if present, or the radioisotope(s), if present, or the combination thereof; and processing signals corresponding to the detected electromagnetic radiation to provide one or more image(s) of the joint(s) or the portion thereof, the musculoskeletal tissue(s) or the portion thereof, or the combination thereof in the sample or the subject.
Statement 81. A method according to any one of Statements 72 to 80, where the image(s) is/are fluorescence images.
Statement 82. A method according to any one of Statements 72 to 81, where the electromagnetic radiation is directed into a region, where the region is within the sample or the subject and the region comprises the one or more joint(s) or the portion thereof, one or more musculoskeletal tissue(s) or the portion thereof, or the combination thereof.
Statement 83. A method according to any one of Statements 72 to 82, where the imaging comprises multiphoton imaging.
Statement 84. A method according to any one of Statements 72 to 84, where the imaging is super-resolution imaging.
Statement 85. A method according to any one of Statements 72 to 79, where at least a portion or all the inorganic nanoimaging agent(s) is/are aluminosilicate nanoparticle(s), the aluminosilicate nanoparticle(s) independently comprising at least one organic dye group covalently bonded (e.g., an organic dye molecule covalently bonded) to the aluminosilicate network of the individual aluminosilicate nanoparticles and the imaging is super-resolution imaging.
Statement 86. A method according to any one of Statements 72 to 85, where the imaging is an optical super-resolution microcopy method.
Statement 87. A method according to any one of Statements 72 to 86, where the optical super-resolution microcopy method is ground state depletion (GSD) microscopy, stochastic optical reconstruction microscopy (STORM), direct stochastic optical reconstruction microscopy (dSTORM), stimulated emission and depletion (STED), or photoactivated localization microscopy (PALM), or the like, or any combination thereof.
Statement 88. A method according to any one of Statements 72 to 87, further comprising diagnosing the diagnosing the joints(s) or any portion thereof, one or more musculoskeletal tissue(s) or any portion thereof, or the combination thereof using the images of the sample or the subject; and/or where at least a portion or all the inorganic nanoimaging agent(s) comprise one or more therapeutic agent(s) and the one or more joints(s) or any portion thereof, the one or more musculoskeletal tissue(s) or any portion thereof are treated.
Statement 89. A method according to any one of Statements 72 to 88, further comprising targeting, diagnosing, treating, preventing, or any combination thereof, a current or potential disease, disease state, condition, disorder, side effect, or the like, or any combination thereof, in a subject.
Statement 90. A method according to any one of Statements 72 to 89, where the current or potential disease or disease state is an inflammatory joint disease (such as, for example, arthritis (e.g., rheumatoid arthritis and osteoarthritis, and the like, and both) and the like), a cancer or cancers, or the like, or the conditions, the disorders, or the side effects, or the like are related to same.

The steps of the methods described in the various examples disclosed herein are sufficient to carry out a method of the present disclosure. Thus, in various examples, a method consists essentially of a combination of the steps of the methods disclosed herein. In various other examples, a method consists of such steps.

The following examples are presented to illustrate the present disclosure. They are not intended to be limiting in any manner.

### Example 1

This Example describes, inter alia, inorganic imaging agents, compositions, and methods of the present disclosure.

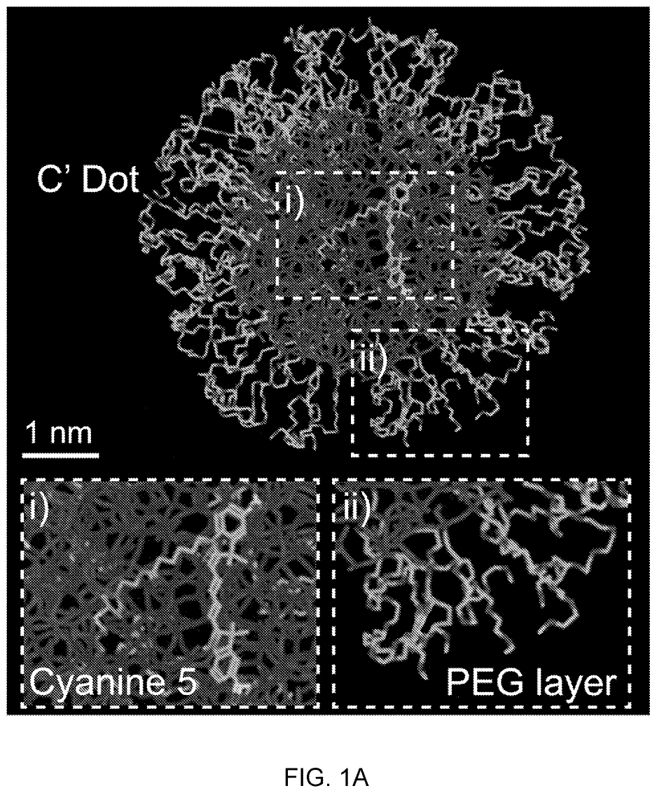

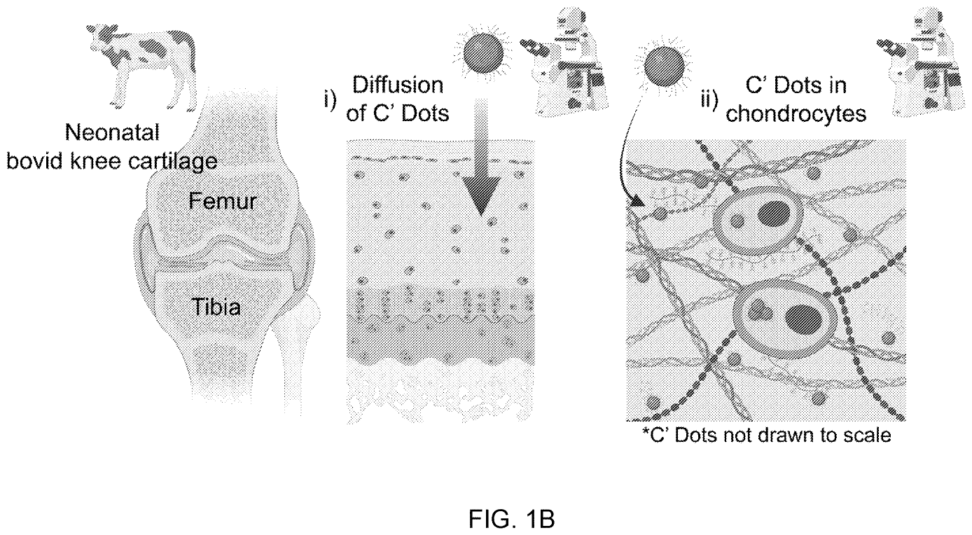

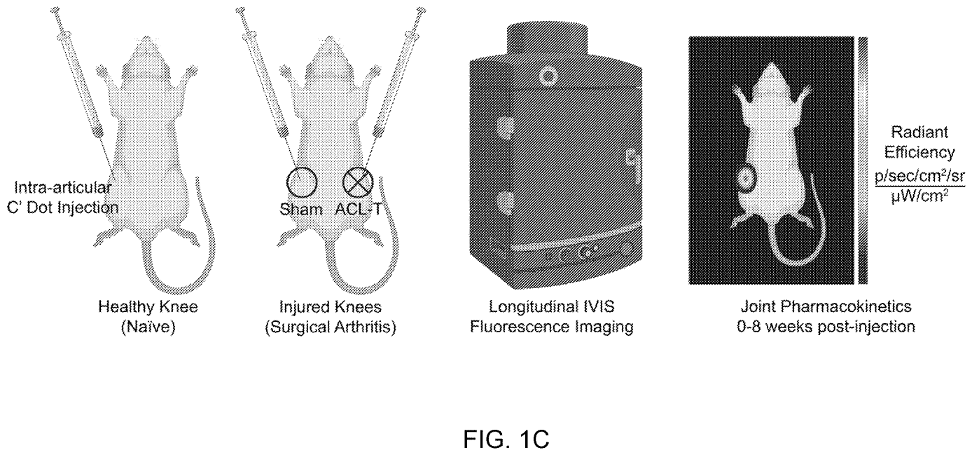

C′ Dots were investigated as a therapeutic delivery platform for OA (FIG. 1). The aims of this study were to 1) understand diffusion kinetics of C′ Dots in cartilage (FIG. 1B (i)), 2) probe intra-tissue and intra-cellular processing dynamics and retention kinetics in whole tissue explants (FIG. 1B (ii)), and 3) characterize in vivo joint clearance PK of C′ Dots in naïve and surgically injured rat knees (FIG. 1C).

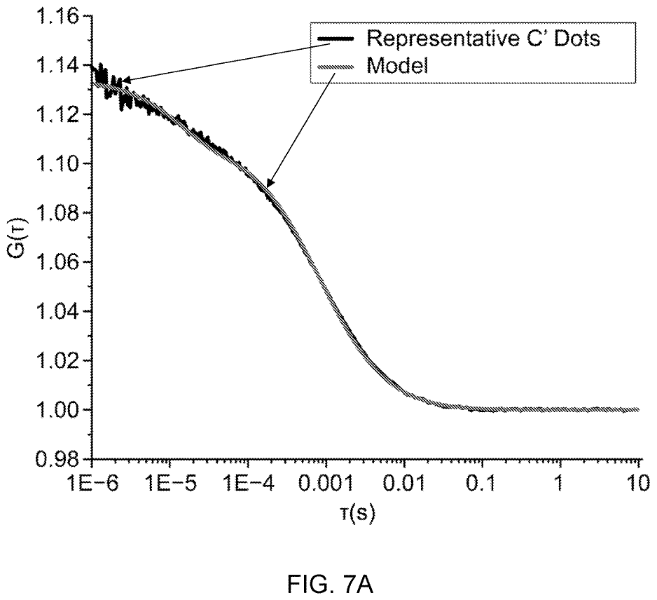

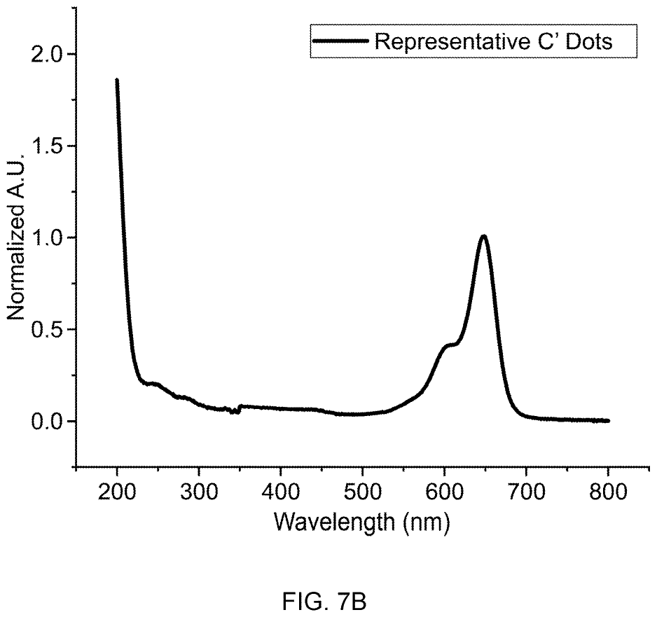

Materials and Methods. C′ Dot Synthesis. Molecularly engineered fluorescent silica core-poly(ethylene glycol) (PEG) shell hybrid conjugates (Cornell Prime Dots, or C, Dots) were synthesized under sterile conditions and purified as described previously. Briefly, C′ Dots are prepared in a “one-pot” synthesis in water where the final step of poly(ethylene glycol) surface conjugation (PEGylation) stops the addition of silica shells and stabilizes the C′ Dots at a 6 nm diameter. Synthesized C′ Dots were filtered via gel permeation chromatography (representative batch. FIG. 7B) and analyzed using fluorescence correlation spectroscopy (FIG. 7A). All C′ Dot formulations were stored at room temperature and sterile-filtered just before use in subsequent explant culture or animal experiments. Sulfo-Cyanine-5-carboxylic acid (Cy5) free fluorophore was obtained from Lumiprobe for preparation of the Cy5 injectate.

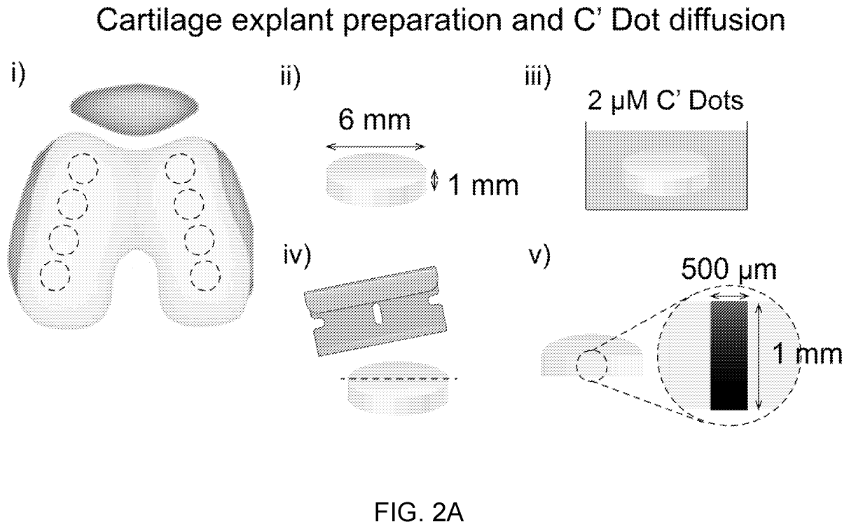

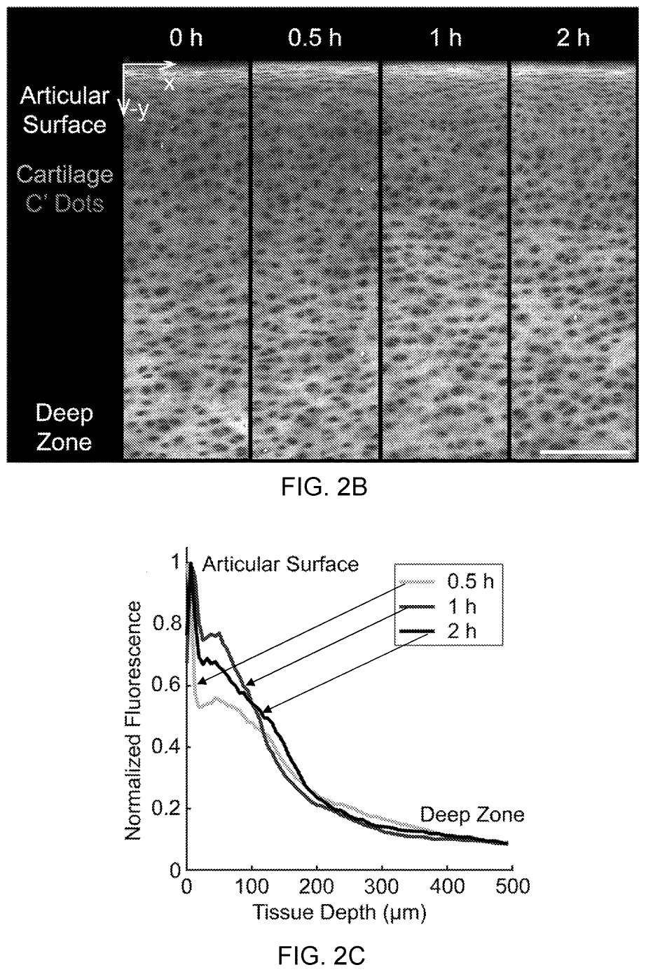

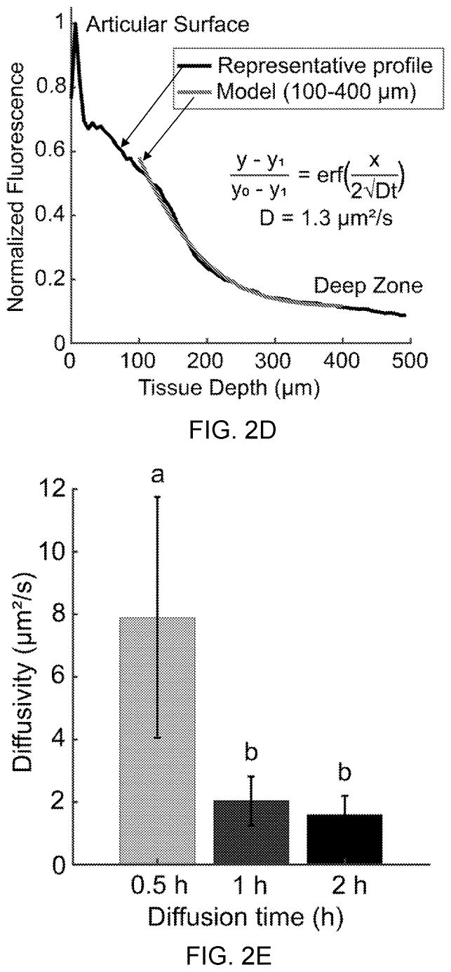

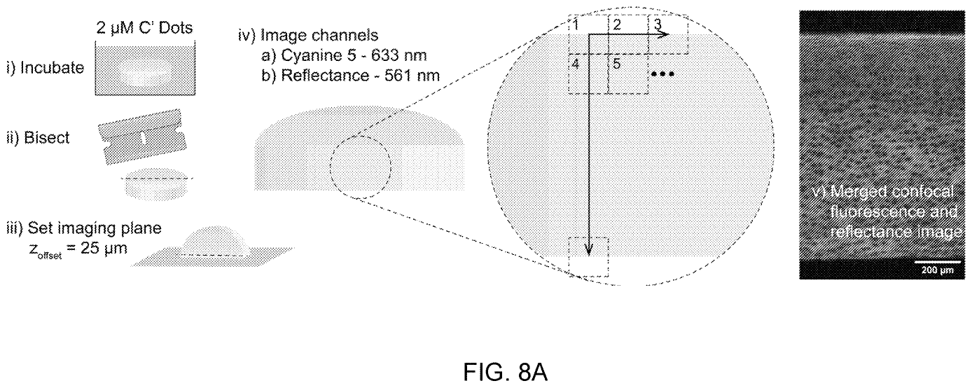

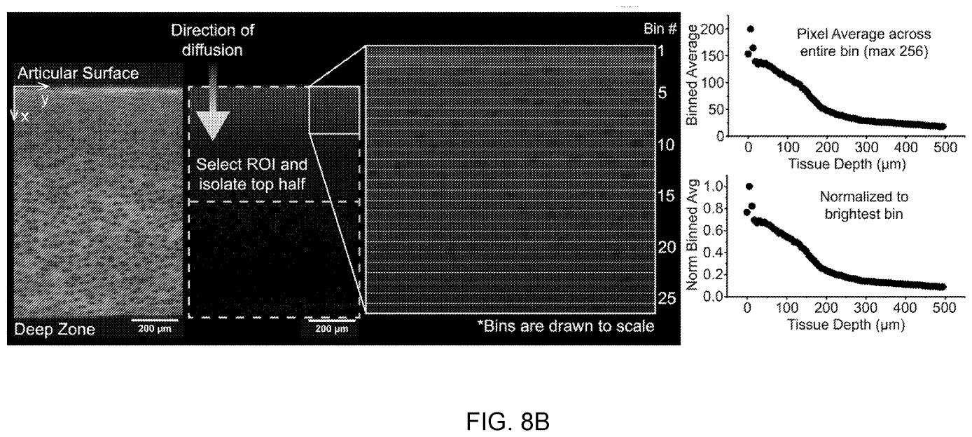

Diffusion Studies, Imaging, and Analysis. Sample preparation: Cylindrical cartilage plugs (6 mm diameter and 1 mm deep, n=5-6 per condition) were cut from the patellofemoral groove of 1-3 day old bovine stifle joints and randomly assigned to C′ Dot groups. C′ Dots in phosphate-buffered saline passively (2 μM) diffused through the articular surface of submerged cartilage similarly to previously established methods (FIG. 2A). Imaging: Confocal imaging assessed C′ Dot penetration from the cut plane in bisected samples (FIG. 2A-B, FIG. 8A). C′ Dot fluorescence and cartilage reflectance were recorded utilizing the 633 nm and 561 nm laser lines, respectively. Inage Analysis: The experiment was modeled as a 1D diffusion problem under the assumption that C′ Dots only penetrated from the articular surface in the imaged region. A depth-dependent profile of C′ Dot fluorescence was created by averaging pixel values in 6 μm bins parallel to the articular surface, using a custom MATLAB code (FIG. 8B). This profile was normalized prior to calculating effective diffusivity. These data were fitted to a 1D diffusion equation derived from Fick's 2^{nd} law to obtain effective diffusivity, D_{eff} in equation (1).

$$\frac{y - {y}_{1}}{{y}_{0} - {y}_{1}} = erf ( \frac{x}{2 \sqrt{{D}_{eff} t}} ) \qquad ( 1 )$$

In this 1D diffusion model, y₁ represents the initial concentration (x=0) in the normalized fluorescence profile (FIGS. 8 A and B) along x (units μm), while y₀ is the end concentration (x=500). Diffusivity was modeled from 100-400 μm in depth from the articular surface where the variation in local diffusivity is lowest. The parameters y₀, y₁, and D_{eff} were fit using Origin Pro software for all incubation times (R²>0.98, RMSE<0.05).

Chondrocyte Internalization Studies, Imaging, and Analysis. Sample preparation. Cylindrical cartilage plugs (6 mm in diameter and 2 mm deep, n=3 per group) were cut from the patellofemoral groove of 1-3 day old bovine stifle joints and randomly assigned to C′ Dot or control groups. C′ Dots (2 μM) in supplemented media (DMEM with 4.5 g/L glucose and L-glutamine, 10% fetal bovine serum, and 1% AbAm) passively diffused through the articular surface and radial edges of submerged cartilage plugs. After 24 hours, C′ Dot plugs were moved to fresh media that was changed once per day until imaging. Control plugs remained in C′ Dot-free media for the same incubation time. Imaging. Cartilage plugs were bisected with a lubricated blade (bovine synovial fluid) for fluorescence staining (30 minutes Calcein, AM & Sytox Blue nucleic acid stain, 30 minutes lysosome stain, or 24 hours endosome transfection). Tissue penetration and cellular internalization of C′ Dots were assessed at the bisected plane using a Zeiss 710 inverted confocal microscope and the following excitation wavelengths: Sytox Blue (405 nm), Calcein. AM (461 nm), Endosome-GFP (488 nm), LysoView 540 (540 nm), tissue reflectance (561 nm), and Cyanine-5 (633 nm). Detection channels were gated to appropriately isolate distinct fluorophore signals. Image Analysis. Sample viabilities were calculated using a custom MATLAB code (samples with <1000 total cells were excluded from further analysis). Additionally, the spatial information from the live cell stain (Calcein. AM) was isolated to create logical masks to compartmentalize C′ Dot signal within cells and ECM. After normalizing the C′ Dot signal to the brightest pixel in the channel, integrated and area-normalized C′ Dot signal was calculated for each compartment. Separately, vesicular structures (region diameter fixed at 1.26 μm) were identified in the cell compartment. Integrated C′ Dot intensity within vesicle boundaries was analyzed, as well as average number of vesicles per cell. Cells with no identifiable vesicles were excluded from this calculation due to poor alignment with the analyzed confocal slice (1 μm imaging depth).

Animals. All procedures were performed according to protocols approved by the Institutional Animal Care and Use Committee (IACUC) or Cornell University. Sprague Dawley male rats (n=22, 300-325 g; Charles River Laboratories, Wilmington, MA) were housed in pairs under a standard 12 h light/dark cycle starting at 6:00 a.m. The animals were allowed to move freely in their cages, fed a low-fluorescence commercial diet (Teklad 2914) and were allowed access to tap water.

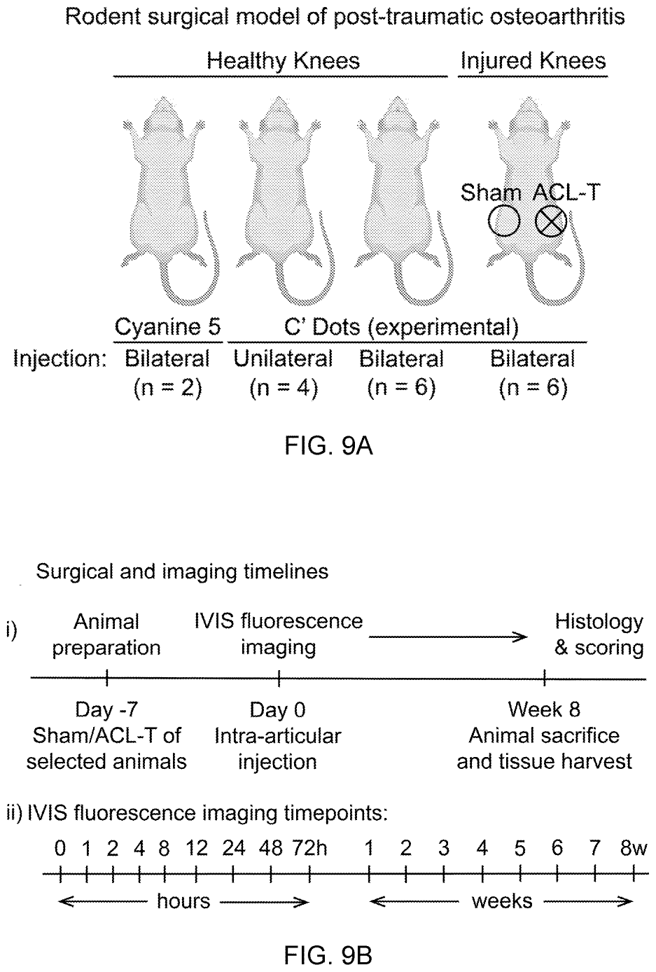

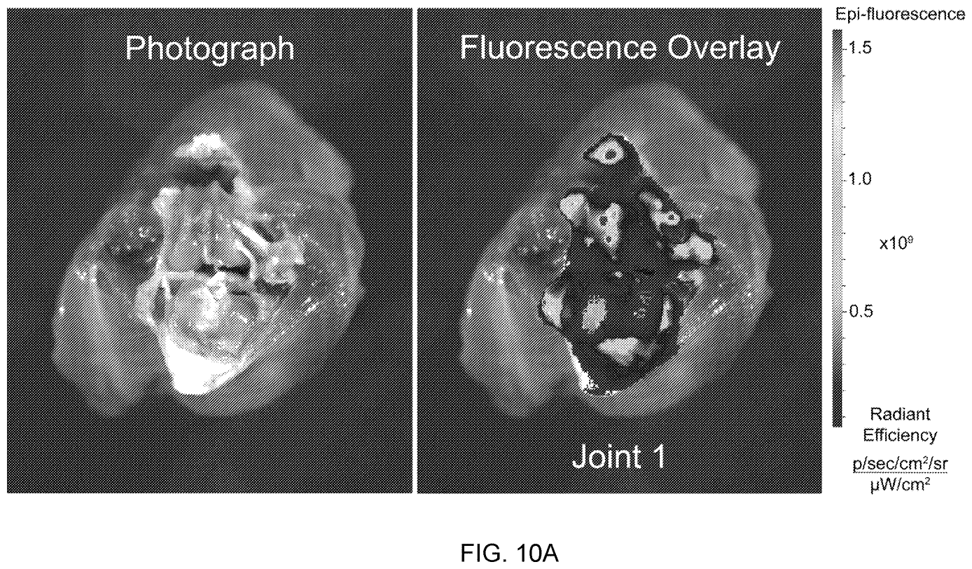

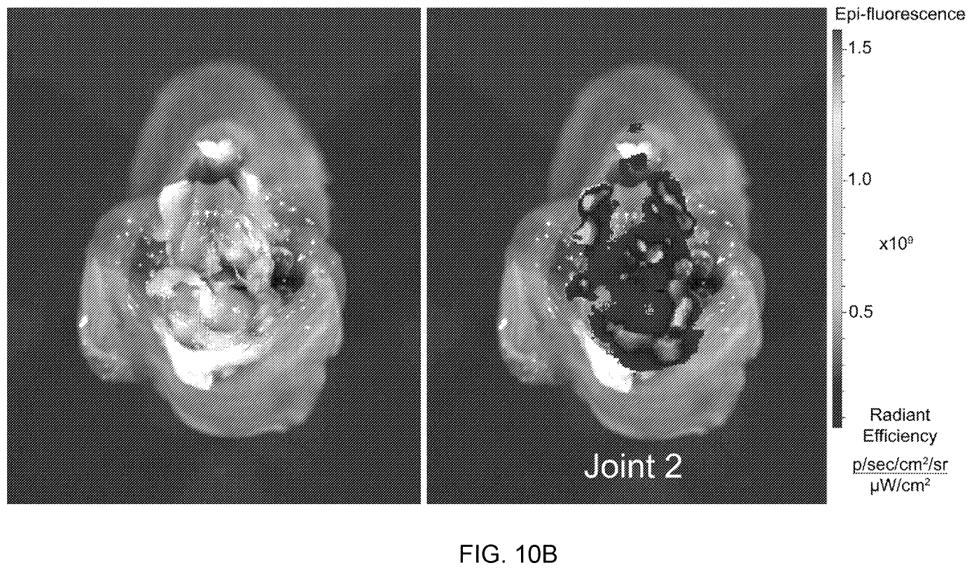

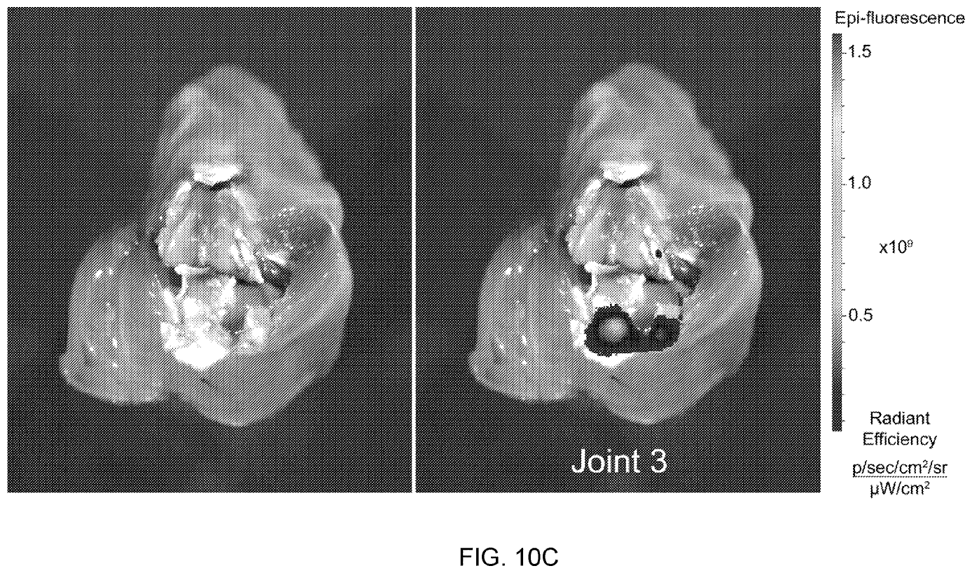

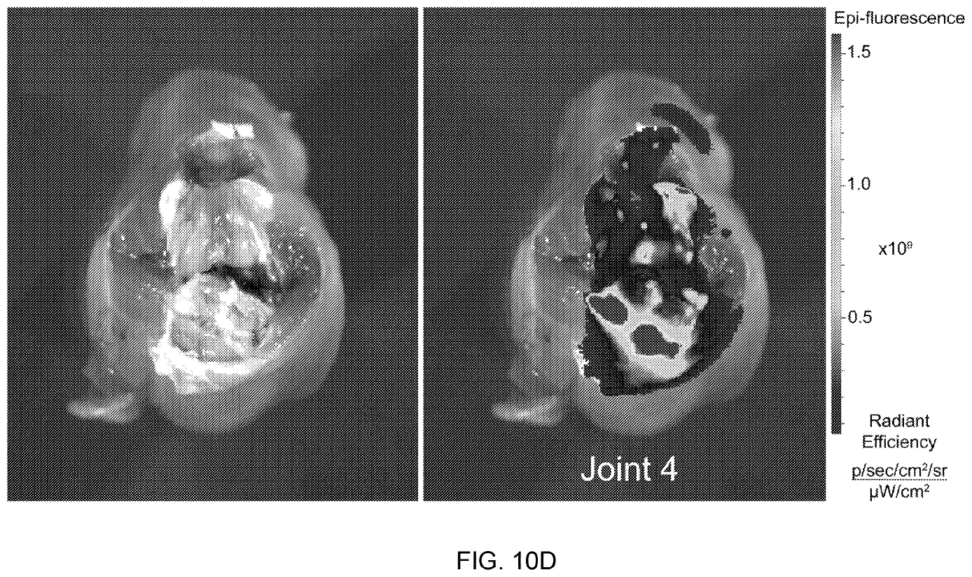

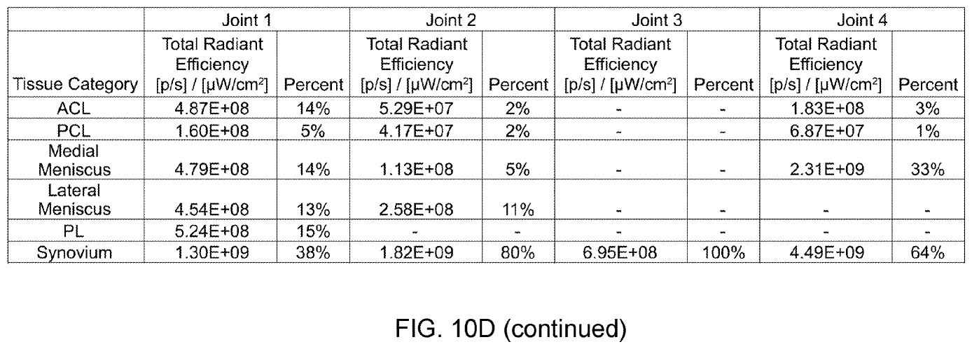

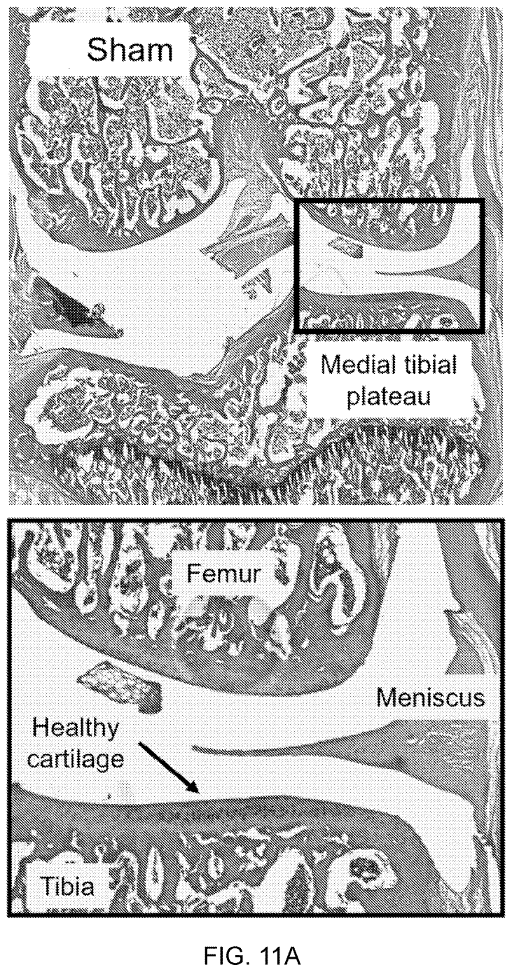

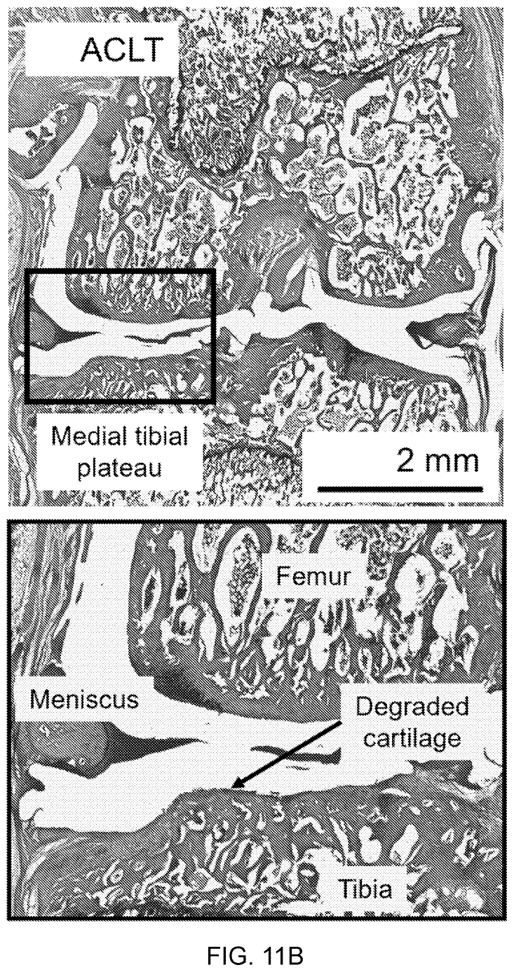

Animal Study Design. Rats were randomly assigned to (1) unoperated controls (naïve) and (2) those receiving ipsilateral anterior cruciate ligament transection (ACLT) and contralateral sham to model arthritic pathological joint changes (FIGS. 9A and B). Naïve rats were further split into pilot studies for intraarticular bilateral injection of Cy5 free fluorophore (n=2), unilateral C′ Dot injection with contralateral saline (n=4), and bilateral C′ Dot injection (n=4). Animals that received bilateral surgery were further split into groups where one group received no intraarticular injections (n=4) and one group received bilateral intraarticular injection of C′ Dots one week post-operatively (n=6). Selected animals underwent ACLT surgery on a randomly assigned knee via an open surgical approach under 2.5% isoflurane anesthesia at 1 L/min O₂, as previously described. Complete transection of the ACL was confirmed by a cranial drawer test. A single dose of Ethiqa XR (slow release buprenorphine) was administered subcutaneously at 0.65 mg/kg just prior to ACLT surgery. The animals could ambulate, eat, and drink ad libitum after surgery.

Intraarticular injection. Under 3% isoflurane anesthesia, the lower abdominal region (distal to sternum) including the front and back of both stifles was clipped to remove fur and sterilized with three alternating wipes of iodine and ethanol. Animals received a 25 μl parapatellar intraarticular injection of a sterile solution of 34 μM C′ Dots in saline, 10 mM Cy5 in saline, or saline via a 27 G x ½ needle with permanently attached syringe. Stifles were not flexed prior to the first IVIS image (0 h) to avoid leakage of the injected fluorescent material from the needle insertion site. Animals woke up to ambulate, distributing injectate throughout the joint, before being anesthetized again for the 1 h imaging timepoint.

In vivo longitudinal fluorescence imaging. Longitudinal fluorescence images of rats receiving Cy5 or C′ Dot intraarticular injections were recorded using the In Vivo Imaging System (IVIS) Kinetic (Perkin Elmer; Waltham, MA) over one week for Cy5 injection at the following timepoints: 0, 1, 2, 4, 8, 12, 24, 48, and 72 h, and at 7 d (FIG. 9S3). Rats receiving C′ Dot injections were imaged at the same initial timepoints (to 7 d) followed by imaging once per week for a total of 8 weeks. At each time point, rats were anesthetized with 3% isoflurane anesthesia at 1 L/min O₂, which was maintained through animal preparation and imaging. Each week, rat fur was clipped as described above and animal weights were recorded. IVIS imaging parameters were set manually for all rat images at all timepoints as follows: FOV: 22.4 cm, Exposure Time: 0.5 s, f Number: F/2, Bin size: 4, and subject height: 4 cm. Automated settings were also used to record a second image at each timepoint. Fluorescence was set at 640 nm/680 nm excitation/emission optimized for Cyanine-5 and C′ Dots.

IVIS image analysis. In Living Image software, ellipsoidal ROIs of identical dimensions (2.2×3.8 cm) oriented with a vertical long axis were manually aligned over the fluorescent signal captured in both stifles on background subtracted images. Total Radiant Efficiency (TRE) (area sum of ((p/sec/cm²/sr)/(ρW/cm²))) was recorded for each stifle at each time point for the duration of imaging. Average TRE from saline-injected stifles (n=4) was subtracted from all stifle TRE measurements as tissue fluorescence background. TRE was then normalized to the peak signal in each stifle for clearance analysis.

Clearance analysis. Normalized TRE for each stifle receiving Cy5 or C′ Dot intraarticular injection was fitted to exponential decay models from the time of peak signal to the last imaged timepoint. A single exponential model was fit to Cy5 decay (Eq 2) while a biexponential model was fit to C′ Dot decay in naïve and ACLT/sham stifles (Eq 3) using Origin Pro software.

$$y = A {e}^{- t / τ} + c \qquad ( 2 )$$

$$y = {A}_{1} {e}^{- t / {τ}_{1}} + {A}_{2} {e}^{- t / {τ}_{2}} + c \qquad ( 3 )$$

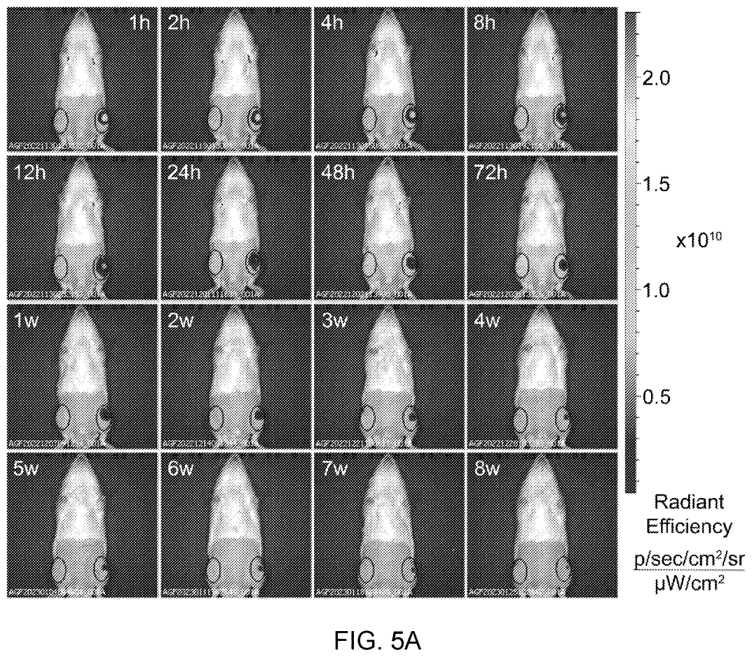

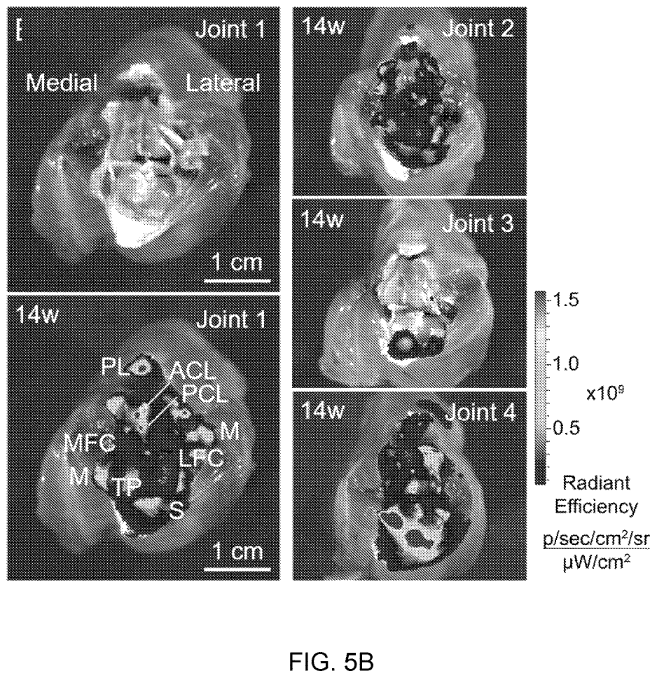

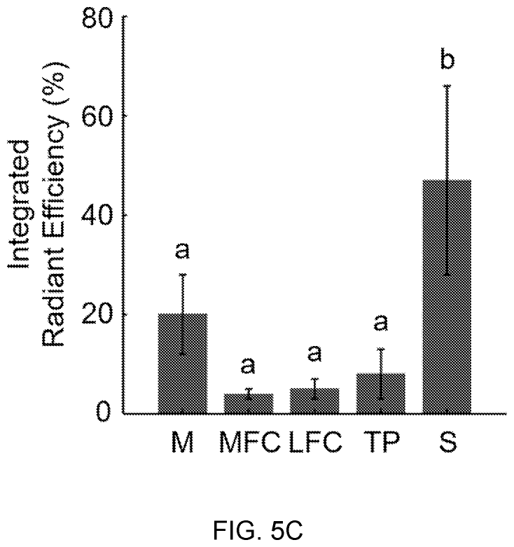

In these decay analyses, τ, τ₁, and τ₂ are time constants associated with A, A₁, and A₂, respectively. Half-lives (t_{1/2}) are calculated as t_{1/2}=ln(2)*τ. For proportions shown in FIG. 5B, the sum of all proportionality constants and c was used to normalize them. Proportionality constants tended to sum to 1.1-1.3.

Tissue processing and histology. Statistical analysis. Diffusion studies. A one-way ANOVA with Tukey's HSD (α=0.01) was performed on calculated D_{eff}. Chondrocyte studies. Viabilities, integrated signals, and area-normalized pixel intensities were compared using a linear mixed-effects model with Kenward-Roger approximation (α=0.05). Number of vesicles per cell were statistically compared using Students t-tests (α=0.05). Rodent study: Fitted time constants were statistically compared using linear mixed-effects model with Kenward-Roger approximation (α=0.05). Within each figure, shared letters denote no difference between groups.

Results. To assess C′ Dot penetration in cartilage, C′ Dots were incubated with cartilage explants in solution to allow passive diffusion for up to 2 hours (FIG. 2A). However, due to the finite observation time (i.e., confocal imaging on the scale of 5-10 minutes), particles diffused out of cartilage samples incubated for <0.5 h before penetration could be assessed (data not shown). This experimental artifact was reduced with incubation times >0.5 h. Therefore, one of the goals of this study was to determine the most appropriate time to diffuse for true estimations of C′ Dot diffusivity in cartilage. PEGylated C′ Dots penetrated the articular surface of patellofemoral groove cartilage explants within 0.5 h via passive diffusion and were present in deep zones of the cartilage within 2 h (FIG. 2B). C′ Dot fluorescence was visible in the cartilage matrix at all incubation time points, following a gradient from the articular surface to the center of the explant (FIG. 2B). Fluorescence profiles perpendicular to the articular surface were created from the C′ Dot fluorescence channel and D_{eff} was calculated in the range from 100-400 μm in depth from the articular surface using a custom MATLAB code (SI 2, FIG. 2C-D). Mean D_{eff} for C′ Dots were 7.9 μm²/s, 2.0 μm²/s and 1.6 μm²/s for 0.5 h, 1 h and 2 h, respectively (FIG. 2E). Short diffusion times (i.e. 0.5 h) produced inconsistent results, but calculated diffusivity was consistent between 1 h and 2 h observations (p=0.93). Thus, the mean D_{eff} at 2 h was regarded as the true diffusivity of C′ Dots in cartilage (FIG. 2E). 2 h incubations were used for all subsequent experiments.

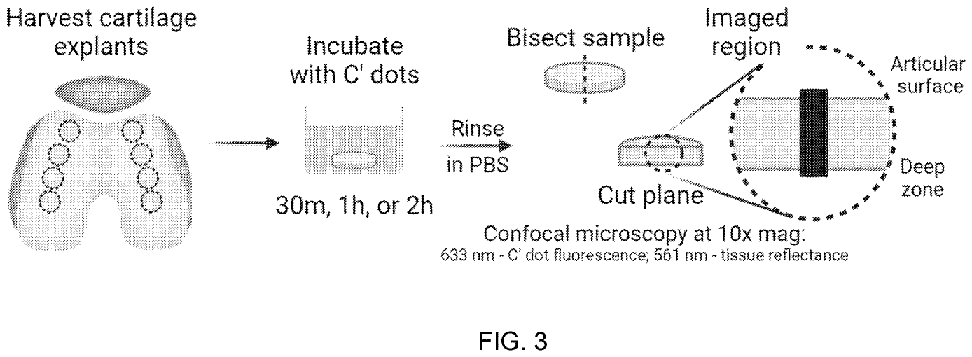

The data above demonstrate transport into cartilage, however, the fate of C′ Dots in intra-tissue and intra-cellular compartments was not clear. High magnification confocal imaging was performed to understand the distribution of C′ Dots between ECM and cells. Fluorescent C′ Dot puncta were observed within live cell cytoplasm after 24 hours of incubation in C′ Dot-media and 5 subsequent days of media incubation (FIG. 3A (left)). C′ Dot signal remained detectable intracellularly and in ECM from 0 to 5 days (FIG. 3A (right)). In separate endosome and lysosome co-localization experiments, it was observed that C Dots are partially co-localized with both fluorescent stains on this experimental time scale (FIG. 3B). C′ Dot signal was highly heterogeneous within cells: this organization into subcellular vesicular structures contributes to observed variations in cell pixel intensity (FIG. C (i)). Integrated C′ Dot signal in the ECM and cell compartment illustrates decreasing ECM signal over time, consistent with removal of C′ Dots during washing (FIG. 3C (ii)). While integrated C′ Dot signal in cells was less than integrated ECM signal due to low cellular density of articular cartilage (mean number of alive cells per explant: 0 d=750, 1 d=1100, 5 d=770) the concentration of C′ Dots within cell was greater (FIG. 3C (ii-iii)). C′ Dot concentration in the cells (e.g., area-normalized) (FIG. 3C (iii)) trended towards decreasing, with ~50% reduction observed from 0 d to 5 d (FIG. 3C (iii), p=0.79, p=0.97, respectively). Further analysis of the heterogeneous distribution of C′ Dot signal in cells revealed that integrated vesicular C′ Dot signal decreased from 1 d to 5 d (p=0.036) and was distinct from total cell and ECM compartments at 5 d (FIG. 3C (iv), p=0.037, p=0.014, respectively). Interestingly, the mean number of vesicles per cell ranged from 1 to 30 and did not change on this experimental timescale (FIG. 3C (iv) 0-1 d, p=0.95; 0-5 d, p=0.08; 1-5 d, p=0.23). Viabilities averaged 75% overall and were not different between C′ Dot and control groups at each timepoint (p>0.05).

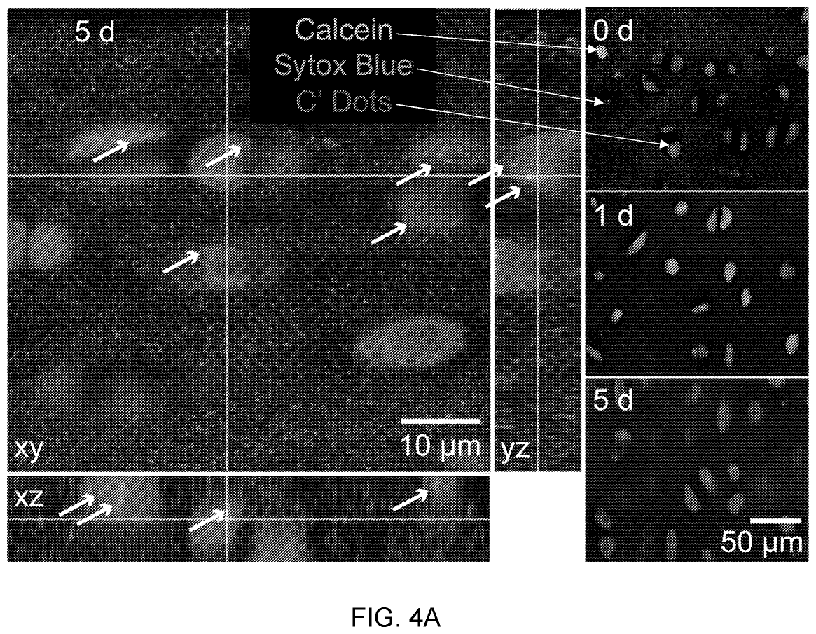

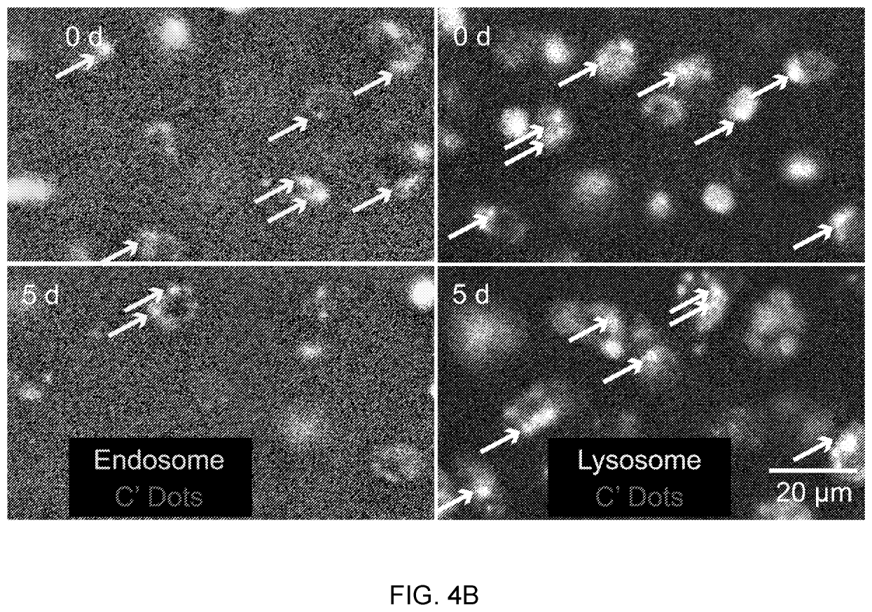

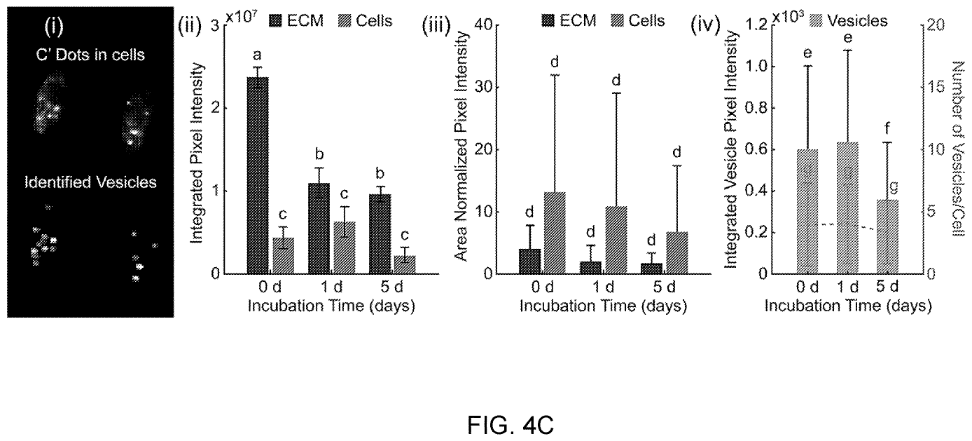

To assess in vivo joint clearance PK of C′ Dots under naïve and operated conditions, a rat model of knee arthritis (i.e., surgical ACLT) was utilized. Surgeries were performed in selected rats one week prior to injection and imaging. Rats received a single injection of C′ Dots and were imaged longitudinally over 8 weeks using the IVIS fluorescence system to observe clearance of C′ Dots from the whole joint (FIG. 4A). Separately, Cy5 dye was also injected into naïve rat knees and imaged for 7 days as a vehicle control. C′ Dot fluorescence was detected in knee joints immediately after injection (0 h, data not shown) and signal reached peak intensity within 2 hours in naïve rat knees and within 12 hours in operated knees. C′ Dot signal decreased over time and remained detectable up to 8 weeks post-injection (FIG. 4A). In contrast, Cy5 dye signal peaked at 0 h and fluorescence was undetectable after 3 days. C′ Dot fluorescence in unilaterally injected animals was not observed in the contralateral joint. Additionally, unilaterally and bilaterally injected animals only exhibited C′ Dot fluorescence at the injection site in the knee, with the exception of circumstantial observation of C′ Dot fluorescence above the bladder, consistent with renal clearance of C′ Dots observed in other systems. Since it was observed that C′ Dots were retained in the joint for 8 weeks, four pilot animals were sacrificed at 14 weeks post-injection for IVIS fluorescence imaging of the dissected joint tissues. Extended presence of C′ Dot fluorescence was observed in dissected naïve joints at this timepoint (FIG. 4B). C′ Dots were concentrated in the ligaments (PL, ACL, and PCL), menisci (M), and synovium (S), and present at lower levels in tibial plateau (TP) cartilage and condyles (MFC and LFC) (FIG. 4B). Given the untargeted surface chemistry of the C′ Dots, it was surprising that these particles were retained for longer than 3 months in naïve joints.

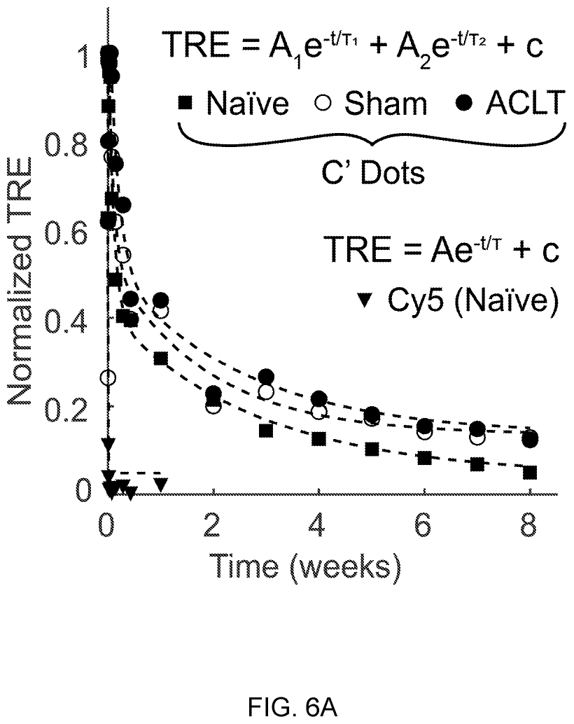

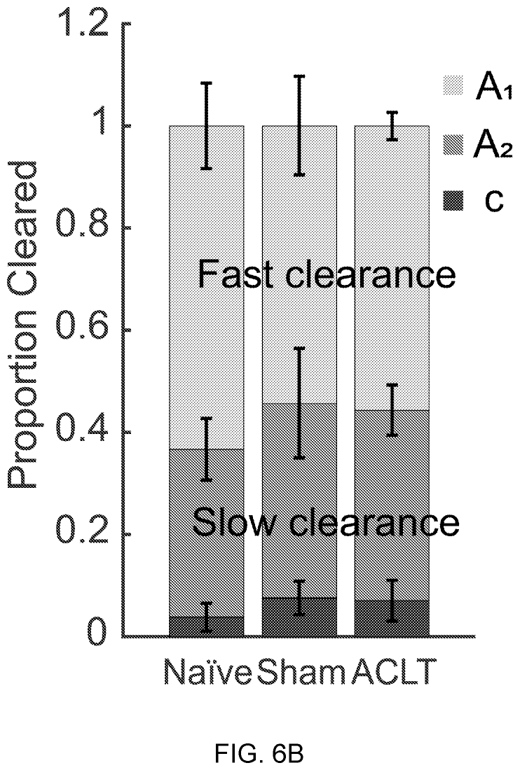

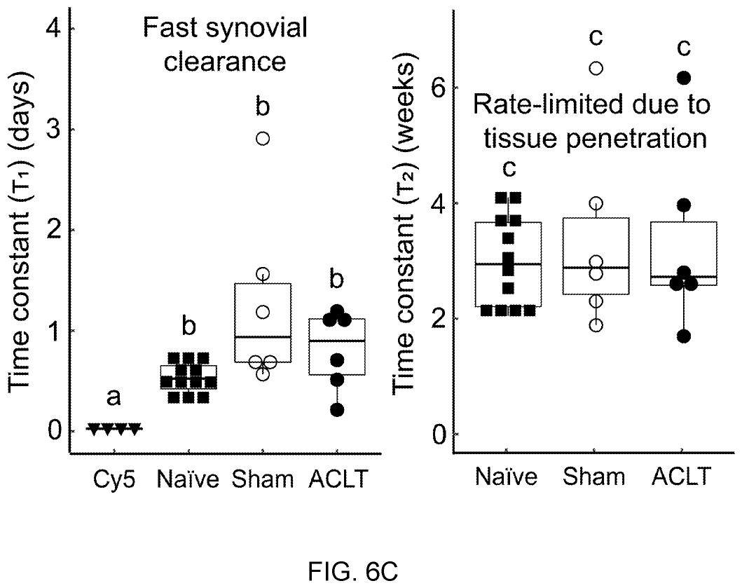

Cy5 signal became undetectable at 3 days and was recorded until 7 days post-injection. The clearance of Cy5 on this timescale was characterized as an exponential decay (Eq 2, τ=0.6 h, R²>0.98). Similarly, normalized C′ Dot fluorescence signal was fit to an exponential decay model (Eq 2). However, the model did not reflect the dataset since it underestimated at short times and overestimated at longer times (R²~0.9, RMSE~0.1). Thus, the clearance of C′ Dots was characterized as a biexponential decay (quick clearance: τ₁=18 h, slow clearance: τ₂=22 d; R²>0.95, RMSE~0.02) (FIG. 5A). These parameters are consistent with two time-distinct physiological processes, e.g. synovial clearance and tissue clearance. Normalized fitting parameters A₁, A₂, and c indicated that 64%/33% of injected C′ Dots in naïve stifles were described by shorter clearance and longer clearance, respectively, while 3% remains at 8 weeks. Similarly. 56%/37% of C′ Dots in all operated stifles were cleared in shorter/longer processes, respectively, while 7% remains at 8 weeks (FIG. 5B). τ₁ of C′ Dots was different between naïve and sham conditions (p=0.01) but not between naïve and ACLT (p=0.50) or ACLT and sham (p=0.28) (FIG. 5C). τ₂ of C′ Dots were not different between any condition: naïve and sham (p=0.84), naïve and ACL-T (p=0.79), or sham and ACL-T (p=0.99) (FIG. 6C).

Discussion the potential of C′ Dots for OA therapeutic delivery in the knee was investigated. An in vitro cartilage explant model was employed and characterized rapid penetration and diffusivity of C′ Dots in healthy tissue was characterized. It was discovered that C′ Dots are not internalized by chondrocytes on short timescales, in fact, chondrocytes require several hours to initiate internalization. This internalization dynamics was further investigated to discover that C′ Dots are retained intracellularly, primarily in vesicular structures, for longer than 5 days after removal from the media. In parallel in vivo studies, the joint clearance PK of a single dose of intraarticularly injected C′ Dots was investigated. A rat model of surgical arthritis was employed to characterize clearance from naïve and arthritic joints following intraarticular injection. It was observed that C′ Dot fluorescent signal remained detectable in intact joints for longer than 8 weeks post-injection and remained detectable in tissues of dissected joints at 14 weeks post-injection. Further, C′ Dot clearance from the joint is well represented by a biexponential clearance model, indicating two distinct time-dependent physiological processes consistent with synovial fluid clearance (fast process) and rate-limited tissue clearance (slow process). Overall, C′ Dots have great potential for enabling intra-cartilage delivery of conjugated OA therapeutics, utilizing a platform which has already shown safety in human clinical trials.

Ultrasmall C′ Dots penetrated the dense healthy cartilage matrix within the therapeutic synovial residence time of 2 hours corresponding to intraarticular injection. With a hydrodynamic diameter of 6 nm, matching the inter-GAG spacing and pore size of articular cartilage, the average calculated effective diffusion coefficients (~1.5-2.1 μm²/s) are consistent with literature for spherical solutes of this size.

C′ Dots were internalized within 24 hours by chondrocytes in 3D cartilage explants and remained detectable in compartmentalized ECM, cells, and vesicles over 5 days of incubation after removal of C′ Dots from the media. While it was expected that C′ Dots will be released from chondrocytes at later timepoints, these processing dynamics and unexpected retention kinetics observed on this timescale offers insight into chondrocyte dynamics for possible applications in drug delivery, as well as chondrocyte-mediated extension of joint residence times. In other studies, chondrocytes in 2D cultures robustly endocytose fluorescent particles as the primary (but not exclusive) uptake mechanism. Particles are internalized within 3-6 hours and retained for up to 48 hours. To our knowledge, this is the first quantification of compartmental retention of nanoparticles in chondrocytes for longer than 48 hours, and the first to use whole tissue explants to investigate chondrocyte processing dynamics in 3D. The role of chondrocytes in processing injected particles may offer a previously unexplored mechanism for increasing joint retention of OA therapeutics. Further investigation of not only bulk tissue transport kinetics, but complex cell-mediated sink/source modeling on the timescale of days to weeks is necessary to bridge the gap in timescale between in vitro studies (e.g., hours-days) and preclinical models (e.g. months-years).

In naïve and operated rat knees, C′ Dots exhibited slow clearance that was well described with two time constants (bi-exponential decay, R²>0.95), indicative of two time-dependent clearance mechanisms and consistent with synovial clearance and tissue clearance. This phenomenon is distinct from the clearance of Cy5 dye, which is modeled by a single time constant. Half-lives of C′ dots are more than an order of magnitude longer than Cy5 (t_{1/2,1}=13 h and 0.4 h, respectively), with the second half-life of C′ dots (t_{1/2·2}=15 days) characterizing greatly extended joint presence. Time constants were not different between unilateral and bilateral injection of C′ dots in naïve joints (p>0.05) or between unoperated and operated conditions (p>0.05). The shorter time constant describing C′ Dot clearance is comparable to half-lives of similarly sized solutes. The longer time constant, however, may be describing rate-limited release of C′ dots that initially diffused into tissues such as articular cartilage, meniscus, and ligaments, or were internalized by tissue-resident joint cells. In surgically operated stifles, the quicker half-life trended towards longer times compared to naïve stifles, although this effect only significant in Sham operated limbs. It is known that clearance of injected therapeutics from the joint is dependent on size, charge, hydrophobicity, and other factors, with disease state increasing joint residence time of nanoparticles due to synovial thickening. However, it was observed that disease state did not affect clearance PK, indicating that the C′ Dot vehicle is similarly qualified for extended joint retention in healthy and disease states. These observations inform the orthopedic community regarding nanoparticles of this size and design and suggest the potential of even untargeted C′ Dots for extended delivery of therapeutics in healthy joints and joints with mild arthritis consistent with this surgical model of PTOA.

### Example 2

This Example describes, inter alia, inorganic imaging agents, compositions, and methods of the present disclosure.

The effectiveness of intraarticular drug delivery approaches for osteoarthritis (OA) is hindered by the dense, avascular cartilage matrix and rapid synovial clearance time on the order of hours. Fluorescent organic-silica hybrid core-shell polyethylene-glycol coated (PEGylated) nanoparticles, C′ dots, have ultrasmall diameters (~6-7 nm) and versatile surface chemistry that facilitates conjugation of multiple different functional moieties. Ultrasmall C′ dots may overcome the dense cartilage matrix and rapidly diffuse through the GAG-governed pore size of ~6 nm. Surface functionalization of C′ dots with a cartilage-targeting peptide, such as Collagen-II binding peptide (Col-II), may be leveraged to tune effective diffusivity of C′ dots within cartilage. The aims were to i) characterize the penetration of C′ dots through the articular surface of healthy cartilage on a therapeutic timescale and ii) determine the effect of Col-II binding peptide functionalization on effective diffusivity in cartilage.

Methods. C′ dot Synthesis. C′ dots and Col-II binding peptide C′ dots were synthesized following previously established methods. Post-PEGylation, heterobifunctional PEGs were employed to introduce Col-IL binding functionalization via efficient click chemistry, with resulting experimental groups containing 2, 3, and 14 Col-II binding peptides per particle. Diffusion Studies. Cylindrical cartilage plugs (6 mm diameter and 1 mm deep, n=3-6 per condition) were cut from the patellofemoral groove of 1-3 day old bovine stifle joints and randomly assigned to C′ dot groups. C′ dots in solution passively diffused through the articular surface of submerged cartilage similarly to previously established methods. Imaging. After diffusion, samples were rinsed and bisected for confocal microscopy to assess C′ dot penetration. Cyanine-5 (Cy5) fluorescence of the C′ dots and cartilage reflectance were recorded utilizing the 633 nm and 561 nm laser lines, respectively. Image Analysis. The experiment was modeled as a 1D diffusion problem under the assumption that C′ dots only penetrated from the articular surface in the imaged region. Cy5 fluorescence from the cartilage matrix only was averaged in 6 m slices parallel to the articular surface and normalized using a custom MATLAB code. These data were fitted to a 1D diffusion equation derived from Fick's 2^{nd} law to obtain effective diffusivity, D_{ef} Statistical Analysis. A one-way ANOVA with Tukey's HSD (α=0.01) was performed on D_{eff} for non-functionalized C′ dots at all times and on non-functionalized and pooled functionalized C′ dots at 2 hours.

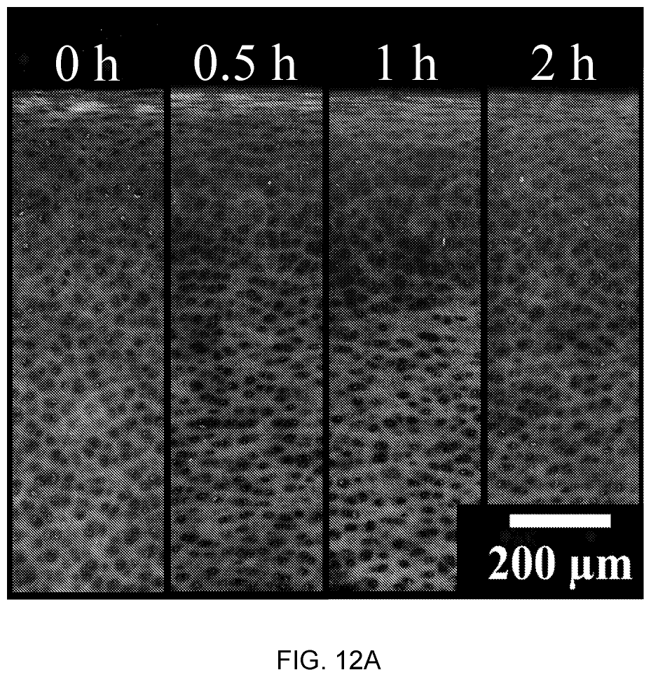

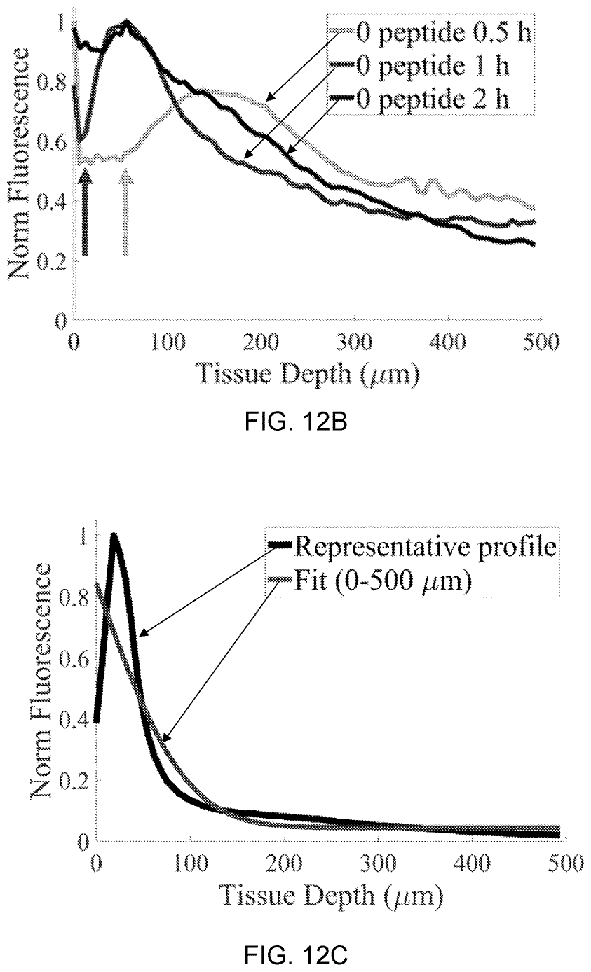

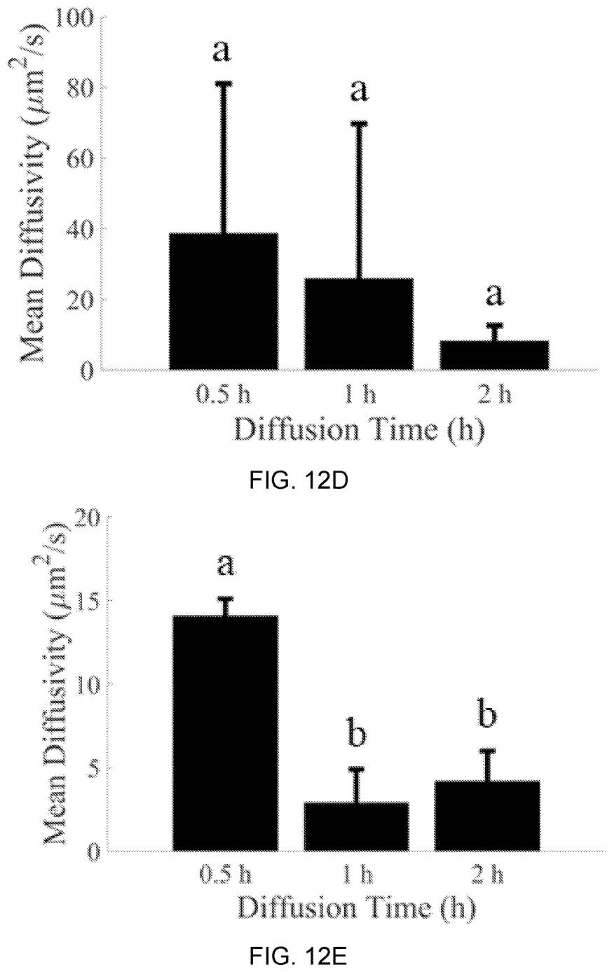

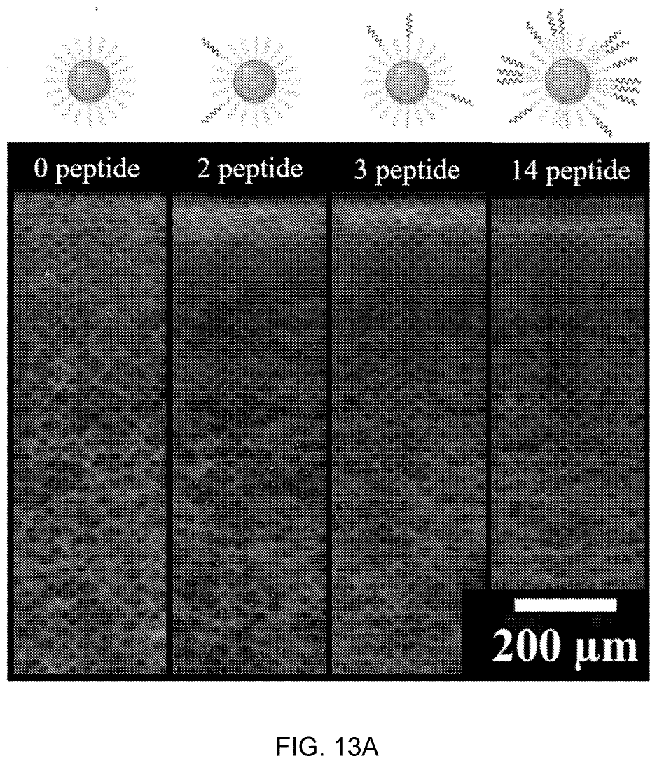

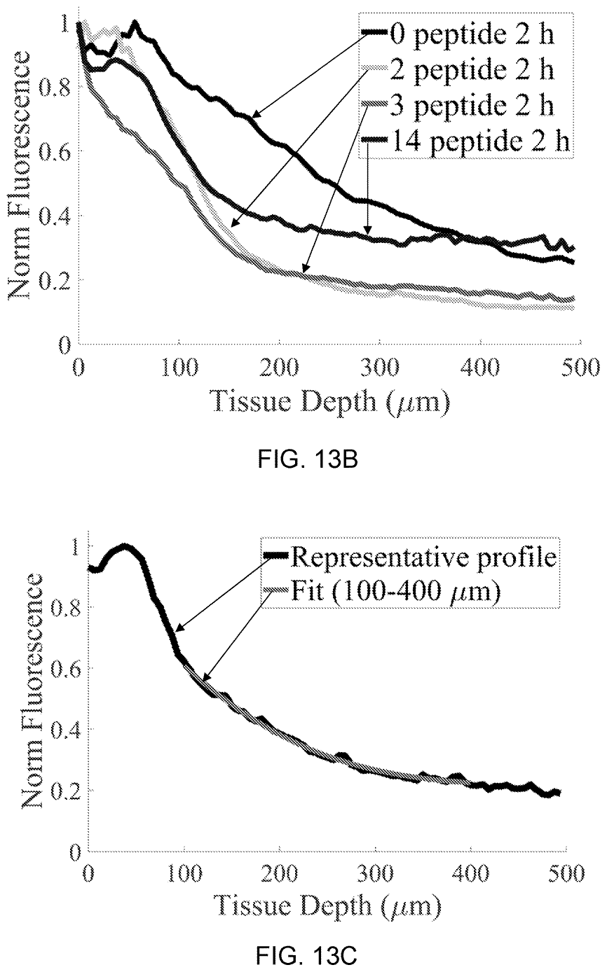

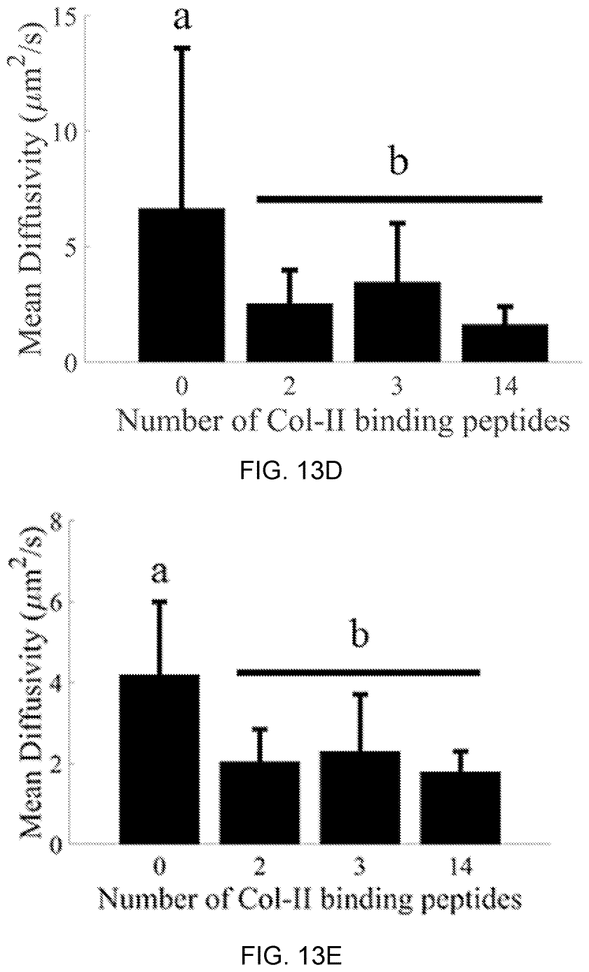

Results. Non-functionalized C′ dots penetrated the articular surface of patellofemoral groove cartilage explants within 0.5 h via passive diffusion and are present in deep zones of the cartilage within 2 h (FIG. 12A). Cy5 fluorescence was visible in the cartilage matrix at all imaged time points, following a gradient from the articular surface to the center of the explant (FIG. 12B). Average D_{eff} for non-functionalized C′ dots were calculated at 14.1 μm²/s, 2.9 μm²/s and 4.2 μm²/s for 0.5 h, 1 h and 2 h, respectively (FIG. 12C). Loss of C′ dots from the surface zone was noted at 0.5 h and 1 h, but this artifact was minimized after 2 h of diffusion (arrows, FIG. 12B). Functionalization of C′ dots with Col-II binding peptides decreased transport into cartilage (FIG. 13A&B), decreasing D_{eff} by ~50% (p<0.01, FIG. 13C). The number of Col-II binding peptides grafted to each particle did not affect D_{eff}, with <20% variation between groups (p>0.05, FIG. 13C).

Discussion Ultrasmall fluorescent C′ dots penetrated the dense healthy cartilage matrix within the therapeutic synovial residence time of 2 hours corresponding to intraarticular injection. With a hydrodynamic diameter of 6 nm, matching the inter-GAG spacing and pore size of articular cartilage, the average calculated effective diffusion coefficients (~1.8-4.2 μm²/s) are consistent with literature for spherical solutes of this size. Additionally, it is shown that Col-II binding peptide functionalization reduced the passive diffusion coefficient of the C′ dots, likely due to reversible binding interactions between the Col-II binding peptides and the cartilage matrix since the overall size of the functionalized C′ dots remained similar. Further characterization of the partition of C′ dots and Col-II binding C′ dots in cartilage and pharmacokinetics in vivo may elucidate additional tunable parameters due to peptide functionalization.

D_{eff} of C′ dots was reduced by half with the conjugation of Col-II binding peptide. Increased functionalization with Col-II or a chondrocyte-binding peptide may enable further tunability of C′ dot diffusivity in cartilage. On this experimental timescale, increased fluorescence intensity corresponding to resident chondrocytes was observed in some samples. Cellular localization of therapeutic payloads such as antibody fragments that block inflammatory cytokines may increase therapeutic potency, resulting in a chondroprotective effect and reduced matrix degradation. With a favorable safety profile previously shown in vivo and versatile functionalization capability, the C′ dot platform has potential to be a potent OA therapeutic for tissue targeting and delivery of therapeutic payloads for improved clinical outcomes.

C′ dots and Col-II binding peptide functionalized C′ dots penetrated healthy cartilage explants on a therapeutic timescale. Functionalization with Col-II binding peptide decreased effective diffusivity of C′ dots, consistent with binding to the matrix, which may serve as a tunable parameter for engineering a targeted therapeutic delivery vehicle for OA.

Intraarticular delivery of osteoarthritis therapeutics to chondrocytes is hindered by the dense cartilage matrix and short synovial half-life of small molecules on the order of hours. Residence time in the joint can be increased if the therapeutic molecule penetrates the articular surface of cartilage, effectively forming a drug depot. The metric of cartilage penetration, effective diffusivity, D_{eff}, accounts for the porosity and tortuosity of the cartilage matrix.

Nano delivery vehicles that can diffuse into cartilage have been investigated, but few have shown efficient clearance from the bloodstream and safety profiles in humans. Nano vehicles functionalized with Collagen-II binding peptides have increased joint half-life due to reversible ECM binding interactions. Cornell Prime Dots (C′ dots) are 5-6 nm poly(ethylene glycol) coated far-red fluorescent silica nanoparticles with engineerable surface modification using click chemistry. C′ dots exhibit favorable clearance and safety profiles in human clinical trials for oncology (e.g., NCT01266096), but this technology has not been applied to osteoarthritis.

Objective: Tune C′ dot penetration in healthy articular cartilage via functionalization with Collagen-II targeting peptides as a therapeutic delivery vehicle for osteoarthritis.

Methods. Experimental. Fluorescent Cyanine-5 (Cy5) encapsulating C′ dots were synthesized and functionalized with 2, 3, or 14 Collagen-II targeting peptides post-PEGylation using previously established methods. Cylindrical patellofemoral groove cartilage plugs of 6 mm dia.×1 mm height were obtained from 1-3 day old bovine stifle joints.

Image Analysis. In a custom MATLAB code, C′ dot fluorescence was isolated and segmented into 6 μm rectangles parallel to the articular surface. The average pixel intensity for each segment was used to construct a fluorescence profile (y) through the depth (x) of each sample. A 1D diffusion model derived from Fick's 2^{nd} Law was fit to this normalized profile, yielding effective diffusivities (D_{eff}) for C′ dots and targeted C′ dots in each cartilage sample.

$$\frac{y - {y}_{1}}{{y}_{0} - {y}_{1}} = erf ( \frac{x}{2 \sqrt{{D}_{eff} * t}} )$$

Statistical Analysis. A one-way ANOVA with Tukey's HSD (α=0.05) was performed. Shared letters denote no statistical difference between groups.

Results. C′ dots penetrate healthy articular cartilage progressively deeper over 2 h (FIG. 12A to E) Functionalization with Collagen-II targeting peptide reduces C′ dot effective diffusivity for enhanced tissue retention at 2 h (FIG. 13A to E).

Discussion & Conclusions. C′ dots penetrate the surface of articular cartilage within 2 h, i.e. within the expected half-life of C′ dots in the joint (FIGS. 12A and B) Loss of C′ dots from the surface cartilage is minimized at 2 h incubation where fit reliability is improved (FIGS. 12C and D).

At 2 h, effective diffusivity of C dots and targeted C′ dots is most precisely represented from 100-400 μm deep from the articular surface (FIG. 12C, FIG. 13C); the local D_{eff} of cartilage is reported to be consistent across this range.

Functionalization of Collagen-II targeting peptides on the C′ dot surface reduced D_{eff} in cartilage (FIG. 13D), consistent with reversible ECM binding interactions.

Targeted therapeutic delivery vehicles which penetrate cartilage on a timescale relevant to intraarticular injection may exhibit longer joint residence time for amelioration of cartilage deterioration.

Significance. Collagen-II targeting C′ dots penetrate articular cartilage for enhanced tissue retention and may serve as a therapeutic delivery vehicle for osteoarthritis.

### Example 3

This Example describes, inter alia, inorganic imaging agents, compositions, and methods of the present disclosure.

“Extended intra-articular presence of fluorescent C′ Dot nanoparticles in naïve and arthritic rat knee joints.”

C′ Dots clear by two distinct mechanisms in naïve and injured rat knees over 2 months. In naïve knees, C′ Dots remain localized intraarticularly to soft tissues and synovium for longer than 3 months after a single injection. As shown in FIG. 5C, C′ Dots clear slowly from naïve and injured rat knees and are retained in intraarticular tissues for 3+ months in dissected naïve knees after a single injection.

The data in FIG. 5C was obtained using the materials and methods described in EXAMPLE 1.

Although the present disclosure has been described with respect to one or more particular example(s), it will be understood that other examples of the present disclosure may be made without departing from the scope of the present disclosure.

1. A method of treating a subject diagnosed with or suspected of having or with increased risk due to injury of having rheumatoid arthritis and/or osteoarthritis comprising:
administering one or more inorganic nanotherapeutic agent(s) to the subject, wherein one or more symptoms of the individual's rheumatoid arthritis and/or osteoarthritis is treated.

2. The method of claim 1, wherein the inorganic nanotherapeutic agent(s) independently comprise one or more targeting group(s) and one or more therapeutic group(s), wherein the one or more targeting group(s) and the one or more therapeutic group(s) are independently disposed on a surface of the inorganic nanotherapeutic agent.

3. The method of claim 2, wherein the targeting group(s) is/are independently for each inorganic nanotherapeutic agent chosen from tissue targeting groups, tissue-resident cell targeting groups, and any combination thereof.

4. The method of claim 2, wherein the therapeutic group(s) is/are independently for each inorganic nanotherapeutic agent chosen from anti-inflammatory therapeutic groups, catabolic groups, anti-catabolic therapeutic groups, anabolic therapeutic groups, pro-anabolic groups, bioenergetic groups, viscosupplement groups, corticosteroid groups, steroid groups, antioxidant groups, gene therapy groups, nucleotide groups, and any combination thereof.

5. The method of claim 2, wherein the inorganic nanotherapeutic agent(s) independently further comprise one or more fluorescent group(s), one or more sulfur atom group(s), one or more radioisotope group(s), one or more heavy atom group(s), or any combination thereof, wherein the fluorescent group(s), the sulfur atom group(s), the radioisotope group(s), or the heavy atom group(s) are independently disposed on a surface of the inorganic nanoimaging agent.

6. The method of claim 1, wherein the inorganic nanotherapeutic agent(s) independently comprise(s) a silica matrix or an aluminosilicate matrix.

7. The method of claim 1, wherein inorganic nanotherapeutic agent(s) comprise a silica-poly(ethylene glycol)(PEG) core-shell particle.

8. The method of claim 1, wherein the inorganic nanotherapeutic agent(s) are independently chosen from nanoparticles, nanorings, and nanocages.

9. The method of claim 1, wherein at least a portion or all the inorganic nanoimaging agent(s) target(s) chondrocytes, tendon cells, ligament cells, meniscus cells, synovial cells, bone marrow cells, blood cells, lymphocytes, macrophages, fibroblasts, bone-lining cells, osteoblasts, osteoclasts, osteocytes, nerve cells, blood vessel cells, or any combination thereof, or a portion thereof.

10. The method of claim 1, wherein at least a portion of the inorganic nanotherapeutic agent(s) reside(s) in one or more joints(s) or any portion thereof, one or more musculoskeletal tissue(s) or any portion thereof, or any combination thereof in the subject for at least 14 days, at least 21 days, at least 28 days, or at least 3 months.

11. The method of claim 1, wherein the joint(s) are chosen from ankle joints, knee joints, hip joints, finger joints, wrists, elbows, temporomandibular, facet, and shoulder joints and/or the musculoskeletal tissue(s) are chosen from cartilage, synovial membrane, ligaments, tendons, tendon sheaths, bursas, bone, synovial fluid, meniscus, and meniscal enthesis, and any combination thereof.

12. The method of claim 1, wherein the musculoskeletal tissue(s) independently comprise cells and/or cell structures associated with cartilage, synovial membrane, ligaments, tendons, tendon sheaths, bursas, bone, synovial fluid, or meniscus, or any combination thereof and at least a portion of the inorganic nanotherapeutic agent(s) reside(s) in the cells and/or the cell structures.

13. The method of claim 1, wherein at least a portion of the inorganic nanotherapeutic agent(s) are taken up by in the chondrocytes, meniscus cells, ligament cells, tendon cells, lymphocytes, bone cells, or synovial cells, or any combination thereof of the individual.

14. The method of claim 1, wherein the inorganic nanotherapeutic agent(s) are present in composition and the composition is administered to the subject.

15. The method of claim 1, wherein the inorganic nanoimaging agent(s) are present in a composition at concentration of about 1 nanomolar to about 100 micromolar.

16. The method of claim 1, wherein the inorganic nanoimaging agent(s) is/are administered proximate to the joint(s) or any portion thereof, the musculoskeletal tissue(s), or any combination thereof of the subject.

17. The method of claim 1, wherein the administration is subcutaneous administration, intravenous administration, intraarticular administration, infrapatellar fat pad administration, or intramarrow administration.

18. The method of claim 1, wherein the administration comprises a single dose or multiple doses of inorganic nanotherapeutic agent(s).

19. The method of claim 1, wherein the administration is intraarticular administration and about 10 microliters to about 10 milliliters is administered.

20. The method of claim 1, wherein the inorganic nanotherapeutic agent(s) comprise a longest linear dimension of about 2 nanometers (nm) to about 1 micrometer and the administration is intraarticular.

21. The method of claim 1, wherein the inorganic nanoimaging agent(s) comprise a longest linear dimension of about 2 nanometers (nm) to about 10 nm and the administration is subcutaneous.

22. The method of claim 1, wherein the inorganic nanotherapeutic agent(s) do not comprise one or more fluorescent group(s), one or more sulfur atom group(s), one or more radioisotope group(s), one or more heavy atom group(s), or any combination thereof.

23. The method of claim 1, wherein the method does not comprise administration of any core-shell nanoparticles comprising one or more fluorescent group(s), one or more sulfur atom group(s), one or more radioisotope group(s), one or more heavy atom group(s), or any combination thereof.

24. The method of claim 1, wherein at least a portion of the inorganic therapeutic agents comprise one or more fluorescent group(s), one or more sulfur atom group(s), one or more radioisotope group(s), one or more heavy atom group(s), or any combination thereof, the method further comprises imaging one or more joints(s) or any portion thereof one or more musculoskeletal tissue(s) or any portion thereof, or any combination thereof the individual.

25. The method of claim 24, wherein the fluorescent group(s) is/are independently chosen from fluorescent organic dye group(s), fluorescent protein group(s), fluorescent peptide group(s), and any combination thereof.

26. The method of claim 24, wherein the musculoskeletal tissue(s) are chosen from cartilage, synovial membrane, ligaments, tendons, tendon sheaths, bursas, bone, synovial fluid, and meniscus, and any combination thereof.

27. The method of claim 24, wherein the musculoskeletal tissue(s) independently comprise cells and/or cell structures associated with cartilage, synovial membrane, ligaments, tendons, tendon sheaths, bursas, bone, synovial fluid, or meniscus, or any combination thereof.

28. The method of claim 24, wherein the imaging comprises:
directing excitation electromagnetic radiation into the sample or the subject, thereby exciting at least one of the one or more fluorescent group(s), if present, or one or more radioisotope(s), if present, or a combination thereof resulting in emission or excitation and emission of electromagnetic radiation from the fluorescent group(s), if present, or the radioisotope(s), if present, or the combination thereof;
detecting at least a portion of the emitted electromagnetic radiation, the detected electromagnetic radiation having been emitted by the fluorescent group(s), if present, or the radioisotope(s), if present, or the combination thereof; and
processing signals corresponding to the detected electromagnetic radiation to provide one or more image(s) of the joint(s) or the portion thereof, the musculoskeletal tissue(s) or the portion thereof, or the combination thereof in the sample or the subject.

29. The method of claim 28, wherein the electromagnetic radiation comprises one or more wavelength(s) from about 400 to about 1700 nm.

30. The method of claim 28, wherein the image(s) is/are fluorescence images.

31. The method of claim 28, wherein the electromagnetic radiation is directed into a region, wherein the region is within the sample or the subject and the region comprises the one or more joint(s) or the portion thereof, one or more musculoskeletal tissue(s) or the portion thereof, or the combination thereof.

32. The method of claim 24, wherein the imaging comprises multiphoton imaging.

33. The method of claim 28, wherein the imaging is super-resolution imaging.

34. The method of claim 24, wherein at least a portion or all the inorganic nanoimaging agent(s) is/are aluminosilicate nanoparticle(s), the aluminosilicate nanoparticle(s) independently comprising at least one organic dye molecule covalently bonded to the aluminosilicate network of the individual aluminosilicate nanoparticles and the imaging is optical super-resolution imaging.

35. The method of claim 34, wherein the image(s) resulting from the imaging are fluorescence image(s) or a sequence of fluorescence image(s) which can be processed to obtain an optical super-resolution image of the sample or portion thereof or the individual or a portion thereof.

36. The method of claim 24, wherein the imaging is an optical super-resolution microcopy method.

37. The method of claim 36, wherein the optical super-resolution microcopy method is ground state depletion (GSD) microscopy, stochastic optical reconstruction microscopy (STORM), direct stochastic optical reconstruction microscopy (dSTORM), stimulated emission and depletion (STED), or photoactivated localization microscopy (PALM).

38. The method of claim 24, further comprising an incubation time prior to the imaging.

39. The method of claim 1, further comprising an intravital fluorescence imaging method.

40. The method of claim 24, further comprising determining the treatment of the individual using the imaging.

41. The method of claim 24, further comprising diagnosing the individual using the imaging.

42. An inorganic nanotherapeutic agent comprising one or more targeting group(s) and one or more therapeutic group(s), wherein the one or more targeting group(s) and the one or more therapeutic group(s) are independently disposed on a surface of the inorganic nanotherapeutic agent.

43. The inorganic nanotherapeutic agent of claim 42, wherein the targeting group(s) is/are independently for each inorganic nanotherapeutic agent chosen from Type II collagen targeting peptides, and any combination thereof.

44. The inorganic nanotherapeutic agent of claim 42, wherein the therapeutic group(s) is/are independently for each inorganic nanotherapeutic agent chosen from IL-1 Ra (receptor agonist), TNF-α (alpha), or the like, or any combination thereof, and any combination thereof.

45. The inorganic nanotherapeutic agent of claim 42, wherein the inorganic nanotherapeutic agent(s) independently further comprise one or more fluorescent group(s), one or more sulfur atom group(s), one or more radioisotope group(s), one or more heavy atom group(s), or any combination thereof, wherein the fluorescent group(s), the sulfur atom group(s), the radioisotope group(s), the heavy atom group(s), or the targeting group(s), if present, are independently disposed on a surface of the inorganic nanoimaging agent.

46. The inorganic nanotherapeutic agent of claim 45, wherein the fluorescent group(s) is/are independently chosen from Cy5 groups, and any combination thereof.

47. The method of claim 45, wherein the radioisotope groups(s) is/are independently chosen from ¹²⁴I, ⁸⁹Zr, ¹¹C, ¹⁵O, ¹⁸F, ⁶⁴Cu, or ⁶⁸Ga, and any combination thereof.

48. The method of claim 45, wherein the heavy atom(s) is/are independently chosen from iodine atom, bromine atom, Au ions, Ag ions, Pb ions, Ti ions, Bi ions, Pt ions, In ions, Sn ions, Sb ions or Pd ions, and structural derivatives thereof, and any group derived therefrom, or any combination thereof

49. The inorganic nanoimaging agent of claim 42, wherein the inorganic nanotherapeutic agent comprises a silica matrix or an aluminosilicate matrix.

50. The inorganic nanoimaging agent of claim 42, wherein inorganic nanotherapeutic agent is a silica-poly(ethylene glycol)(PEG) core particle.

51. The method of claim 42, wherein the inorganic nanotherapeutic agent(s) are independently chosen from nanoparticles, nanorings, and nanocages.

52. The inorganic nanoimaging agent of claim 42, wherein inorganic nanotherapeutic agent is configured for use in a method of claim 1.

53. The inorganic nanoimaging agent of claim 42, wherein the inorganic nanotherapeutic agent comprises a longest linear dimension of about 2 nanometers (nm) to about 1 micrometer (micron).

54. The inorganic nanoimaging agent of claim 42, wherein the inorganic nanotherapeutic agent comprises a longest linear dimension of about 2 nm to about 10 nm.

55. A composition comprising one or more inorganic nanotherapeutic agent(s) of claim 42.

56. The composition of claim 55, wherein the composition comprises inorganic nanotherapeutic agent(s) at a concentration of 10 nanomolar to 100 micromolar.

57. The composition of claim 55, wherein the composition is suitable for subcutaneous administration, intravenous administration, intraarticular administration, infrapatellar fat pad administration, or intramarrow administration.

58. The composition of claim 57, wherein the composition is suitable for intraarticular administration.

59. The composition of claim 57, wherein the inorganic nanoimaging agent(s) comprise a longest linear dimension of about 2 nanometers (nm) to about 100 nanometers (nm) and the administration is intravenous.

60. The composition of claim 57, wherein the inorganic nanoimaging agent(s) comprise a longest linear dimension of about 2 nanometers (nm) to about 10 nm, and the administration is subcutaneous.


SEQUENCE LISTING (50 sequences)

SEQ ID NO: 1 - protein, 6 aa, synthetic construct
WYRGRL

SEQ ID NO: 2 - protein, 6 aa, synthetic construct
DPHFHL

SEQ ID NO: 3 - protein, 6 aa, synthetic construct
RVMLVR

SEQ ID NO: 4-10 - skipped (7 entries)

SEQ ID NO: 11 - protein, 12 aa, synthetic construct
GDYVIDWNFIEW

SEQ ID NO: 12 - protein, 12 aa, synthetic construct
TVGSFFVEWMMH

SEQ ID NO: 13 - protein, 12 aa, synthetic construct
DIGGWFVEWSLA

SEQ ID NO: 14 - protein, 12 aa, synthetic construct
DWGYFSWAYDSA

SEQ ID NO: 15 - protein, 12 aa, synthetic construct
DWYTVSWLTDSN

SEQ ID NO: 16 - protein, 12 aa, synthetic construct
DAYWHPVWVHDP

SEQ ID NO: 17 - protein, 12 aa, synthetic construct
DLQYWYPIWDTH

SEQ ID NO: 18 - protein, 12 aa, synthetic construct
HVYQKPSYWWYP

SEQ ID NO: 19 - protein, 12 aa, synthetic construct
TWHFVDFSADTH

SEQ ID NO: 20 - protein, 12 aa, synthetic construct
DYFTLDFTFDSW

SEQ ID NO: 21 - protein, 12 aa, synthetic construct
NQVYFHYFDLDF

SEQ ID NO: 22 - protein, 12 aa, synthetic construct
SPWWLWKAHNEA

SEQ ID NO: 23 - protein, 12 aa, synthetic construct
EVFNHYIQYSTE

SEQ ID NO: 24 - protein, 12 aa, synthetic construct
LPGMELFWNVAN

SEQ ID NO: 25 - protein, 12 aa, synthetic construct
DTFVFGSSKWRA

SEQ ID NO: 26 - protein, 12 aa, synthetic construct
SNNMRAPVNEIY

SEQ ID NO: 27 - protein, 20 aa, synthetic construct
RRAAAARRAAAARRAAAARR

SEQ ID NO: 28 - protein, 20 aa, synthetic construct
RRRRAARRRAARRRAARRRR

SEQ ID NO: 29 - protein, 32 aa, synthetic construct
ARRRAARAARRRAARAARRRAARAARRRAARA

SEQ ID NO: 30 - protein, 20 aa, synthetic construct
RRRRRRRRRRRRRRRRRRRR

SEQ ID NO: 31 - protein, 20 aa, synthetic construct
AKAKAKAKAKAKAKANANAN

SEQ ID NO: 32 - protein, 20 aa, synthetic construct
AKAKAKAKAKAKAKAFAFAF

SEQ ID NO: 33 - protein, 39 aa, synthetic construct
GSISIGIKCSPSIDLCEGQCRIRKYFTGYCSGDTCHCSG

SEQ ID NO: 34 - protein, 36 aa, synthetic construct
GSSCARPRENCNRMNILCCRGECVCPTFGDCFCYGD

SEQ ID NO: 35 - protein, 39 aa, synthetic construct
GSGVPINVKCRGSRDCLDPCKKAGMRFGKCINSKCHCTP

SEQ ID NO: 36 - protein, 39 aa, synthetic construct
GSGVPINVRCRGSRDCLDPCRRAGMRFGRCINSRCHCTP

SEQ ID NO: 37 - protein, 39 aa, synthetic construct
GSGVPINVRSRGSRDSLDPSRRAGMRFGRSINSRSHSTP

SEQ ID NO: 38 - protein, 39 aa, synthetic construct
GSVRIPVSCRHSGQCLRPCRDAGMRFGRCMNGRCDCTPR

SEQ ID NO: 39 - protein, 40 aa, synthetic construct
GSQVQTNVRCQGGSCASVCRREIGVAAGRCINGRCVCYRN

SEQ ID NO: 40 - protein, 12 aa, synthetic construct
DWRVIIPPRPSA

SEQ ID NO: 41 - protein, 12 aa, synthetic construct
RLDPTSYLRTFW

SEQ ID NO: 42 - protein, 12 aa, synthetic construct
HDSQLEALIKFM

SEQ ID NO: 43 - protein, 12 aa, synthetic construct
SFHQFARATLAS

SEQ ID NO: 44 - protein, 9 aa, synthetic construct
CKSTHDRLC

SEQ ID NO: 45 - protein, 9 aa, synthetic construct
CKPFDRALC

SEQ ID NO: 46 - protein, 21 aa, synthetic construct
KRKKKGKGLGKKRDPSLRKYK

SEQ ID NO: 47 - protein, 12 aa, synthetic construct
LRSRTKIIRIRH

SEQ ID NO: 48 - protein, 12 aa, synthetic construct
MPRRRRIRRRQK

SEQ ID NO: 49 - protein, 4 aa, synthetic construct
  SITE 2: note = D-arginine
YRFK

SEQ ID NO: 50 - protein, 4 aa, synthetic construct
  SITE 2: note = D-arginine
FRFK